(19) United States
(12) Patent Application Publication
(10) Pub. No.: US 2020/0246449 A1
(43) Pub. Date: Aug. 6, 2020

(54) INTRAVENOUS IMMUNOGLOBULIN COMPOSITIONS SPECIFIC FOR RESPIRATORY SYNCYTIAL VIRUS AND METHODS OF MAKING AND USING THE SAME

(71) Applicant: NanoBio Corporation, Ann Arbor, MI (US)

(72) Inventors: Ali I. Fattom, Ann Arbor, MI (US); Vira Bitko, Ann Arbor, MI (US); Shyamala GANESAN, Ann Arbor, MI (US)

(73) Assignee: NanoBio Corporation, Ann Arbor, MI (US)

(21) Appl. No.: 16/610,472

(22) PCT Filed: May 3, 2018
(86) PCT No.: PCT/US18/30920

§ 371 Date: Nov. 1, 2019

Related U.S. Application Data
(60) Provisional application No. 62/500,903, filed on May 3, 2017.

(51) Int. Cl. A61K 39/12; C07K 16/06; C07K 16/10; A61P 31/14

(52) U.S. Cl. CPC A61K 39/12; C07K 16/06; A61K 2039/543; A61P 31/14; C07K 16/1027

(57) ABSTRACT

The present disclosure relates to intravenous immunoglobulin (WIG) compositions specific for respiratory syncytial virus (RSV) and methods of making and using the same.

### CROSS-REFERENCE TO RELATED APPLICATIONS

This application is the U.S. National Stage of International Patent Application No. PCT/US2018/030920, filed May 3, 2018, which claims priority to US Provisional Application No. 62/500,903, filed on May 3, 2018, the contents of which are specifically incorporated herein by reference in their entirety.

### FIELD OF THE APPLICATION

The present application relates to the field of immunology, in particular, to compositions of polyclonal antibodies that can be administered to a subject to increase the subject's immunity to respiratory syncytial virus (RSV). The disclosed compositions are known as intravenous immunoglobulin (IVIG) products, and the present disclosure relates to making the disclosed IVIG compositions and using the compositions for treating or preventing RSV infections.

### BACKGROUND OF THE INVENTION

Respiratory Syncytial Virus (RSV) is a leading cause of serious respiratory disease in young children and the elderly worldwide and there is no vaccine available against this pathogen. RSV infects nearly all infants by age 2 and is the leading cause of bronchiolitis in children worldwide. It is estimated by the CDC that up to 125,000 pediatric hospitalizations in the United States each year are due to RSV, at an annual cost of over $300,000,000. Despite the generation of RSV-specific adaptive immune responses, RSV does not confer protective immunity and recurrent infections throughout life are common. While RSV is especially detrimental in very young infants whose airways are small and easily occluded, RSV is also widely becoming recognized as an important pathogen in transplant recipients, patients with chronic obstructive pulmonary disease (COPD), the elderly, as well as other patients with chronic lung disease, especially asthma. Recent data suggest that mortality for all ages combined has been approximately 30/100,000 from 1990-2000, with an annual average mortality of over 17,000 in the US. These numbers are likely grossly underestimated, as it has not been thoroughly examined in adults in a consistent manner. Thus, RSV not only causes significant exacerbated lung disease in young and old, but also is associated with a significant amount of mortality directly. Although anti-RSV antibodies are available and appear to alleviate severe disease, they perform only when given prophylactically and few other options exist for combating the RSV infections in susceptible patient populations.

In the late 1960s, attempts to vaccinate children with an alum-precipitated formalin-inactivated RSV vaccine preparation failed and caused severe exacerbated disease upon re-infection with live RSV. The clinical manifestations appeared to be a result of an enhanced Th2 disease, mucus production and eosinophilia that was not observed in non-vaccinated children. These same symptoms can occur in subsets of severely infected infants.

Extensive research into the development of viral vaccines to address RSV has met with limited success. Some of the major challenges for RSV vaccine development include early age of infection, evasion of innate immunity, failure of natural infection to induce immunity that prevents infection, and the demonstration of vaccine-enhance illness. Other options, such as monoclonal antibody treatments (e.g., SYNAGIS) have been attempted, but have proven to be costly and may not be effective across all serotypes of the virus.

Earlier disclosures describing nanoemulsion RSV vaccines include US 2010/0316673 for “Nanoemulsion Vaccines,” US 2013/0011443 for “Human Respiratory Syncytial Virus Vaccine,” and US 2013/0064867 for “Nanoemulsion Respiratory Syncytial Virus (RSV) Subunit Vaccine.” None of these disclosures teach or suggest IVIG compositions or the use thereof.

An example of a current drug used to prevent and treat RSV infection is SYNAGIS® (palivizumab). Palivizumab is a humanized monoclonal antibody (IgG) directed against an epitope in the A antigenic site of the F protein of RSV. In two Phase III clinical trials in the pediatric population, palivizumab reduced the risk of hospitalization due to RSV infection by 55% and 45%. Palivizumab is dosed once a month via intramuscular (IM) injection, to be administered throughout the duration of the RSV season. It is recommended for infants that are high-risk because of prematurity or other medical problems such as congenital heart disease.

Previous approaches to combating RSV have not only failed to treat the infection, some approaches have actually exacerbated it. For example, formalin inactivated vaccines have actually produced disease-enhancement. Moreover, the use of live attenuated vaccines has been met with limited success, as these vaccines were shown to be minimally immunogenic. Likewise, utilization of a recombinant F protein in vaccines has proven minimally effective, as it was discovered that the purified F protein is structurally immature and not appropriate for eliciting neutralizing antibodies.

Thus, there remains a need in the art for an effective therapeutic approach for treating and preventing RSV.

### SUMMARY OF THE INVENTION

The present invention provides a novel approach for inducing a protective immune response against RSV infection. In particular, the disclosure provides intravenous immunoglobulin (IVIG) compositions comprising one or more human antibodies that bind to RSV or an immunogenic fragment thereof. The plurality of antibodies is isolated from a donor that was immunized with a RSV vaccine, such as the disclosed nanoemulsion vaccine. The plurality of antibodies can be prepared from donor blood, serum, or plasma.

Thus, in one embodiment, the IVIG composition comprises polyclonal antibodies isolated from blood, serum or plasma from a donor that was immunized with a nanoemulsion RSV vaccine. The plurality of antibodies can comprise homologous or heterologous immunoglobulins.

In one aspect, prior to isolation of the plurality of antibodies from blood, serum, or plasma of the donor, the donor receives more than one dose of the nanoemulsion RSV vaccine. In some embodiments, the donor receives only one dose of the RSV vaccine.

In another aspect, immunization of the donor with a nanoemulsion RSV vaccine results in the generation of neutralizing antibody titers; for example, the neutralizing antibody titers present in the donor can range from about 2 to about 10⁶ IU/mL or more. For example, in some embodiment, immunization will result in about 1×10² neutralizing units/ml or more of antibodies in the donor.

In one aspect, the IVIG composition exhibits cross protection against an RSV strain not present in the nanoemulsion RSV vaccine, and in another aspect the IVIG composition exhibits multi-RSV epitope specificity.

In one aspect, the plurality of human antibodies comprises at least about 90% IgG and not more than about 10% non-IgG-contaminating proteins, or at least about 95% IgG, with not more than about 5% non-IgG-contaminating proteins, or at least about 96%, about 97%, about 98%, about 99%, or about 100% IgG.

In another aspect, the disclosure provides for methods of treating or preventing an RSV infection in a subject comprising administering to a subject an anti-RSV IVIG composition as described herein.

In some embodiments, the anti-RSV IVIG is administered to a subject to treat an active RSV infection, while in other embodiments, the anti-RSV IVIG is administered to a subject at risk of contracting RSV, such as a high risk subject, to prevent RSV infection. In some embodiments, the at-risk subject receiving the anti-RSV IVIG may be an infant, child, elderly, or immunocompromised. The anti-RSV IVIG can be administered to a subject via any pharmaceutically acceptable means. In some embodiments, the anti-RSV IVIG is administered intravenously.

In yet another aspect, the disclosure provides for methods of preparing a pharmaceutical composition comprising anti-RSV IVIG in a therapeutically effective dose for treatment or prevention of RSV infection. The method comprises (i) administering to a donor with a RSV vaccine; (ii) isolating from the donor a plurality of antibodies that bind to RSV or a RSV fragment; and (iii) formulating the plurality of antibodies into an IVIG composition.

In some embodiments, the RSV vaccine is a nanoemulsion vaccine, such as the disclosed nanoemulsion vaccine comprising whole virus, e.g., RSV-L19. In some embodiments, the RSV vaccine may be a subunit vaccine comprising, e.g., F protein, G protein, or a combination thereof.

In some embodiments, the RSV vaccine is administered to the donor in a dose corresponding to 1μg or more of F protein. For examples, the dose administered to the donor may correspond to 20, 50, or 100 or more μg or more of F protein. Similar doses can be calculated for G protein.

The nanoemulsion RSV vaccine used to generate antibodies in a donor comprises at least one nanoemulsion-inactivated RSV immunogen, and the immunogen can be whole virus, viral subunit(s) which are recombinant or isolated, or a combination thereof. The immune-enhancing nanoemulsion, or a dilution thereof, can comprise an aqueous phase, at least one oil, at least one surfactant, and at least one solvent. The nanoemulsion-inactivated RSV immunogen is adjuvanted by the nanoemulsion formulation to provide a non-infectious and immunogenic virus.

The RSV immunogen can be from any RSV strain, and can be whole virus or a subunit from an RSV virus. For example, the RSV immunogen can be any suitable RSV antigen, such as F protein, G protein, SH protein, nucleoprotein, phosphoprotein, matrix protein, large protein, or an immunogenic fragment of any of these proteins. The RSV virus subunit can be derived from any RSV strain, with an exemplary RSV strain being L19 (RSV-L19). In some embodiments, the nanoemulsion RSV vaccine comprises more than one strain of RSV. In some embodiments, the RSV virus in the vaccine may comprise at least one attenuating mutation.

In one aspect, the nanoemulsion RSV vaccine that is administered to a donor to generate an immune response in the donor comprises 1 82 g or greater of RSV F protein. For example, in some embodiments, the nanoemulsion RSV vaccine that is administered to a donor to generate an immune response in the donor comprises about 1, about 2, about 3, about 4, about 5, about 6, about 7, about 8, about 9, about 10, about 15, about 20, about 25, about 30, about 35, about 40, about 45, about 50, about 60, about 70, about 80, about 90, about 100, about 125, about 150, about 175, about 200, about 225, or about 250 μg or greater of RSV F protein.

In some embodiments, the nanoemulsion RSV vaccine used to elicit the immune response in the donor comprises an aqueous phase, at least one oil, at least one surfactant, and at least one solvent. In some embodiments, the nanoemulsion comprises an aqueous phase, about 1% to about 80% oil, about 0.1% organic solvent to about 50% organic solvent, and about 0.001% surfactant to about 10% surfactant, or a dilution of such a nanoemulsion. In some embodiments, the nanoemulsion comprises a cationic surfactant, chitosan, or glucan. In some embodiments, the aqueous phase of the nanoemulsion RSV vaccine is present in Phosphate Buffered Saline (PBS).

In some embodiments, the nanoemulsion comprises droplets having an average diameter of less than about 1000 nm, and in some embodiments, the nanoemulsion is not systemically toxic to the subject, produces minimal or no inflammation upon administration, or a combination of any of these features.

In some embodiments, the nanoemulsion RSV vaccine is administered to the donor via any pharmaceutically acceptable means, such as parenterally, orally or intranasally. Parenteral administration may be subcutaneous, intraperitoneal or intramuscular injection.

The foregoing general description and following brief description of the drawings and the detailed description are exemplary and explanatory and are intended to provide further explanation of the invention as claimed. Other objects, advantages, and novel features will be readily apparent to those skilled in the art from the following detailed description of the invention.

### DESCRIPTION OF THE FIGURES

FIG. 1 shows endpoint titer of RSV specific IgG in sera of BALB/c mice immunized with RSV. Only the group immunized with 20% W₈₀5EC nanoemulsion mixed with RSV F-protein responded to vaccination. The bar represents group average.

FIG. 2 shows endpoint titer of RSV specific IgG1 (FIG. 2A), IgG2a (FIG. 2B), IgG2b (FIG. 2C), and IgE (FIG. 2D) in sera of BALB/c mice immunized with nanoemulsion (NE)+RSV F protein. Sera were obtained two weeks after the second immunization.

FIG. 3 shows the results of vaccination of mice with nanoemulsion (NE)-F-protein, demonstrating that vaccination attenuates disease following intranasal challenge with live RSV. Immunized mice were vaccinated intranasally (i.n.) twice at day 0 and day 28 with NE+F-protein, F-protein alone or treated with PBS only. Control and vaccinated mice were challenged 2 weeks following the boost (i.n.) with 10⁵ PFU live RSV. The expression of virus transcripts were determined at day 8 post-infection via QPCR of lung RNA.

FIG. 4 shows that nanoemulsion (NE)-RSV immunization does not promote immunopotentiation when compared to non-vaccinated mice. Mice were vaccinated with NE-RSV as described below. Control and vaccinated mice were challenged at day 56. Airway hyperreactivity was assessed at day 8 post-challenge via plethysmography. Columns represent the increase in airway resistance following a single, optimized intravenous dose of methacholine.

FIG. 5 shows that inflammation and mucus production in nanoemulsion (NE)+F-protein vaccinated mice does not differ from controls. (FIG. 5A) depicts representative histology (Periodic Acid Schiff's, PAS; Hematoxylin and Eosin, H&E) from control RSV infected and NE+F-Protein vaccinated mice at day 8 post-infection. Eosinophils were not present. In (FIG. 5B), the expression of Muc5ac and Gob5 were assessed at day 8 post-infection via QPCR of lung RNA.

FIG. 6 shows that nanoemulsion (NE)+RSV F-protein vaccination promotes mixed Th1 and Th2 responses. Mice were vaccinated with NE+F-protein or F-protein alone, and challenged with live RSV. In (FIG. 6A), the expression of IL-12(p40) and (FIG. 6B) IL-17 cytokines were assessed from lung RNA via QPCR. In (FIG. 6C) Lung associated lymph node (LALN) cell suspensions were restimulated with RSV (MOI,0.5). Supernatants collected for analysis on the Bioplex to assay for cytokine production in each of the samples.

FIG. 7 shows IgG response in cotton rats following intramuscular immunization with nanoemulsion RSV vaccine at various doses. The results indicated that IgG response was optimized following two vaccinations at a dose corresponding to 4 μg of F protein.

FIG. 8 shows anti-F protein IgG levels in cotton rat sera at different timepoints following immunization with (1) IM NE01-RSV L19 vaccine (2) IM NE03-RSV L19 vaccine, (3) IM Alum FI-RSV vaccine, (4) RSV A2 infection, or (5) PBS.

FIG. 9 shows RSV A2 neutralization in cotton rat sera following immunization with (1) IM NE01-RSV L19 vaccine (2) IM NE03-RSV L19 vaccine, (3) IM Alum FI-RSV vaccine, (4) RSV A2 infection, or (5) PBS.

FIG. 10 shows viral clearance in the lung and nasal wash of cotton rats post-challenge with RSV after two immunizations with either (1) IM NE01-RSV L19 vaccine (2) IM NE03-RSV L19 vaccine, (3) IM Alum FI-RSV vaccine, (4) RSV A2 infection, or (5) PBS. IM injection with NE vaccine elicited robust protection against the RSV.

FIG. 11 shows lung histopathology results of cotton rats post-challenge with RSV after two immunizations with either (1) IM NE01-RSV L19 vaccine (2) IM NE03-RSV L19 vaccine, (3) IM Alum FI-RSV vaccine, (4) RSV A2 infection, or (5) PBS. IM injection with NE vaccine elicited robust protection against the RSV.

FIG. 12 shows that sera from NE-vaccinated animals is up to five times more neutralizing than existing RSV therapies (i.e. Synagis).

FIG. 13A shows the structure of RSV. Fusion Protein (F) and Attachment Protein (G) are present at the surface of the virion, while other components such as Matrix Protein (M), Nucleoprotein (N), RdRpol (L), Phosphoprotein (P), and the ribonucleoproteic complex are present in the interior of the virion. FIG. 13B shows the result of RSV antibodies induced via vaccination with an L19 nanoemulsion vaccine, with antibodies against N protein, P protein, M protein, G protein, F protein, and M2 protein detected. Reagents to measure NS1, NS2, SH and L antibodies were not available.

FIG. 14 shows the results of Western blots following 3 studies: (1) five (5) non-human primates immunized intranasally with 20% W₈₀5EC combined with 50 μg of F protein (L19 RSV virus based on F protein content) (FIG. 14A); (2) five (5) non-human primates immunized intramuscularly with 5% W₈₀5EC combined with 20 μg of F protein (L19 RSV virus based on F protein content) (FIG. 14B); and (3) four (4) different cotton rats immunized IM with 5% W₈₀5EC combined with 4, 8, or 15 μg of F protein (L19 RSV virus based on F protein content) (FIG. 14C). In both the NHP and CR studies, the animals were vaccinated three times at days 0, 28, and 56. Sera from each of the studies was pooled and evaluated for immunogenicity against various RSV proteins (FIGS. 14A, B, and C).

### DETAILED DESCRIPTION OF THE INVENTION

### I. Intravenous Immunoglobulin (IVIG) Compositions for Treating RSV

Immunoglobulin products from human plasma were first used in 1952 to treat immune deficiency. Intravenous immunoglobulin products (IVIG) comprise pooled immunoglobulin G (IgG) immunoglobulins. IVIGs are sterile and typically comprise more than 95% unmodified IgG, which has intact Fc-dependent effector functions and only trace amounts of immunoglobulin A (IgA) or immunoglobulin M (IgM). IVIG compositions have successfully been used to treat various immune conditions, including X-Linked Agammaglobulinemia (XLA) and Common Variable Immune Deficiency (CVID), but this type of immunotherapy has never been used to treat RSV.

Disclosed herein are compositions comprising IVIG specific for RSV. Typically, an IVIG-preparation suitable for treating a human is at least about 90% IgG, with not more than about 10% non-IgG-contaminating proteins. In some embodiments, an IVIG-preparation suitable for treating a human is at least about 95% IgG, with not more than about 5% non-IgG-contaminating proteins. In some embodiments, an IVIG-preparation suitable for treating a human is at least about 96%, about 97%, about 98%, about 99%, or about 100% IgG. Thus, disclosed herein are compositions comprising an anti-RSV IVIG, which comprises a plurality of antibodies that specifically bind to RSV. In some embodiments, the composition is a pharmaceutical composition comprising a plurality of antibodies that specifically bind to RSV and a pharmaceutically acceptable carrier.

Anti-RSV immunoglobulin preparations according to the present disclosure comprise a plurality of antibodies which can be prepared from any suitable starting materials. For example, immunoglobulin preparations can be prepared from donor blood, serum, or plasma, or the disclosed IVIG may comprise monoclonal or recombinant immunoglobulins. Thus, the plurality of antibodies are generally polyclonal, but in some embodiments the disclosed compositions may comprise monoclonal antibodies produced recombinantly based on the sequences of polyclonal antibodies isolated from a donor that was exposed to a nanoemulsion RSV vaccine. The plurality of antibodies preferably comprises human antibodies or humanized antibodies

In some embodiments, blood is collected from healthy donors after the donor has been exposed to RSV or an RSV vaccine (e.g., a nanoemulsion RSV vaccine as disclosed herein). Exposure to a nanoemulsion RSV vaccine results in the donor having high titers of antibody that specifically binds RSV. This type of IVIG may alternatively be referred to as a “hyperimmune globin” as it provides the recipient subject with “hyperimmunity,” or a larger-than-normal concentration of circulating antibodies. Administration of anti-RSV hyperimmune globulin provides “passive” immunity to the recipient subject, as the subject has an immediate increase in RSV-fighting antibodies. In contrast, vaccines provide “active” immunity, which takes much longer to achieve the purpose of fighting RSV because it requires the subject's immune system to produce anti-RSV antibodies in response to the vaccine. Thus, the disclosed anti-RSV IVIG is advantageous over traditional vaccination because it can be administered after a subject has become infected to provide immediate assistance to the subject's immune system.

In some embodiments, the donor may receive more than one dose of the RSV vaccine prior to isolation of the IVIG from the blood, serum, or plasma of the donor. For instance, the donor may receive 1, 2, 3, 4, 5, 6, 7, 8, 9, or 10 doses of a RSV vaccine, as disclosed herein, prior to isolation of the IVIG.

In some embodiments, immunization of the donor with the disclosed RSV vaccines results in the generation of robust neutralizing antibodies. For example, administration of one or two doses of a nanoemulsion RSV vaccine can result in neutralizing antibody titers ranging from about 2 to about 10⁶ IU/mL or more. For example, the serum concentration of anti-RSV antibodies in donors may be about 2, about 3, about 4, about 5, about 6, about 7, about 8, about 9, about 10, about 15, about 20, about 25, about 30, about 35, about 40, about 45, about 50, about 75, about 100, about 150, about 200, about 250, about 300, about 350, about 400, about 450, about 500, about 750, about 1000, about 2000, about 3000, about 4000, about 5000, about 7500, about 10000, about 50000, about 100000, about 1500000, or about 1000000 IU/mL. Thus, immunization with an RSV antibody as disclosed herein can produce at least about 1×10¹, at least about 5×10¹, at least about 1×10², at least about 5×10², at least about 1×10³, at least about 5×10³, at least about 1×10⁴, at least about 5×10⁴, at least about 1×10⁵, at least about 5×10⁵, or at least about 1×10⁶ neutralization units/ml of anti-RSV antibodies in the donor.

RSV vaccines formulated in nanoemulsion and administered intranasally (IN) or intramuscularly (IM) have been shown to elicit a robust immune response, including the production of antibodies that can be used in the preparation of an IVIG composition as disclosed herein. Moreover, nanoemulsion-inactivated and adjuvanted RSV vaccines are highly immunogenic in the universally accepted cotton rat model. Cotton rats elicited a rise in antibody titers after one immunization and a significant boost after the second immunization (approximately a 10-fold increase). The antibodies generated are highly effective in neutralizing live virus and there is a linear relationship between neutralization and antibody titers. Furthermore, antibodies generated in cotton rats showed cross protection when immunized with the RSV L19 strain and challenged with the RSV A2 strain. Both IM and IN immunization established memory that can be invoked or recalled after an exposure to antigen either as a second boost or exposure to live virus.

The nanoemulsion RSV vaccine can comprise whole virus (e.g., whole RSV-L19) or may comprise subunits of RSV (e.g., F and G proteins). Examples of nanoemulsion RSV vaccines that can be utilized in the methods of the invention include those described in US 2010/0316673 for “Nanoemulsion Vaccines,” US 2013/0011443 for “Human Respiratory Syncytial Virus Vaccine,” and US 2013/0064867 for “Nanoemulsion Respiratory Syncytial Virus (RSV) Subunit Vaccine,” the disclosures of which are specifically incorporated by reference.

Unlike prior antibody-based treatments for treating RSV (e.g., SYNAGIS®), the disclosed anti-RSV IVIG compositions are not monospecific. Whereas SYNAGIS® specifically binds only to the F protein on RSV, the disclosed anti-RSV IVIG has multi-epitope specificity, and therefore is expected to be efficacious against a wider range of RSV serotypes.

Pharmaceutical compositions suitable for use in the methods described below can comprise anti-RSV IVIG and a pharmaceutically acceptable carrier or diluent. The nanoemulsion RSV vaccines for use in producing the disclosed anti-RSV IVIG can be administered to a donor using any pharmaceutically acceptable method, such as for example, intranasal, buccal, sublingual, oral, rectal, ocular, parenteral (intravenously, intradermally, intramuscularly, subcutaneously, intracisternally, intraperitoneally), pulmonary, intravaginal, locally administered, topically administered, topically administered after scarification, mucosally administered, via an aerosol, or via a buccal or nasal spray formulation. Further, the nanoemulsion RSV vaccine can be formulated into any pharmaceutically acceptable dosage form, such as a liquid dispersion, gel, aerosol, pulmonary aerosol, nasal aerosol, ointment, cream, semi-solid dosage form, or a suspension. Further, the nanoemulsion RSV vaccine may be a controlled release formulation, sustained release formulation, immediate release formulation, or any combination thereof. Further, the nanoemulsion RSV vaccine may be a transdermal delivery system such as a patch or administered by a pressurized or pneumatic device (i.e., a “gene gun”).

Pharmacologically acceptable carriers for various dosage forms are known in the art. For example, excipients, lubricants, binders, and disintegrants for solid preparations are known; solvents, solubilizing agents, suspending agents, isotonicity agents, buffers, and soothing agents for liquid preparations are known. In some embodiments, the pharmaceutical compositions include one or more additional components, such as one or more preservatives, antioxidants, colorants, sweetening/flavoring agents, adsorbing agents, wetting agents and the like.

A. Immunoglobulin Preparation

Immunoglobulins can be isolated from blood, serum, or plasma by any suitable procedure known in the art, such as, for example, Cohn fractionation, ultracentrifugation, electrophoretic preparation, ion exchange chromatography, affinity chromatography, immunoaffinity chromatography, polyethylene glycol fractionation, or the like. See, e.g., Cohn et al., J. Am. Chem. Soc., 68:459-75 (1946); Oncley et al., J. Am. Chem. Soc. 71:541-50 (1949); Barundern et al., Vox Sang. 7:157-74 (1962); Koblet et al., Vox Sang. 13:93-102 (1967); U.S. Pat. Nos. 5,122,373 and 5,177,194; the disclosures of which are incorporated by reference herein.

In certain embodiments, immunoglobulin is prepared from gamma globulin-containing products produced by the alcohol fractionation and/or ion exchange and affinity chromatography methods well known to those skilled in the art. Purified Cohn Fraction II is commonly used. The starting Cohn Fraction II paste is typically about 95% IgG and is comprised of the four IgG subtypes. The different subtypes are present in Fraction II in approximately the same ratio as they are found in the pooled human plasma from which they are obtained. The Fraction II is further purified before formulation into an administrable product. For example, the Fraction II paste can be dissolved in a cold purified aqueous alcohol solution and impurities removed via precipitation and filtration. Following the final filtration, the immunoglobulin suspension can be dialyzed or diafiltered (e.g., using ultrafiltration membranes having a nominal molecular weight limit of less than or equal to 100,000 daltons) to remove the alcohol. The solution can be concentrated or diluted to obtain the desired protein concentration and can be further purified by techniques well known to those skilled in the art.

Preparative steps can be used to enrich a particular isotype or subtype of immunoglobulin. For example, sepharose chromatography can be used to enrich a mixture of immunoglobulins for IgG, or for specific IgG subtypes. See generally Harlow and Lane, Using Antibodies, Cold Spring Harbor Laboratory Press (1999); Harlow and Lane, Antibodies, A Laboratory Manual, Cold Spring Harbor Laboratory Press (1988); U.S. Pat. No. 5,180,810.

### II. Methods of Treating RSV With Anti-RSV IVIG

Disclosed herein are methods of treating or preventing an RSV infection in a subject comprising administering to the subject a composition comprising an anti-RSV IVIG. The subject administered the disclosed anti-RSV IVIG or pharmaceutical compositions may have already been infected with RSV or may be at risk of infection. While the disclosed methods of treatment are not specific for a single patient type, high-risk patients, including the elderly, children, babies, premature babies, and immunocompromised individuals, may be particularly desirable subjects. This is because the disclose anti-RSV IVIG creates immediate, “passive” immunity against RSV, as opposed to actively priming the immune system to fight the virus.

In general, IVIG antibodies circulate for about 1 month to fight infection, but the precise regimen used to treat or prevent RSV can differ according to the severity of the infection or risk of infection, as well as the age, size, sex, and general health of the subject receiving the disclosed anti-RSV IVIG. Thus, in some embodiments, the anti-RSV IVIG is administered once a day, once every two days, once every three days, once every four days, once every five days, once every six days, once a week, once every two weeks, once every three weeks, once a month, once every five weeks, once every six weeks, once every other month, once every three months, once every four months, once every five months, once every six months, once every seven months, once every eight months, once every nine months, once every ten months, once every eleven months, once a year or until the infection has been cured or the risk of infection has abated.

For the purposes of the disclosed methods, the dosage of the anti-RSV IVIG compositions is within a range of circulating concentrations that include the ED₅₀ with little or no toxicity. The dosage can vary within this range depending upon the dosage form employed, the severity of the illness, the age, sex, size, and general health of the subject, and the route of administration.

For example, in some embodiments, the anti-RSV IVIG is administered at a dose of about 1 mg/kg, about 5 mg/kg, about 10 mg/kg, about 20 mg/kg, about 30 mg/kg, about 40 mg/kg, about 50 mg/kg, about 100 mg/kg, about 150 mg/kg, about 200 mg/kg, about 250 mg/kg, about 300 mg/kg, about 350 mg/kg, about 400 mg/kg, about 450 mg/kg, about 500 mg/kg, about 550 mg/kg, about 600 mg/kg, about 650 mg/kg, about 700 mg/kg, about 750 mg/kg, about 800 mg/kg, about 850 mg/kg, about 900 mg/kg, about 950 mg/kg, or about 1000 mg/kg. In some embodiments, the anti-RSV IVIG is administered at a dose of about 50 mg, about 100 mg, about 500 mg, about 1000 mg, about 200 mg, about 3000 mg, about 3500 mg, about 4000 mg, about 4500 mg, about 5000 mg, about 5500 mg, about 6000, about 6500 mg, about 7000 mg, about 7500 mg, about 8000 mg, about 8500 mg, about 9000 mg, about 9500 mg, about 10000 mg, about 10500 mg, about 11000 mg, about 11500 mg, or about 12000 mg. In some embodiments, the anti-nicotine antibody is administered at a dose of 3000 mg, 3500 mg, 4000 mg, 4500 mg, 5000 mg, 5500 mg, 6000, 6500 mg, 7000 mg, 7500 mg, 8000 mg, 8500 mg, 9000 mg, 9500 mg, 10000 mg, 10500 mg, 11000 mg, 11500 mg, or 12000 mg.

### III. Methods of Producing IVIG for Treating RSV

Numerous suitable methods of isolating IVIG are known in the art (see, e.g., U.S. Pat. No. 7,138,120). Briefly, the starting material of the present purification process can be blood, serum, or plasma, but is advantageously an immunoglobulin-comprising crude plasma protein fraction. In general, IVIG are purified from normal human plasma or may originate from donors with high titers of specific antibodies, i.e., hyperimmune plasma.

In some embodiments, the anti-RSV IVIG comprises polyclonal antibodies that were isolated from the blood, serum, or plasma of a donor that was immunized or contacted with the disclosed nanoemulsion vaccines. In some embodiments, the anti-RSV IVIG comprises monoclonal antibodies, the sequences of which were derived from polyclonal antibodies that were isolated from the blood, serum, or plasma of a donor that was immunized or contacted with the disclosed nanoemulsion RSV vaccines. In such embodiments, a donor may be immunized with a RSV nanoemulsion vaccine as disclosed herein, and anti-RSV polyclonal antibodies are then isolated from the donor and screened for activity (i.e. binding affinity or viral neutralization).

The plurality of antibodies that make up anti-RSV IVIG can be isolated from a donor's blood, serum, or plasma at a suitable time after the donor has been immunized or contact with a nanoemulsion RSV vaccine. For example, anti-RSV antibodies for use as an IVIG composition may be isolated from a donor about 1, about 2, about 3, about 4, about 5, about 6, about 7, about 8, about 9, about 10, about 11, about 12, about 13, about 14, about 15, about 16, about 17, about 18, about 19, about 20, or about 21 or more days following immunization or contact with at least one of the disclosed nanoemulsion RSV vaccines.

A filter aid may or may not be used to purify the IVIG, depending on the isolation method used to obtain specific Cohn fractions (i.e. centrifugation or filtration).

A. RSV Immunogen for Nanoemulsion RSV Vaccine

RSV structure: RSV is an enveloped virus that contains a lipoprotein coat and a linear negative-sense RNA genome. The lipoprotein coat contains virally encoded F, G, and SH proteins. The F and G glycoproteins are the only two that target the cell membrane, and are highly conserved among RSV isolates.

Four of the viral genes code for intracellular proteins that are involved in genome transcription, replication, and particle budding, namely N (nucleoprotein), P (phosphoprotein), M (matrix protein), and L (“large” protein, containing the RNA polymerase catalytic motifs). The RSV genomic RNA forms a helical ribonucleoprotein (RNP) complex with the N protein, termed nucleocapsid, which is used as template for RNA synthesis by the viral polymerase complex.

The nanoemulsion RSV vaccine used to vaccinate a donor may comprise whole virus or viral subunits, such as a lipoprotein, nucleoprotein, phophoprotein, matrix protein, large protein, or an immunogenic fragment of any of these proteins, or a combination thereof. Thus, the immunogen utilized in the vaccine is not particularly limited. The simple mixing of a nanoemulsion with an RSV immunogen has been shown to produce both mucosal and system immune response. The mixing of the RSV immunogen with a nanoemulsion results in discrete immunogen particles in the oil core of the nanoemulsion droplet. The RSV immunogen is incorporated within the core and this allows it to be in a free form which promotes the normal antigen conformation.

The RSV vaccine can comprise whole RSV virus, including native, recombinant, and mutant strains of RSV, which is combined with the one or more RSV antigens. In one embodiment of the invention, the RSV virus can be resistant to one or more antiviral drugs, such as resistant to acyclovir. Any known RSV strain can be used in the vaccines of the invention. The nanoemulsion RSV vaccines can comprise RSV whole virus from more than one strain of RSV, as well as RSV antigens from more than one strain of RSV.

Examples of useful strains of RSV include, but are not limited to, any RSV strain, including subgroup A and B genotypes, as well as RSV strains deposited with the ATCC, such as: (1) Human RSV strain A2, deposited under ATCC No. VR-1540; (2) Human RSV strain Long, deposited under ATCC No. VR-26; (3) Bovine RSV strain A 51908, deposited under ATCC No. VR-794; (4) Human RSV strain 9320, deposited under ATCC No. VR-955; (5) Bovine RSV strain 375, deposited under ATCC No. VR-1339; (6) Human RSV strain B WV/14617/85, deposited under ATCC No. VR-1400; (7) Bovine RSV strain Iowa (FS1-1), deposited under ATCC No. VR-1485; (8) Caprine RSV strain GRSV, deposited under ATCC No. VR-1486; (9) Human RSV strain 18537, deposited under ATCC No. VR-1580; (10) Human RSV strain A2, deposited under ATCC No. VR-1540P; (11) Human RSV mutant strain A2 cpts-248, deposited under ATCC No. VR-2450; (12) Human RSV mutant strain A2 cpts-530/1009, deposited under ATCC No. VR-2451; (13) Human RSV mutant strain A2 cpts-530, deposited under ATCC No. VR-2452; (14) Human RSV mutant strain A2 cpts-248/955, deposited under ATCC No. VR-2453; (15) Human RSV mutant strain A2 cpts-248/404, deposited under ATCC No. VR-2454; (16) Human RSV mutant strain A2 cpts-530/1030, deposited under ATCC No. VR-2455; (17) RSV mutant strain subgroup B cp23 Clone 1A2, deposited under ATCC No. VR-2579; and (18) Human RSV mutant strain Subgroup B, Strain B1, cp52 Clone 2B5, deposited under ATCC No. VR-2542.

In another embodiment of the invention, the RSV vaccines of the disclosure are dosed according to their F protein content. Thus, the dose of the RSV vaccine to the donor may comprise about 1 μg or greater of RSV F protein. For example, the nanoemulsion RSV vaccine that is administered to a donor to generate an immune response in the donor can comprise about 1, about 2, about 3, about 4, about 5, about 6, about 7, about 8, about 9, about 10, about 15, about 20, about 25, about 30, about 35, about 40, about 45, about 50, about 60, about 70, about 80, about 90, about 100, about 125, about 150, about 175, about 200, about 225, or about 250 μg or greater of RSV F protein. The RSV immunogen is inactivated by the presence of the nanoemulsion adjuvant.

1. RSV Strain L19

In some embodiments, the RSV vaccine administered to a donor comprises whole virus of a particularly desirable strain or serotype of RSV including, for example, purified RSV strain L19 (RSV-L19). As disclosed in US 2013/0011443, RSV-L19 is a viral strain that is a hyperproducer of F and G viral proteins when compared to the commonly used RSV viral strain A2. In yet another embodiment, the RSV-L19 virus is attenuated RSV-L19. The more than 2-fold greater levels of the immunogenic F and G proteins found within RSV-L19 allows for the use of either attenuated or inactivated virus as a vaccine. RSV-L19 has been deposited with the American Type Culture Collection (ATCC).

In some embodiments, the disclosure provides for a method for preparing an immunogenic preparation, whereby RSV-L19 is genetically engineered with attenuating mutations and deletions resulting in an attenuating phenotype. The resulting attenuated virus is cultured in an appropriate cell line and harvested. The harvested virus is then purified free from cellular and serum components. The purified virus is then mixed in an acceptable pharmaceutical carrier for use as a vaccine composition. Thus, described are vaccine compositions comprising an RSV viral genome (such as RSV strain L19) comprising at least one attenuating mutation. In some embodiments, the vaccine compositions comprise an RSV viral genome (such as RSV strain L19) comprising nucleotide modifications denoting attenuating phenotypes.

The present disclosure provides methods, compositions and kits for the stimulation of an immune response to an RSV immunogen to isolate an anti-RSV IVIG from a donor whose immune response was stimulated. For example, cells infected with RSV L19 virus produce between 3-11 fold higher quantities of RSV viral proteins as compared to cells infected with RSV A2 virus (see Example 1).

RSV L19 strain was found to cause infection and enhanced respiratory disease (ERD) in mice. Moreover, published data has shown that RSV-L19 conferred protection without induction of ERD in mice when formulated with nanoemulsion. The RSV Strain L19 was isolated from an RSV-infected infant with respiratory illness in Ann Arbor, Mich. on 3 Jan. 1967 in WI-38 cells and passaged in SPAFAS primary chick kidney cells followed by passage in SPAFAS primary chick lung cells prior to transfer to MRC-5 cells (Herlocher 1999) and subsequently Hep2 cells (Lukacs et al. Am J Pathol. 169(3):977-86 (2006)). Comparison of RSV L19 genome (15,191-nt; GenBank accession number FJ614813) with the RSV strain A2 (15,222-nt; GenBank accession number M74568) shows that 98% of the genomes are identical. See Example 5. Most coding differences between L19 and A2 are in the F and G genes. Amino acid alignment of the two strains showed that F protein has 14 (97% identical) and G protein has 20 (93% identical) amino acid differences.

RSV L19 strain has been demonstrated in animal models to mimic human infection by stimulating mucus production and significant induction of IL-13 using an inoculum of 1×10⁵ plaque forming units (PFU)/mouse by intra-tracheal administration (Lukacs et al. Am J Pathol. 169(3):977-86 (2006)).

Importantly and uniquely, the RSV L19 viral strain is unique in that it produces significantly higher yields of F protein (approximately 10-30 fold more per PFU) than other RSV strains. F protein content may be a key factor in immunogenicity and the L19 strain currently elicits the most robust immune response. The L19 strain has a shorter propagation time and therefore can be more efficient from a manufacturing perspective. The results comparing RSV viral strains are provided in Table 9, Example 4.

In some embodiments, the RSV vaccines used for producing an anti-RSV IVIG can comprise RSV strain L19 and are cross-reactive against at least one other RSV strain (or cross-reactive against one or more RSV strains). Cross reactivity can be measured for example using ELISA method to see if the sera from vaccinated animals or individuals will produce antibodies against strains that were not used in the administered vaccine. Alternatively, immune cells will produce cytokines when stimulated in vitro using stains that were not used in the administered vaccine. Cross protection can be measured in vitro when antibodies in sera of animals vaccinated with one strain will neutralize infectivity of another virus not used in the administered vaccine.

For example, the RSV vaccines comprising RSV strain L19 can be cross reactive against one or more RSV strains selected from the group consisting of RSV strain A2 (wild type) (ATCC VR-1540P), RSV strain rA2cp248/404, RSV Strain 2-20, RSV strain 3-12, RSV strain 58-104, RSV strain Long (ATCC VR-26), RSV strain 9320 (ATCC VR-955), RSV strain B WV/14617/85 (ATCC VR-1400), RSV strain 18537 (ATCC VR-1580), RSV strain A2 cpts-248 (ATCC VR-2450), RSV strain A2 cpts-530/1009 (ATCC VR-2451), RSV strain A2 cpts-530 (ATCC VR-2452), RSV strain A2 cpts-248/955 (ATCC VR-2453), RSV strain A2 cpts-248/404 (ATCC VR-2454), RSV strain A2 cpts-530/1030 (ATCC VR-2455), RSV strain subgroup B cp23 Clone 1A2 (ATCC VR-2579), RSV strain Subgroup B, Strain B1, and cp52 Clone 2B5 (ATCC VR-2542).

2. RSV Subunit Immunogens

In some embodiments, the nanoemulsion RSV vaccine used to produce the disclosed IVIG product is a subunit vaccine. The RSV immunogen present in the nanoemulsion RSV vaccines of the invention can be any RSV immunogen as detailed above, such as an RSV surface antigen, such as a lipoprotein, nucleoprotein, phophoprotein, matrix protein, large protein, or an immunogenic fragment of any of these proteins. For example, F protein, G protein and antigenic fragments thereof can be obtained from any known RSV strain.

The RSV immunogen present in the vaccine of the invention can be (1) RSV F protein, (2) RSV G protein; (3) an immunogenic fragment of RSV F protein, (4) an immunogenic fragment of RSV G protein; (5) a derivative of RSV F protein; (6) a derivative of RSV G protein; (7) a fusion protein comprising RSV F protein or an immunogenic fragment of RSV F protein; (8) a fusion protein comprising RSV G protein or an immunogenic fragment of RSV G protein (9) or any combination thereof. Thus, in some embodiments, the RSV vaccine comprises at least one F protein immunogen and at least one G protein immunogen.

In an embodiment, an immunogenic fragment G protein of comprises at least 4 contiguous amino acids of the RSV G protein. In other embodiments, the RSV G protein fragment comprises about 4, about 5, about 10, about 15, about 20, about 25, about 50, about 75, about 100, about 125, about 150, about 175, about 200, about 225, about 250, about 275, about 280, about 285, about 289, about 290, about 295, or about 299 contiguous amino acids of RSV G protein. RSV G glycoprotein has about 289 to about 299 amino acids (depending on the virus strain). Conservative amino acid substitutions can be made in the G immunogenic protein fragments to generate G protein derivatives.

In another embodiment, an immunogenic fragment F protein of comprises at least 4 contiguous amino acids of the RSV F protein. In other embodiments, the RSV F protein fragment comprises about 4, about 5, about 10, about 15, about 20, about 25, about 50, about 75, about 100, about 125, about 150, about 175, about 200, about 225, about 250, about 275, about 300, about 325, about 350, about 375, about 400, about 425, about 450, about 475, or about 500 contiguous amino acids of RSV F protein. Conservative amino acid substitutions can be made in the F immunogenic protein fragments to generate F protein derivatives.

In some embodiments, the F protein derivatives are immunogenic and have a % identify to the F protein selected from the group consisting of 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, or 50%. In some embodiments, the G protein derivatives are immunogenic and have a % identify to the G protein selected from the group consisting of 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, or 50%.

In some embodiments, a vaccine composition may comprise isolated viral surface antigens, F and G proteins combined with isolated whole RSV virion particles, which are mixed together with a preferred oil-in-water nanoemulsion.

In one embodiment of the invention, the RSV vaccines comprise F protein and/or G protein of an RSV strain, such as but not limited to F protein or G protein of RSV strain L19. In another embodiment, the RSV vaccines comprise about 0.1 μp to about 100 μg, and any amount in-between, of RSV F protein or G protein, such as F protein or G protein of RSV strain L19. For example, the RSV vaccines can comprise about 0.1 μg, about 0.2 μg, about 0.3 μg, about 0.4 μg, about 0.5 μg, about 0.6 μg, about 0.7 μg, about 0.8 μg, about 0.9 μg, about 1.0 μg, about 1.1 μg, about 1.2 μg, about 1.3 μg, about 1.4 μg, about 1.5 μg, about 1.6 μg, about 1.7 μg, about 1.8 μg, about 1.9 μg, about 2.0 μg, about 2.1 μg, about 2.2 μg, about 2.3 μg, about 2.4 μg, about 2.5 μg, about 2.6 μg, about 2.7 μg, about 2.8 μg, about 2.9 μg, about 3.0 μg, about 3.1 μg, about 3.2 μg, about 3.3 μg, about 3.4 μg, about 3.5 μg, about 3.6 μg, about 3.7 μg, 3.8 μg, 3.9 μg, 4.0 μg, about 4.1 μg, 4.2 μg, 4.3 μg, 4.4 μg, 4.5 μg, 4.6 μg, about 4.7 μg, 4.8 μg, 4.9 μg, 5.0 μg, 5.1 μg, 5.2 μg, 5.3 μg, about 5.4 μg, 5.5 μg, 5.6 μg, 5.7 μg, 5.8 μg, 5.9 μg, about 6.0 μg, 6.1 μg, 6.2 μg, 6.3 μg, 6.4 μg, 6.5 μg, 6.6 μg, about 6.7 μg, 6.8 μg, 6.9 μg, 7.0 μg, 7.5 μg, 8.0 μg, about 8.5 μg, 9.0 μg, 9.5 μg, 10.0 μg, 10.5 μg, 11.0 μg, about 11.5 μg, 12.0 μg, 12.5 μg, 13.0 μg, 13.5 μg, 14.0 μg, about 14.5 μg, 15.0 μg, 15.5 μg, 16.0 μg, 16.5 μg, 17.0 μg, about 17.5 μg, 18.0 μg, 18.5 μg, 19.0 μg, 19.5 μg, 20.0 μg, about 21.0 μg, 22.0 μg, 23.0 μg, 24.0 μg, 25.0 μg, 26.0 μg, about 27.0 μg, 28.0 μg, 29.0 μg, 30.0 μg, 35.0 μg, 40.0 μg, about 45.0 μg, 50.0 μg, 55.0 μg, 60.0 μg, 65.0 μg, 70.0 μg, about 75.0 μg, 80.0 μg, 85.0 μg, 90.0 μg, 95.0 μg, 100 μg, about 125 μg, 150 μg, 175 μg, 200 μg, 225 μg, or about 250 μg of RSV F protein and/or G protein.

B. Nanoemulsions

Prior teachings related to nanoemulsions are described, for example, in U.S. Pat. Nos. 6,015,832; 6,506,803; 6,559,189; 6,635,676; 7,655,252; 7,767,216; 8,226,965; 8,232,320; 8,236,335; 8,703,164; 8,747,872; 8,771,731; 8,962,026; 9,131,680; and 9,259,407. In addition, prior teachings related to nanoemulsion vaccines are described in, for example, U.S. Pat. Nos. 7,314,624; 8,668,911; 8,877,208; 9,144,606; and 9,415,006. However, none of these references teach the methods, compositions and kits of the present invention.

The nanoemulsion compositions of the invention function as a vaccine adjuvant. Adjuvants serve to: (1) bring the RSV antigen—the substance that stimulates the specific protective immune response—into contact with the immune system and influence the type of immunity produced, as well as the quality of the immune response (magnitude or duration); (2) decrease the toxicity of certain antigens; (3) reduce the amount of RSV antigen needed for a protective response; (4) reduce the number of doses required for protection; (5) enhance immunity in poorly responding subsets of the population and/or (7) provide solubility to some vaccines components. The nanoemulsion vaccine adjuvants are particularly useful for adjuvanting RSV vaccines.

Nanoemulsions are oil-in-water emulsions composed of nanometer sized droplets with surfactant(s) at the oil-water interface. Because of their size, the nanoemulsion droplets are pinocytosed by dendritic cells triggering cell maturation and efficient antigen presentation to the immune system. When mixed with different antigens, nanoemulsion adjuvants elicit and up-modulate strong humoral and cellular T_{H}1-type responses as well as mucosal immunity.

In one embodiment, the nanoemulsion RSV vaccine comprises droplets having an average diameter of less than about 1000 nm and: (a) an aqueous phase; (b) about 1% oil to about 80% oil; (c) about 0.1% to about 50% organic solvent; (d) about 0.001% to about 10% of a surfactant or detergent; or (e) any combination thereof. In another embodiment of the invention, the nanoemulsion vaccine comprises: (a) an aqueous phase; (b) about 1% oil to about 80% oil; (c) about 0.1% to about 50% organic solvent; (d) about 0.001% to about 10% of a surfactant or detergent; and (e) at least one RSV immunogen. In another embodiment of the invention, the nanoemulsion lacks an organic solvent.

In one embodiment, the nanoemulsion vaccine droplets have an average diameter selected from the group consisting of less than about 1000 nm, less than about 950 nm, less than about 900 nm, less than about 850 nm, less than about 800 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, greater than about 50 nm, greater than about 70 nm, greater than about 125 nm, and any combination thereof.

In one embodiment, the nanoemulsion and/or nanoemulsion vaccine comprises a cationic surfactant, such as cetylpyridinium chloride (CPC). CPC may have a concentration in the nanoemulsion RSV vaccine of less than about 5.0% and greater than about 0.001%, or further, may have a concentration of less than about 5%, less than about 4.5%, less than about 4.0%, less than about 3.5%, less than about 3.0%, less than about 2.5%, less than about 2.0%, less than about 1.5%, less than about 1.0%, less than about 0.90%, less than about 0.80%, less than about 0.70%, less than about 0.60%, less than about 0.50%, less than about 0.40%, less than about 0.30%, less than about 0.20%, less than about 0.10%, greater than about 0.001%, greater than about 0.002%, greater than about 0.003%, greater than about 0.004%, greater than about 0.005%, greater than about 0.006%, greater than about 0.007%, greater than about 0.008%, greater than about 0.009%, and greater than about 0.010%.

In one embodiment, the nanoemulsion RSV vaccine comprises a non-ionic surfactant, such as a polysorbate surfactant, which may be polysorbate 80 or polysorbate 20, and may have a concentration of about 0.01% to about 5.0%, or about 0.1% to about 3% of polysorbate 80. The nanoemulsion RSV vaccine may further comprise at least one preservative. In some embodiments, the nanoemulsion RSV vaccine comprises a chelating agent.

In some embodiments, the nanoemulsion RSV vaccine further comprises an immune modulator, such as chitosan or glucan. An immune modulator can be present in the vaccine composition at any pharmaceutically acceptable amount including, but not limited to, from about 0.001% up to about 10%, and any amount in between, such as about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10%.

1. Stability of the Nanoemulsion RSV Vaccines of the Invention

The nanoemulsion RSV vaccines used for producing the disclosed IVIG compositions can be stable at about 40° C. and about 75% relative humidity for a time period of at least up to about 2 days, at least up to about 2 weeks, at least up to about 1 month, at least up to about 3 months, at least up to about 6 months, at least up to about 12 months, at least up to about 18 months, at least up to about 2 years, at least up to about 2.5 years, or at least up to about 3 years.

In some embodiments, the nanoemulsion RSV vaccines can be stable at about 25° C. and about 60% relative humidity for a time period of at least up least up to about 2 days, at least up to about 2 weeks, to about 1 month, at least up to about 3 months, at least up to about 6 months, at least up to about 12 months, at least up to about 18 months, at least up to about 2 years, at least up to about 2.5 years, or at least up to about 3 years, at least up to about 3.5 years, at least up to about 4 years, at least up to about 4.5 years, or at least up to about 5 years.

In some embodiments, the nanoemulsion RSV vaccines can be stable at about 4° C. for a time period of at least up to about 1 month, at least up to about 3 months, at least up to about 6 months, at least up to about 12 months, at least up to about 18 months, at least up to about 2 years, at least up to about 2.5 years, at least up to about 3 years, at least up to about 3.5 years, at least up to about 4 years, at least up to about 4.5 years, at least up to about 5 years, at least up to about 5.5 years, at least up to about 6 years, at least up to about 6.5 years, or at least up to about 7 years.

In some embodiments, the nanoemulsion RSV vaccines can be stable at about-20° C. for a time period of at least up to about 1 month, at least up to about 3 months, at least up to about 6 months, at least up to about 12 months, at least up to about 18 months, at least up to about 2 years, at least up to about 2.5 years, at least up to about 3 years, at least up to about 3.5 years, at least up to about 4 years, at least up to about 4.5 years, at least up to about 5 years, at least up to about 5.5 years, at least up to about 6 years, at least up to about 6.5 years, or at least up to about 7 years.

These stability parameters are applicable to nanoemulsion adjuvants and/or nanoemulsion RSV vaccines.

2. Immune Response Caused by RSV Vaccine

The immune response of the donor can be measured by determining the titer and/or presence of antibodies against the RSV immunogen after administration of the nanoemulsion RSV vaccine to evaluate the humoral response to the immunogen. Seroconversion refers to the development of specific antibodies to an immunogen and may be used to evaluate the presence of a protective immune response. Such antibody-based detection is often measured using Western blotting or enzyme-linked immunosorbent (ELISA) assays or hemagglutination inhibition assays (HAI). Persons of skill in the art would readily select and use appropriate detection methods.

Another method for determining the donor's immune response is to determine the cellular immune response, such as through immunogen-specific cell responses, such as cytotoxic T lymphocytes, or immunogen-specific lymphocyte proliferation assay. Additionally, challenge by the pathogen may be used to determine the immune response, either in the donor, or, more likely, in an animal model. A person of skill in the art would be well versed in the methods of determining the immune response of a subject and the invention is not limited to any particular method.

3. Components of the Nanoemulsion for RSV Vaccines

a. Droplet Size for RSV Vaccine Nanoemulsion

The nanoemulsion RSV vaccine can comprise droplets having an average diameter size of less than about 1,500 nm, less than about 1,450 nm, less than about 1,400 nm, less than about 1,350 nm, less than about 1,300 nm, less than about 1,250 nm, less than about 1,200 nm, less than about 1,150 nm, less than about 1,100 nm, less than about 1,050 nm, less than about 1,000 nm, less than about 950 nm, less than about 900 nm, less than about 850 nm, less than about 800 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, or any combination thereof. In one embodiment, the droplets have an average diameter size greater than about 125 nm and less than or equal to about 600 nm. In a different embodiment, the droplets have an average diameter size greater than about 50 nm or greater than about 70 nm, and less than or equal to about 125 nm.

b. Aqueous Phase for RSV Vaccine Nanoemulsion

The aqueous phase can comprise any type of aqueous phase including, but not limited to, water (e.g., H₂O, distilled water, purified water, water for injection, de-ionized water, tap water) and solutions (e.g., phosphate buffered saline (PBS) solution). In certain embodiments, the aqueous phase comprises water at a pH of about 4 to 10, preferably about 6 to 8. The water can be deionized (hereinafter “DiH₂O”). In some embodiments, the aqueous phase comprises phosphate buffered saline (PBS). The aqueous phase may further be sterile and pyrogen free.

c. Organic Solvents for RSV Vaccine Nanoemulsion

Organic solvents in the nanoemulsion RSV vaccines can include, but are not limited to, C₁-C₁₂ alcohol, diol, triol, dialkyl phosphate, tri-alkyl phosphate, such as tri-n-butyl phosphate, semi-synthetic derivatives thereof, and combinations thereof. In one aspect of the invention, the organic solvent is an alcohol chosen from a nonpolar solvent, a polar solvent, a protic solvent, or an aprotic solvent.

Suitable organic solvents for the nanoemulsion RSV vaccine include, but are not limited to, ethanol, methanol, isopropyl alcohol, glycerol, medium chain triglycerides, diethyl ether, ethyl acetate, acetone, dimethyl sulfoxide (DMSO), acetic acid, n-butanol, butylene glycol, perfumers alcohols, isopropanol, n-propanol, formic acid, propylene glycols, glycerol, sorbitol, industrial methylated spirit, triacetin, hexane, benzene, toluene, diethyl ether, chloroform, 1,4-dixoane, tetrahydrofuran, dichloromethane, acetone, acetonitrile, dimethylformamide, dimethyl sulfoxide, formic acid, semi-synthetic derivatives thereof, and any combination thereof.

d. Oil Phase for RSV Vaccine Nanoemulsion

The oil in the nanoemulsion RSV vaccines can be any cosmetically or pharmaceutically acceptable oil. The oil can be volatile or non-volatile, and may be chosen from animal oil, vegetable oil, natural oil, synthetic oil, hydrocarbon oils, silicone oils, semi-synthetic derivatives thereof, and combinations thereof.

Suitable oils include, but are not limited to, mineral oil, squalene oil, flavor oils, silicon oil, essential oils, water insoluble vitamins, Isopropyl stearate, Butyl stearate, Octyl palmitate, Cetyl palmitate, Tridecyl behenate, Diisopropyl adipate, Dioctyl sebacate, Menthyl anthranhilate, Cetyl octanoate, Octyl salicylate, Isopropyl myristate, neopentyl glycol dicarpate cetols, Ceraphyls®, Decyl oleate, diisopropyl adipate, C₁₂₋₁₅ alkyl lactates, Cetyl lactate, Lauryl lactate, Isostearyl neopentanoate, Myristyl lactate, Isocetyl stearoyl stearate, Octyldodecyl stearoyl stearate, Hydrocarbon oils, Isoparaffin, Fluid paraffins, Isododecane, Petrolatum, Argan oil, Canola oil, Chile oil, Coconut oil, corn oil, Cottonseed oil, Flaxseed oil, Grape seed oil, Mustard oil, Olive oil, Palm oil, Palm kernel oil, Peanut oil, Pine seed oil, Poppy seed oil, Pumpkin seed oil, Rice bran oil, Safflower oil, Tea oil, Truffle oil, Vegetable oil, Apricot (kernel) oil, Jojoba oil (simmondsia chinensis seed oil), Grapeseed oil, Macadamia oil, Wheat germ oil, Almond oil, Rapeseed oil, Gourd oil, Soybean oil, Sesame oil, Hazelnut oil, Maize oil, Sunflower oil, Hemp oil, Bois oil, Kuki nut oil, Avocado oil, Walnut oil, Fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, wintergreen leaf oil, flower oil, chamomile oil, clary sage oil, clove oil, geranium flower oil, hyssop flower oil, jasmine flower oil, lavender flower oil, manuka flower oil, Marhoram flower oil, orange flower oil, rose flower oil, ylang-ylang flower oil, Bark oil, cassia Bark oil, cinnamon bark oil, sassafras Bark oil, Wood oil, camphor wood oil, cedar wood oil, rosewood oil, sandalwood oil), rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, Oleic acid, Linoleic acid, Oleyl alcohol, Isostearyl alcohol, semi-synthetic derivatives thereof, and any combinations thereof.

The oil may further comprise a silicone component, such as a volatile silicone component, which can be the sole oil in the silicone component or can be combined with other silicone and non-silicone, volatile and non-volatile oils. Suitable silicone components include, but are not limited to, methylphenylpolysiloxane, simethicone, dimethicone, phenyltrimethicone (or an organomodified version thereof), alkylated derivatives of polymeric silicones, cetyl dimethicone, lauryl trimethicone, hydroxylated derivatives of polymeric silicones, such as dimethiconol, volatile silicone oils, cyclic and linear silicones, cyclomethicone, derivatives of cyclomethicone, hexamethylcyclotrisiloxane, octamethylcyclotetrasiloxane, decamethylcyclopentasiloxane, volatile linear dimethylpolysiloxanes, isohexadecane, isoeicosane, isotetracosane, polyisobutene, isooctane, isododecane, semi-synthetic derivatives thereof, and combinations thereof.

The volatile oil can be the organic solvent, or the volatile oil can be present in addition to an organic solvent. Suitable volatile oils include, but are not limited to, a terpene, monoterpene, sesquiterpene, carminative, azulene, menthol, camphor, thuj one, thymol, nerol, linalool, limonene, geraniol, perillyl alcohol, nerolidol, farnesol, ylangene, bisabolol, farnesene, ascaridole, chenopodium oil, citronellal, citral, citronellol, chamazulene, yarrow, guaiazulene, chamomile, semi-synthetic derivatives, or combinations thereof. In some embodiments, the volatile oil in the silicone component is different than the oil in the oil phase.

e. Surfactants for RSV Vaccine Nanoemulsion

The surfactant in the nanoemulsion RSV vaccine can be a pharmaceutically acceptable ionic surfactant, a pharmaceutically acceptable nonionic surfactant, a pharmaceutically acceptable cationic surfactant, a pharmaceutically acceptable anionic surfactant, or a pharmaceutically acceptable zwitterionic surfactant.

Exemplary useful surfactants are described in Applied Surfactants: Principles and Applications. Tharwat F. Tadros, Copyright 8 2005 WILEY-VCH Verlag GmbH & Co. KGaA, Weinheim ISBN: 3-527-30629-3), which is specifically incorporated by reference.

Further, the surfactant can be a pharmaceutically acceptable ionic polymeric surfactant, a pharmaceutically acceptable nonionic polymeric surfactant, a pharmaceutically acceptable cationic polymeric surfactant, a pharmaceutically acceptable anionic polymeric surfactant, or a pharmaceutically acceptable zwitterionic polymeric surfactant. Examples of polymeric surfactants include, but are not limited to, a graft copolymer of a poly(methyl methacrylate) backbone with multiple (at least one) polyethylene oxide (PEO) side chain, polyhydroxystearic acid, an alkoxylated alkyl phenol formaldehyde condensate, a polyalkylene glycol modified polyester with fatty acid hydrophobes, a polyester, semi-synthetic derivatives thereof, or combinations thereof.

Surface active agents or surfactants, are amphipathic molecules that consist of a non-polar hydrophobic portion, usually a straight or branched hydrocarbon or fluorocarbon chain containing 8-18 carbon atoms, attached to a polar or ionic hydrophilic portion. The hydrophilic portion can be nonionic, ionic or zwitterionic. The hydrocarbon chain interacts weakly with the water molecules in an aqueous environment, whereas the polar or ionic head group interacts strongly with water molecules via dipole or ion-dipole interactions. Based on the nature of the hydrophilic group, surfactants are classified into anionic, cationic, zwitterionic, nonionic and polymeric surfactants.

Suitable surfactants include, but are not limited to, ethoxylated nonylphenol comprising 9 to 10 units of ethyleneglycol, ethoxylated undecanol comprising 8 units of ethyleneglycol, polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan monolaurate, sorbitan monopalmitate, sorbitan monostearate, sorbitan monooleate, ethoxylated hydrogenated ricin oils, sodium laurylsulfate, a diblock copolymer of ethyleneoxyde and propyleneoxyde, Ethylene Oxide-Propylene Oxide Block Copolymers, and tetra-functional block copolymers based on ethylene oxide and propylene oxide, Glyceryl monoesters, Glyceryl caprate, Glyceryl caprylate, Glyceryl cocate, Glyceryl erucate, Glyceryl hydroxysterate, Glyceryl isostearate, Glyceryl lanolate, Glyceryl laurate, Glyceryl linolate, Glyceryl myristate, Glyceryl oleate, Glyceryl PABA, Glyceryl palmitate, Glyceryl ricinoleate, Glyceryl stearate, Glyceryl thiglycolate, Glyceryl dilaurate, Glyceryl dioleate, Glyceryl dimyristate, Glyceryl disterate, Glyceryl sesuioleate, Glyceryl stearate lactate, Polyoxyethylene cetyl/stearyl ether, Polyoxyethylene cholesterol ether, Polyoxyethylene laurate or dilaurate, Polyoxyethylene stearate or distearate, polyoxyethylene fatty ethers, Polyoxyethylene lauryl ether, Polyoxyethylene stearyl ether, polyoxyethylene myristyl ether, a steroid, Cholesterol, Betasitosterol, Bisabolol, fatty acid esters of alcohols, isopropyl myristate, Aliphati-isopropyl n-butyrate, Isopropyl n-hexanoate, Isopropyl n-decanoate, Isoproppyl palmitate, Octyldodecyl myristate, alkoxylated alcohols, alkoxylated acids, alkoxylated amides, alkoxylated sugar derivatives, alkoxylated derivatives of natural oils and waxes, polyoxyethylene polyoxypropylene block copolymers, nonoxynol-14, PEG-8 laurate, PEG-6 Cocoamide, PEG-20 methylglucose sesquistearate, PEG40 lanolin, PEG-40 castor oil, PEG-40 hydrogenated castor oil, polyoxyethylene fatty ethers, glyceryl diesters, polyoxyethylene stearyl ether, polyoxyethylene myristyl ether, and polyoxyethylene lauryl ether, glyceryl dilaurate, glyceryl dimystate, glyceryl distearate, semi-synthetic derivatives thereof, or mixtures thereof.

Additional suitable surfactants include, but are not limited to, non-ionic lipids, such as glyceryl laurate, glyceryl myristate, glyceryl dilaurate, glyceryl dimyristate, semi-synthetic derivatives thereof, and mixtures thereof.

In some embodiments, the surfactant is a polyoxyethylene fatty ether having a polyoxyethylene head group ranging from about 2 to about 100 groups, or an alkoxylated alcohol having the structure R₅—(OCH₂ CH₂)_{y}—OH, wherein R₅ is a branched or unbranched alkyl group having from about 6 to about 22 carbon atoms and y is between about 4 and about 100, and preferably, between about 10 and about 100. Preferably, the alkoxylated alcohol is the species wherein R₅ is a lauryl group and y has an average value of 23.

In some embodiments, the surfactant is an alkoxylated alcohol which is an ethoxylated derivative of lanolin alcohol. Preferably, the ethoxylated derivative of lanolin alcohol is laneth-10, which is the polyethylene glycol ether of lanolin alcohol with an average ethoxylation value of 10.

Nonionic surfactants include, but are not limited to, an ethoxylated surfactant, an alcohol ethoxylated, an alkyl phenol ethoxylated, a fatty acid ethoxylated, a monoalkaolamide ethoxylated, a sorbitan ester ethoxylated, a fatty amino ethoxylated, an ethylene oxide-propylene oxide copolymer, Bis(polyethylene glycol bis[imidazoyl carbonyl]), nonoxynol-9, Bis(polyethylene glycol bis[imidazoyl carbonyl]), Brij® 35, Brij® 56, Brij® 72, Brij® 76, Brij® 92V, Brij® 97, Brij® 58P, Cremophor° EL, Decaethylene glycol monododecyl ether, N-Decanoyl-N-methylglucamine, n-Decyl alpha-D-glucopyranoside, Decyl beta-D-maltopyranoside, n-Dodecanoyl-N-methylglucamide, n-Dodecyl alpha-D-maltoside, n-Dodecyl beta-D-maltoside, n-Dodecyl beta-D-maltoside, Heptaethylene glycol monodecyl ether, Heptaethylene glycol monododecyl ether, Heptaethylene glycol monotetradecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Igepal CA-630, Methyl-6-O-(N-heptylcarbamoyl)-alpha-D-glucopyranoside, Nonaethylene glycol monododecyl ether, N-Nonanoyl-N-methylglucamine, N-Nonanoyl-N-methylglucamine, Octaethylene glycol monodecyl ether, Octaethylene glycol monododecyl ether, Octaethylene glycol monohexadecyl ether, Octaethylene glycol monooctadecyl ether, Octaethylene glycol monotetradecyl ether, Octyl-beta-D-glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin from Quillaja bark, Span® 20, Span® 40, Span® 60, Span® 65, Span® 80, Shan® 85, Tergitol, Type 15-S-12, Tergitol, Type 15-S-30, Tergitol, Type 15-S-5, Tergitol, Type 15-S-7, Tergitol, Type 15-S-9, Tergitol, Type NP-10, Tergitol, Type NP-4, Tergitol, Type NP-40, Tergitol, Type NP-7, Tergitol, Type NP-9, Tergitol, Tergitol, Type TMN-10, Tergitol, Type TMN-6, Tetradecyl-beta-D-maltoside, Tetraethylene glycol monodecyl ether, Tetraethylene glycol monododecyl ether, Tetraethylene glycol monotetradecyl ether, Triethylene glycol monodecyl ether, Triethylene glycol monododecyl ether, Triethylene glycol monohexadecyl ether, Triethylene glycol monooctyl ether, Triethylene glycol monotetradecyl ether, Triton CF-21, Triton CF-32, Triton DF-12, Triton DF-16, Triton GR-5M, Triton QS-15, Triton QS-44, Triton X-100, Triton X-102, Triton X-15, Triton X-151, Triton X-200, Triton X-207, Triton® X-100, Triton® X-114, Triton® X-165, Triton® X-305, Triton® X-405, Triton® X-45, Triton® X-705-70, TWEEN® 20, TWEEN® 21, TWEEN® 40, TWEEN® 60, TWEEN® 61, TWEEN® 65, TWEEN® 80, TWEEN® 81, TWEEN° 85, Tyloxapol, n-Undecyl beta-D-glucopyranoside, semi-synthetic derivatives thereof, or combinations thereof.

In addition, the nonionic surfactant can be a poloxamer. Poloxamers are polymers made of a block of polyoxyethylene, followed by a block of polyoxypropylene, followed by a block of polyoxyethylene. The average number of units of polyoxyethylene and polyoxypropylene varies based on the number associated with the polymer. For example, the smallest polymer, Poloxamer 101, consists of a block with an average of 2 units of polyoxyethylene, a block with an average of 16 units of polyoxypropylene, followed by a block with an average of 2 units of polyoxyethylene. Poloxamers range from colorless liquids and pastes to white solids. In cosmetics and personal care products, Poloxamers are used in the formulation of skin cleansers, bath products, shampoos, hair conditioners, mouthwashes, eye makeup remover and other skin and hair products. Examples of Poloxamers include, but are not limited to, Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, Poloxamer 407, Poloxamer 105 Benzoate, and Poloxamer 182 Dibenzoate.

Suitable cationic surfactants include, but are not limited to, a quarternary ammonium compound, an alkyl trimethyl ammonium chloride compound, a dialkyl dimethyl ammonium chloride compound, a cationic halogen-containing compound, such as cetylpyridinium chloride, Benzalkonium chloride, Benzalkonium chloride, Benzyldimethylhexadecylammonium chloride, Benzyldimethyltetradecylammonium chloride, Benzyldodecyldimethylammonium bromide, Benzyltrimethylammonium tetrachloroiodate, Dimethyldioctadecylammonium bromide, Dodecylethyldimethylammonium bromide, Dodecyltrimethylammonium bromide, Dodecyltrimethylammonium bromide, Ethylhexadecyldimethylammonium bromide, Girard's reagent T, Hexadecyltrimethylammonium bromide, Hexadecyltrimethylammonium bromide, N,N′,N′-Polyoxyethylene(10)-N-tallow-1,3-diaminopropane, Thonzonium bromide, Trimethyl(tetradecyl)ammonium bromide, 1,3,5-Triazine-1,3,5(2H,4H,6H)-triethanol, 1-Decanaminium, N-decyl-N, N-dimethyl-, chloride, Didecyl dimethyl ammonium chloride, 2-(2-(p-(Diisobutyl)cresosxy)ethoxy)ethyl dimethyl benzyl ammonium chloride, 2-(2-(p-(Diisobutyl)phenoxy)ethoxy)ethyl dimethyl benzyl ammonium chloride, Alkyl 1 or 3 benzyl-1-(2-hydroxethyl)-2-imidazolinium chloride, Alkyl bis(2-hydroxyethyl) benzyl ammonium chloride, Alkyl demethyl benzyl ammonium chloride, Alkyl dimethyl 3,4-dichlorobenzyl ammonium chloride (100% C₁₂), Alkyl dimethyl 3,4-dichlorobenzyl ammonium chloride (50% C₁₄, 40% C₁₂, 10% C₁₆), Alkyl dimethyl 3,4-dichlorobenzyl ammonium chloride (55% C₁₄, 23% C₁₂, 20% C₁₆), Alkyl dimethyl benzyl ammonium chloride, Alkyl dimethyl benzyl ammonium chloride (100% C₁₄), Alkyl dimethyl benzyl ammonium chloride (100% C₁₆), Alkyl dimethyl benzyl ammonium chloride (41% C₁₄, 28% C₁₂), Alkyl dimethyl benzyl ammonium chloride (47% C₁₂, 18% C₁₄), Alkyl dimethyl benzyl ammonium chloride (55% C16, 20% C14), Alkyl dimethyl benzyl ammonium chloride (58% C₁₄, 28% C₁₆), Alkyl dimethyl benzyl ammonium chloride (60% C₁₄, 25% C₁₂), Alkyl dimethyl benzyl ammonium chloride (61% C₁₁, 23% C₁₄), Alkyl dimethyl benzyl ammonium chloride (61% C₁₂, 23% C₁₄), Alkyl dimethyl benzyl ammonium chloride (65% C₁₂, 25% C₁₄), Alkyl dimethyl benzyl ammonium chloride (67% C₁₂, 24% C₁₄), Alkyl dimethyl benzyl ammonium chloride (67% C₁₂, 25% C₁₄), Alkyl dimethyl benzyl ammonium chloride (90% C₁₄, 5% Cu), Alkyl dimethyl benzyl ammonium chloride (93% C₁₄, 4% Cu), Alkyl dimethyl benzyl ammonium chloride (95% C₁₆, 5% C18), Alkyl dimethyl benzyl ammonium chloride, Alkyl didecyl dimethyl ammonium chloride, Alkyl dimethyl benzyl ammonium chloride, Alkyl dimethyl benzyl ammonium chloride (C₁₂₋₁₆), Alkyl dimethyl benzyl ammonium chloride (C₁₂₋₁₈), Alkyl dimethyl benzyl ammonium chloride, dialkyl dimethyl benzyl ammonium chloride, Alkyl dimethyl dimethybenzyl ammonium chloride, Alkyl dimethyl ethyl ammonium bromide (90% C₁₄, 5% C₁₆, 5% C₁₂), Alkyl dimethyl ethyl ammonium bromide (mixed alkyl and alkenyl groups as in the fatty acids of soybean oil), Alkyl dimethyl ethylbenzyl ammonium chloride, Alkyl dimethyl ethylbenzyl ammonium chloride (60% C₁₄), Alkyl dimethyl isopropylbenzyl ammonium chloride (50% C₁₂, 30% C₁₄, 17% C₁₆, 3% C₁₈), Alkyl trimethyl ammonium chloride (58% C₁₈, 40% C₁₆, 1% C₁₄, 1% C₁₂), Alkyl trimethyl ammonium chloride (90% C₁₈, 10% C₁₆), Alkyldimethyl(ethylbenzyl) ammonium chloride (C₁₂₋₁₈), Di-(C₈₋₁₀)-alkyl dimethyl ammonium chlorides, Dialkyl dimethyl ammonium chloride, Dialkyl methyl benzyl ammonium chloride, Didecyl dimethyl ammonium chloride, Diisodecyl dimethyl ammonium chloride, Dioctyl dimethyl ammonium chloride, Dodecyl bis (2-hydroxyethyl) octyl hydrogen ammonium chloride, Dodecyl dimethyl benzyl ammonium chloride, Dodecylcarbamoyl methyl dinethyl benzyl ammonium chloride, Heptadecyl hydroxyethylimidazolinium chloride, Hexahydro-1,3,5-tris(2-hydroxyethyl)-s-triazine, Hexahydro-1,3,5-tris(2-hydroxyethyl)-s-triazine, Myristalkonium chloride (and) Quat RNIUM 14, N,N-Dimethyl-2-hydroxypropylammonium chloride polymer, n-Tetradecyl dimethyl benzyl ammonium chloride monohydrate, Octyl decyl dimethyl ammonium chloride, Octyl dodecyl dimethyl ammonium chloride, Octyphenoxyethoxyethyl dimethyl benzyl ammonium chloride, Oxydiethylenebis(alkyl dimethyl ammonium chloride), Quaternary ammonium compounds, dicoco alkyldimethyl, chloride, Trimethoxysily propyl dimethyl octadecyl ammonium chloride, Trimethoxysilyl quats, Trimethyl dodecylbenzyl ammonium chloride, semi-synthetic derivatives thereof, and combinations thereof.

Exemplary cationic halogen-containing compounds include, but are not limited to, cetylpyridinium halides, cetyltrimethylammonium halides, cetyldimethylethylammonium halides, cetyldimethylbenzylammonium halides, cetyltributylphosphonium halides, dodecyltrimethylammonium halides, or tetradecyltrimethylammonium halides. In some particular embodiments, suitable cationic halogen containing compounds comprise, but are not limited to, cetylpyridinium chloride (CPC), cetyltrimethylammonium chloride, cetylbenzyldimethylammonium chloride, cetylpyridinium bromide (CPB), cetyltrimethylammonium bromide (CTAB), cetyidimethylethylammonium bromide, cetyltributylphosphonium bromide, dodecyltrimethylammonium bromide, and tetrad ecyltrimethylammonium bromide. In particularly preferred embodiments, the cationic halogen containing compound is CPC, although the compositions of the present invention are not limited to formulation with an particular cationic containing compound.

Suitable anionic surfactants include, but are not limited to, a carboxylate, a sulphate, a sulphonate, a phosphate, chenodeoxycholic acid, chenodeoxycholic acid sodium salt, cholic acid, ox or sheep bile, Dehydrocholic acid, Deoxycholic acid, Deoxycholic acid, Deoxycholic acid methyl ester, Digitonin, Digitoxigenin, N,N-Dimethyldodecylamine N-oxide, Docusate sodium salt, Glycochenodeoxycholic acid sodium salt, Glycocholic acid hydrate, synthetic, Glycocholic acid sodium salt hydrate, synthetic, Glycodeoxycholic acid monohydrate, Glycodeoxycholic acid sodium salt, Glycodeoxycholic acid sodium salt, Glycolithocholic acid 3-sulfate disodium salt, Glycolithocholic acid ethyl ester, N-Lauroylsarcosine sodium salt, N-Lauroylsarcosine solution, N-Lauroylsarcosine solution, Lithium dodecyl sulfate, Lithium dodecyl sulfate, Lithium dodecyl sulfate, Lugol solution, Niaproof 4, Type 4, 1-Octanesulfonic acid sodium salt, Sodium 1-butanesulfonate, Sodium 1-decanesulfonate, Sodium 1-decanesulfonate, Sodium 1-dodecanesulfonate, Sodium 1-heptanesulfonate anhydrous, Sodium 1-heptanesulfonate anhydrous, Sodium 1-nonanesulfonate, Sodium 1-propanesulfonate monohydrate, Sodium 2-bromoethanesulfonate, Sodium cholate hydrate, Sodium choleate, Sodium deoxycholate, Sodium deoxycholate monohydrate, Sodium dodecyl sulfate, Sodium hexanesulfonate anhydrous, Sodium octyl sulfate, Sodium pentanesulfonate anhydrous, Sodium taurocholate, Taurochenodeoxycholic acid sodium salt, Taurodeoxycholic acid sodium salt monohydrate, Taurohyodeoxycholic acid sodium salt hydrate, Taurolithocholic acid 3-sulfate disodium salt, Tauroursodeoxycholic acid sodium salt, Trizma® dodecyl sulfate, TWEEN° 80, Ursodeoxycholic acid, semi-synthetic derivatives thereof, and combinations thereof.

Suitable zwitterionic surfactants include, but are not limited to, an N-alkyl betaine, lauryl amindo propyl dimethyl betaine, an alkyl dimethyl glycinate, an N-alkyl amino propionate, CHAPS, minimum 98% (TLC), CHAPS, SigmaUltra, minimum 98% (TLC), CHAPS, for electrophoresis, minimum 98% (TLC), CHAPSO, minimum 98%, CHAPSO, SigmaUltra, CHAPSO, for electrophoresis, 3-(Decyldimethylammonio)propanesulfonate inner salt, 3-Dodecyldimethylammonio)propanesulfonate inner salt, SigmaUltra, 3-(Dodecyldimethylammonio) propanesulfonate inner salt, 3-(N,N-Dimethylmyristylammonio) propanesulfonate, 3-(N,N-Dimethyloctadecylammonio)propanesulfonate, 3-(N,N-Dimethyloctylammonio)propanesulfonate inner salt, 3-(N,N-Dimethylpalmitylammonio) propanesulfonate, semi-synthetic derivatives thereof, and combinations thereof.

In some embodiments, the nanoemulsion RSV vaccine comprises a cationic surfactant, which can be cetylpyridinium chloride. In other embodiments of the invention, the nanoemulsion RSV vaccine comprises a cationic surfactant, and the concentration of the cationic surfactant is less than about 5.0% and greater than about 0.001%. In yet another embodiment of the invention, the nanoemulsion RSV vaccine comprises a cationic surfactant, and the concentration of the cationic surfactant is selected from the group consisting of less than about 5%, less than about 4.5%, less than about 4.0%, less than about 3.5%, less than about 3.0%, less than about 2.5%, less than about 2.0%, less than about 1.5%, less than about 1.0%, less than about 0.90%, less than about 0.80%, less than about 0.70%, less than about 0.60%, less than about 0.50%, less than about 0.40%, less than about 0.30%, less than about 0.20%, or less than about 0.10%. Further, the concentration of the cationic agent in the nanoemulsion vaccine is greater than about 0.002%, greater than about 0.003%, greater than about 0.004%, greater than about 0.005%, greater than about 0.006%, greater than about 0.007%, greater than about 0.008%, greater than about 0.009%, greater than about 0.010%, or greater than about 0.001%. In one embodiment, the concentration of the cationic agent in the nanoemulsion vaccine is less than about 5.0% and greater than about 0.001%.

In another embodiment of the invention, the nanoemulsion vaccine comprises at least one cationic surfactant and at least one non-cationic surfactant. The non-cationic surfactant is a nonionic surfactant, such as a polysorbate (Tween), such as polysorbate 80 or polysorbate 20. In one embodiment, the non-ionic surfactant is present in a concentration of about 0.01% to about 5.0%, or the non-ionic surfactant is present in a concentration of about 0.1% to about 3%. In yet another embodiment of the invention, the nanoemulsion vaccine comprises a cationic surfactant present in a concentration of about 0.01% to about 2%, in combination with a nonionic surfactant.

f. Additional Ingredients for RSV Vaccine Nanoemulsion

Additional compounds suitable for use in the nanoemulsion RSV vaccines can include but are not limited to one or more solvents, such as an organic phosphate-based solvent, bulking agents, coloring agents, pharmaceutically acceptable excipients, a preservative, pH adjuster, buffer, chelating agent, etc. The additional compounds can be admixed into a previously emulsified nanoemulsion vaccine, or the additional compounds can be added to the original mixture to be emulsified. In certain of these embodiments, one or more additional compounds are admixed into an existing nanoemulsion composition immediately prior to its use.

Suitable preservatives in the nanoemulsion RSV vaccines of the invention include, but are not limited to, cetylpyridinium chloride, benzalkonium chloride, benzyl alcohol, chlorhexidine, imidazolidinyl urea, phenol, potassium sorbate, benzoic acid, bronopol, chlorocresol, paraben esters, phenoxyethanol, sorbic acid, alpha-tocophernol, ascorbic acid, ascorbyl palmitate, butylated hydroxyanisole, butylated hydroxytoluene, sodium ascorbate, sodium metabisulphite, citric acid, edetic acid, semi-synthetic derivatives thereof, and combinations thereof. Other suitable preservatives include, but are not limited to, benzyl alcohol, chlorhexidine (bis (p-chlorophenyldiguanido) hexane), chlorphenesin (3-(-4-chloropheoxy)-propane-1,2-diol), Kathon CG (methyl and methylchloroisothiazolinone), parabens (methyl, ethyl, propyl, butyl hydrobenzoates), phenoxyethanol (2-phenoxyethanol), sorbic acid (potassium sorbate, sorbic acid), Phenonip (phenoxyethanol, methyl, ethyl, butyl, propyl parabens), Phenoroc (phenoxyethanol 0.73%, methyl paraben 0.2%, propyl paraben 0.07%), Liquipar Oil (isopropyl, isobutyl, butylparabens), Liquipar PE (70% phenoxyethanol, 30% liquipar oil), Nipaguard MPA (benzyl alcohol (70%), methyl & propyl parabens), Nipaguard MPS (propylene glycol, methyl & propyl parabens), Nipasept (methyl, ethyl and propyl parabens), Nipastat (methyl, butyl, ethyl and propyel parabens), Elestab 388 (phenoxyethanol in propylene glycol plus chlorphenesin and methylparaben), and Killitol (7.5% chlorphenesin and 7.5% methyl parabens).

The nanoemulsion RSV vaccine may further comprise at least one pH adjuster. Suitable pH adjusters in the nanoemulsion vaccine of the invention include, but are not limited to, diethyanolamine, lactic acid, monoethanolamine, triethylanolamine, sodium hydroxide, sodium phosphate, semi-synthetic derivatives thereof, and combinations thereof.

In addition, the nanoemulsion RSV vaccine can comprise a chelating agent. In one embodiment of the invention, the chelating agent is present in an amount of about 0.0005% to about 1%. Examples of chelating agents include, but are not limited to, ethylenediamine, ethylenediaminetetraacetic acid (EDTA), phytic acid, polyphosphoric acid, citric acid, gluconic acid, acetic acid, lactic acid, and dimercaprol, and a preferred chelating agent is ethylenediaminetetraacetic acid.

The nanoemulsion RSV vaccine can comprise a buffering agent, such as a pharmaceutically acceptable buffering agent. Examples of buffering agents include, but are not limited to, 2-Amino-2-methyl-1,3-propanediol, ≥99.5% (NT), 2-Amino-2-methyl-1-propanol, ≥99.0% (GC), L-(+)-Tartaric acid, ≥99.5% (T), ACES, ≥99.5% (T), ADA, ≥99.0% (T), Acetic acid, ≥99.5% (GC/T), Acetic acid, for luminescence, ≥99.5% (GC/T), Ammonium acetate solution, for molecular biology, ˜5 M in H₂O, Ammonium acetate, for luminescence, ≥99.0% (calc. on dry substance, T), Ammonium bicarbonate, ≥99.5% (T), Ammonium citrate dibasic, ≥99.0% (T), Ammonium formate solution , 10 M in H₂O , Ammonium formate, ≥99.0% (calc. based on dry substance, NT), Ammonium oxalate monohydrate, ≥99.5% (RT), Ammonium phosphate dibasic solution, 2.5 M in H₂O , Ammonium phosphate dibasic, ≥99.0% (T), Ammonium phosphate monobasic solution, 2.5 M in H₂O, Ammonium phosphate monobasic, ≥99.5% (T), Ammonium sodium phosphate dibasic tetrahydrate, ≥99.5% (NT), Ammonium sulfate solution, for molecular biology, 3.2 M in H₂O, Ammonium tartrate dibasic solution , 2 M in H₂O (colorless solution at 20 ° C.), Ammonium tartrate dibasic, ≥99.5% (T), BES buffered saline, for molecular biology, 2× concentrate, BES , ≥99.5% (T), BES, for molecular biology, ≥99.5% (T), BICINE buffer Solution, for molecular biology, 1 M in H₂O, BICINE, ≥99.5% (T), BIS-TRIS, ≥99.0% (NT), Bicarbonate buffer solution , >0.1 M Na₂CO₃, >0.2 M NaHCO₃, Boric acid , ≥99.5% (T), Boric acid, for molecular biology, ≥99.5% (T), CAPS, ≥99.0% (TLC), CHES, ≥99.5% (T), Calcium acetate hydrate, ≥99.0% (calc. on dried material, KT), Calcium carbonate, precipitated, ≥99.0% (KT), Calcium citrate tribasic tetrahydrate, ≥98.0% (calc. on dry substance, KT), Citrate Concentrated Solution, for molecular biology, 1 M in H₂O Citric acid , anhydrous, ≥99.5% (T), Citric acid , for luminescence, anhydrous, ≥99.5% (T), Diethanolamine, ≥99.5% (GC), EPPS , ≥99.0% (T), Ethylenediaminetetraacetic acid disodium salt dihydrate, for molecular biology, ≥99.0% (T), Formic acid solution , 1.0 M in H₂O, Gly-Gly-Gly, ≥99.0% (NT), Gly-Gly, ≥99.5% (NT), Glycine, ≥99.0% (NT), Glycine, for luminescence, ≥99.0% (NT), Glycine, for molecular biology, ≥99.0% (NT), HEPES buffered saline, for molecular biology, 2× concentrate, HEPES , ≥99.5% (T), HEPES, for molecular biology, ≥99.5% (T), Imidazole buffer Solution, 1 M in H₂O, Imidazole, ≥99.5% (GC), Imidazole, for luminescence, ≥99.5% (GC), Imidazole, for molecular biology, ≥99.5% (GC), Lipoprotein Refolding Buffer, Lithium acetate dihydrate, ≥99.0% (NT), Lithium citrate tribasic tetrahydrate, ≥99.5% (NT), MES h_{y}drate, ≥99.5% (T), MES monohydrate, for luminescence, ≥99.5% (T), MES solution, for molecular biology, 0.5 M in H₂O, MOPS, ≥99.5% (T), MOPS, for luminescence, ≥99.5% (T), MOPS, for molecular biology, ≥99.5% (T), Magnesium acetate solution, for molecular biology, ˜1 M in H₂O, Magnesium acetate tetrahydrate, ≥99.0% (KT), Magnesium citrate tribasic nonahydrate, ≥98.0% (calc. based on dry substance, KT), Magnesium formate solution, 0.5 M in H₂O, Magnesium phosphate dibasic trihydrate, ≥98.0% (KT), Neutralization solution for the in-situ hybridization for in-situ hybridization, for molecular biology, Oxalic acid dihydrate, ≥99.5% (RT), PIPES, ≥99.5% (T), PIPES, for molecular biology, ≥99.5% (T), Phosphate buffered saline, solution (autoclaved), Phosphate buffered saline, washing buffer for peroxidase conjugates in Western Blotting, 10× concentrate, Piperazine, anhydrous, ≥99.0% (T), Potassium D-tartrate monobasic , ≥99.0% (T), Potassium acetate solution , for molecular biology, Potassium acetate solution, for molecular biology, 5 M in H₂O, Potassium acetate solution, for molecular biology, ˜1 M in H₂O, Potassium acetate, ≥99.0% (NT), Potassium acetate, for luminescence, ≥99.0% (NT), Potassium acetate, for molecular biology, ≥99.0% (NT), Potassium bicarbonate , ≥99.5% (T), Potassium carbonate , anhydrous, ≥99.0% (T), Potassium chloride, ≥99.5% (AT), Potassium citrate monobasic , ≥99.0% (dried material, NT), Potassium citrate tribasic solution , 1 M in H₂O, Potassium formate solution , 14 M in H₂O, Potassium formate , ≥99.5% (NT), Potassium oxalate monohydrate, ≥99.0% (RT), Potassium phosphate dibasic, anhydrous, ≥99.0% (T), Potassium phosphate dibasic, for luminescence, anhydrous, ≥99.0% (T), Potassium phosphate dibasic, for molecular biology, anhydrous, ≥99.0% (T), Potassium phosphate monobasic, anhydrous, ≥99.5% (T), Potassium phosphate monobasic, for molecular biology, anhydrous, ≥99.5% (T), Potassium phosphate tribasic monohydrate, ≥95% (T), Potassium phthalate monobasic, ≥99.5% (T), Potassium sodium tartrate solution, 1.5 M in H₂O, Potassium sodium tartrate tetrahydrate, ≥99.5% (NT), Potassium tetraborate tetrahydrate, ≥99.0% (T), Potassium tetraoxalate dihydrate, ≥99.5% (RT), Propionic acid solution, 1.0 M in H₂O, STE buffer solution, for molecular biology, pH 7.8, STET buffer solution, for molecular biology, pH 8.0, Sodium 5,5-diethylbarbiturate , ≥99.5% (NT), Sodium acetate solution, for molecular biology, ˜3 M in H₂O, Sodium acetate trihydrate, ≥99.5% (NT), Sodium acetate, anhydrous, ≥99.0% (NT), Sodium acetate, for luminescence, anhydrous, ≥99.0% (NT), Sodium acetate, for molecular biology, anhydrous, ≥99.0% (NT), Sodium bicarbonate, ≥99.5% (T), Sodium bitartrate monohydrate, ≥99.0% (T), Sodium carbonate decahydrate, ≥99.5% (T), Sodium carbonate, anhydrous, ≥99.5% (calc. on dry substance, T), Sodium citrate monobasic, anhydrous, ≥99.5% (T), Sodium citrate tribasic dihydrate, ≥99.0% (NT), Sodium citrate tribasic dihydrate, for luminescence, ≥99.0% (NT), Sodium citrate tribasic dihydrate, for molecular biology, ≥99.5% (NT), Sodium formate solution, 8 M in H₂O, Sodium oxalate, ≥99.5% (RT), Sodium phosphate dibasic dihydrate, ≥99.0% (T), Sodium phosphate dibasic dihydrate, for luminescence, ≥99.0% (T), Sodium phosphate dibasic dihydrate , for molecular biology, ≥99.0% (T), Sodium phosphate dibasic dodecahydrate, ≥99.0% (T), Sodium phosphate dibasic solution, 0.5 M in H₂O, Sodium phosphate dibasic, anhydrous, ≥99.5% (T), Sodium phosphate dibasic , for molecular biology, ≥99.5% (T), Sodium phosphate monobasic dihydrate, ≥99.0% (T), Sodium phosphate monobasic dihydrate, for molecular biology, ≥99.0% (T), Sodium phosphate monobasic monohydrate , for molecular biology, ≥99.5% (T), Sodium phosphate monobasic solution, 5 M in H₂O, Sodium pyrophosphate dibasic, ≥99.0% (T), Sodium pyrophosphate tetrabasic decahydrate, ≥99.5% (T), Sodium tartrate dibasic dihydrate, ≥99.0% (NT), Sodium tartrate dibasic solution , 1.5 M in H20 (colorless solution at 20 ° C.), Sodium tetraborate decahydrate , ≥99.5% (T), TAPS , ≥99.5% (T), TES, ≥99.5% (calc. based on dry substance, T), TM buffer solution, for molecular biology, pH 7.4, TNT buffer solution, for molecular biology, pH 8.0, TRIS Glycine buffer solution, 10× concentrate, TRIS acetate - EDTA buffer solution, for molecular biology, TRIS buffered saline, 10× concentrate, TRIS glycine SDS buffer solution, for electrophoresis, 10× concentrate, TRIS phosphate-EDTA buffer solution, for molecular biology, concentrate, 10× concentrate, Tricine, ≥99.5% (NT), Triethanolamine, ≥99.5% (GC), Triethylamine, ≥99.5% (GC), Triethylammonium acetate buffer, volatile buffer, ˜1.0 M in H₂O, Triethylammonium phosphate solution, volatile buffer, ˜1.0 M in H₂O, Trimethylammonium acetate solution, volatile buffer, ˜1.0 M in H₂O, Trimethylammonium phosphate solution, volatile buffer, ˜1 M in H₂O, Tris-EDTA buffer solution, for molecular biology, concentrate, 100× concentrate, Tris-EDTA buffer solution , for molecular biology, pH 7.4, Tris-EDTA buffer solution, for molecular biology, pH 8.0, Trizma® acetate, ≥99.0% (NT), Trizma® base , ≥99.8% (T), Trizma® base, ≥99.8% (T), Trizma® base , for luminescence, ≥99.8% (T), Trizma® base, for molecular biology, ≥99.8% (T), Trizma® carbonate, ≥98.5% (T), Trizma® hydrochloride buffer solution, for molecular biology, pH 7.2, Trizma® hydrochloride buffer solution, for molecular biology, pH 7.4, Trizma® hydrochloride buffer solution, for molecular biology, pH 7.6, Trizma® hydrochloride buffer solution , for molecular biology, pH 8.0, Trizma® hydrochloride, ≥99.0% (AT), Trizma® hydrochloride , for luminescence, ≥99.0% (AT), Trizma® hydrochloride, for molecular biology, ≥99.0% (AT), and Trizma® maleate, ≥99.5% (NT).

The nanoemulsion RSV vaccine can comprise one or more emulsifying agents to aid in the formation of emulsions. Emulsifying agents include compounds that aggregate at the oil/water interface to form a kind of continuous membrane that prevents direct contact between two adjacent droplets. Certain embodiments of the present invention feature nanoemulsion vaccines that may readily be diluted with water or another aqueous phase to a desired concentration without impairing their desired properties.

g. Immune Modulators for RSV Vaccine Nanoemulsion

As noted above, the RSV vaccine can further comprise one or more immune modulators. Examples of immune modulators include, but are not limited to, chitosan, glucan, enterotoxin, nucleic acid (CpG motifs), MF59, alum, ASO system, etc. It is within the purview of one of ordinary skill in the art to employ suitable immune modulators in the context of the present invention.

An immune modulator can be present in the vaccine composition at any pharmaceutically acceptable amount including, but not limited to, from about 0.001% up to about 10%, and any amount inbetween, such as about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10%.

4. Pharmaceutical Compositions of RSV Vaccines

The nanoemulsion RSV vaccines may be formulated into pharmaceutical compositions that comprise the nanoemulsion RSV vaccine in a therapeutically effective amount and suitable, pharmaceutically-acceptable excipients for pharmaceutically acceptable delivery. Such excipients are well known in the art.

In the context of immunization of the donor, the phrase “therapeutically effective amount” means any amount of the nanoemulsion RSV vaccine that is effective in eliciting an immune response that comprises production of antibodies that specifically recognize and/or bind to RSV. By “protective immune response” it is meant that the immune response is associated with prevention, treating, or amelioration of a disease. Complete prevention is not required, though is encompassed by the present invention. The immune response can be evaluated using the methods discussed herein or by any method known by a person of skill in the art.

Intranasal administration includes administration via the nose, either with or without concomitant inhalation during administration. Such administration is typically through contact by the composition comprising the nanoemulsion RSV vaccine with the nasal mucosa, nasal turbinates or sinus cavity. Administration by inhalation comprises intranasal administration, or may include oral inhalation. Such administration may also include contact with the oral mucosa, bronchial mucosa, and other epithelia. Intramuscular administration is another acceptable route of administration and comprises injection of a compound directly into the muscle of a subject.

The pharmaceutical nanoemulsion RSV vaccines for administration may be applied in a single administration or in multiple administrations.

An exemplary nanoemulsion adjuvant composition according to the invention is designated “W₈₀5EC” adjuvant. The composition of W₈₀5EC adjuvant is shown in the table below (Table 1). The mean droplet size for the W₈₀5EC adjuvant is ˜400nm. All of the components of the nanoemulsion are included on the FDA inactive ingredient list for Approved Drug Products.

TABLE 1
W₈₀5EC Formulation

| | W₈₀5EC-Adjuvant |
|---|---|
| Function | Mean Droplet Size ≈400 nm |
| Aqueous Diluent | Purified Water, USP |
| Hydrophobic Oil | Soybean Oil, USP (super refined) |
| (Core) | |
| Organic Solvent | Dehydrated Alcohol, USP (anhydrous |
| | ethanol) |
| Surfactant | Polysorbate 80, NF |
| Emulsifying Agent | Cetylpyridinium Chloride, USP |
| Preservative | |

A nanoemulsion as provided herein (e.g. W₈₀5EC or DODAC/CPC NE) can make up between about 1% -about 99% (w/w %) of an injectable composition (e.g., an immunogenic composition (e.g., a vaccine)) of the invention. For instance, the nanoemulsion can be about 1, μg, 5, about 10, about 15, about 20, about 25, about 30, about 35, about 40, about 45, about 50, about 55, about 60, about 65, about 70, about 75, about 80, about 85, about 90, about 95, or about 99% of an injectable composition (e.g., an immunogenic composition (e.g., a vaccine)) of the invention.

The nanoemulsion adjuvants are formed by emulsification of an oil, purified water, nonionic detergent, organic solvent and surfactant, such as a cationic surfactant. An exemplary specific nanoemulsion adjuvant is designated as “60%W₈₀5EC”. The 60%W₈₀5EC-adjuvant is composed of the ingredients shown in Table 2 below: purified water, USP; soybean oil USP; Dehydrated Alcohol, USP [anhydrous ethanol]; Polysorbate 80, NF and cetylpyridinium chloride, USP (CPC). All components of this exemplary nanoemulsion are included on the FDA list of approved inactive ingredients for Approved Drug Products.

TABLE 2
Composition of 60% W₈₀5EC-NanoEmulsion Adjuvant (w/w %)

| Ingredients | 60% W₈₀5EC |
|---|---|
| Purified Water, USP | 54.10% |
| Soybean Oil, USP | 37.67% |
| Dehydrated Alcohol, USP | 4.04% |
| (anhydrous ethanol) | |
| Polysorbate 80, NF | 3.55% |
| Cetylpyridinium Chloride, USP | 0.64% |

5. Methods of Manufacture of Nanoemulsions

The nanoemulsions for use in the RSV vaccines disclosed herein can be formed using classic emulsion forming techniques. See e.g., U.S. 2004-0043041. In an exemplary method, the oil is mixed with the aqueous phase under relatively high shear forces (e.g., using high hydraulic and mechanical forces) to obtain a nanoemulsion comprising oil droplets having an average diameter of less than about 1000 nm. Some embodiments of the invention employ a nanoemulsion having an oil phase comprising an alcohol such as ethanol. The oil and aqueous phases can be blended using any apparatus capable of producing shear forces sufficient to form an emulsion, such as French Presses or high shear mixers (e.g., FDA approved high shear mixers are available, for example, from Admix, Inc., Manchester, N.H.). Methods of producing such emulsions are described in U.S. Pat. Nos. 5,103,497 and 4,895,452.

In an exemplary embodiment, the nanoemulsions used in the disclosed methods of producing an anti-RSV IVIG can comprise droplets of an oily discontinuous phase dispersed in an aqueous continuous phase, such as water or PBS. The nanoemulsions are stable, and do not deteriorate even after long storage periods. Certain nanoemulsions are non-toxic and safe when swallowed, inhaled, or contacted to the skin of a subject or donor.

The nanoemulsion RSV vaccines disclosed above can be produced in large quantities and are stable for many months at a broad range of temperatures. The nanoemulsion can have textures ranging from that of a semi-solid cream to that of a thin lotion, to that of a liquid and can be applied topically by any pharmaceutically acceptable method as stated above, e.g., by hand, or nasal drops/spray.

As stated above, at least a portion of the emulsion may be in the form of lipid structures including, but not limited to, unilamellar, multilamellar, and paucliamellar lipid vesicles, micelles, and lamellar phases.

The present disclosure contemplates that many variations of the described nanoemulsions will be useful in the methods of the present invention. To determine if a candidate nanoemulsion is suitable for use with the present invention, three criteria are analyzed. Using the methods and standards described herein, candidate emulsions can be easily tested to determine if they are suitable for producing IVIG. First, the desired ingredients are prepared using the methods described herein, to determine if a nanoemulsion can be formed. If a nanoemulsion cannot be formed, the candidate is rejected. Second, the candidate nanoemulsion should form a stable emulsion. A nanoemulsion is stable if it remains in emulsion form for a sufficient period to allow its intended use. For example, for nanoemulsions that are to be stored, shipped, etc., it may be desired that the nanoemulsion remain in emulsion form for months to years. Typical nanoemulsions that are relatively unstable, will lose their form within a day. Third, the candidate nanoemulsion should have efficacy for its intended use. For example, the emulsions should kill or disable RSV virus to a detectable level, or induce a protective immune response to a detectable level. The nanoemulsion can be provided in many different types of containers and delivery systems. For example, in some embodiments, the nanoemulsions are provided in a cream or other solid or semi-solid form. The nanoemulsions may be incorporated into hydrogel formulations.

The nanoemulsions can be delivered (e.g., to a subject or donor) in any suitable container. Suitable containers can be used that provide one or more single use or multi-use dosages of the nanoemulsion for the desired application. In some embodiments, the nanoemulsions are provided in a suspension or liquid form. Such nanoemulsions can be delivered in any suitable container including spray bottles and any suitable pressurized spray device. Such spray bottles may be suitable for delivering the nanoemulsions intranasally or via inhalation.

These nanoemulsion-containing containers can further be packaged with instructions for use to form kits.

### IV. Definitions

As used herein, “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

As used herein, the term “adjuvant” refers to an agent that increases the immune response to an antigen (e.g., a pathogen). As used herein, the term “immune response” refers to a subject's (e.g., a human or another animal) response by the immune system to immunogens (i.e., antigens) which the subject's immune system recognizes as foreign. Immune responses include both cell-mediated immune responses (responses mediated by antigen-specific T cells and non-specific cells of the immune system) and humoral immune responses (responses mediated by antibodies present in the plasma lymph, and tissue fluids). The term “immune response” encompasses both the initial responses to an immunogen (e.g., a pathogen) as well as memory responses that are a result of “acquired immunity.”

As used herein, the term “attenuated” RSV refers to viral particles with reduced virulence and pathogenicity and in animal models and human will not result in clinical diseases.

As used herein, the term “subunit” refers to isolated and generally purified RSV glycoproteins that are individually or mixed further with nanoemulsion comprising a vaccine composition. The subunit vaccine composition is free from mature virions, cells or lysate of cell or virions. The method of obtaining a viral surface antigen that is included in a subunit vaccine can be conducted using standard recombinant genetics techniques and synthetic methods and with standard purification protocols.

The terms “chelator” or “chelating agent” refer to any materials having more than one atom with a lone pair of electrons that are available to bond to a metal ion.

As used herein, the term “enhanced immunity” refers to an increase in the level of acquired immunity to a given pathogen following administration of a vaccine of the present invention relative to the level of acquired immunity when a vaccine of the present invention has not been administered.

As used herein, the term “hyperproducer” refers to a viral strain that is capable of selectively producing at least 2-fold higher levels of viral structural proteins over standard viral strains. In the preferred embodiment, hyperproducer refers to the unique demonstration that RSV-L19 produces levels of F and G proteins that are considerably higher than the comparable A2 RSV strain.

As used herein, the term “immunogen” refers to an antigen that is capable of eliciting an immune response in a subject. In preferred embodiments, immunogens elicit immunity against the immunogen (e.g., a pathogen or a pathogen product) when administered in combination with a nanoemulsion of the present invention.

As used herein, the term “inactivated” RSV refers to virion particles that are incapable of infecting host cells and are noninfectious in pertinent animal models.

As used herein, the term “intranasal(ly)” refers to application of the compositions of the present invention to the surface of the skin and mucosal cells and tissues of the nasal passages, e.g., nasal mucosa, sinus cavity, nasal turbinates, or other tissues and cells which line the nasal passages.

The term “nanoemulsion,” as used herein, includes small oil-in-water dispersions or droplets, as well as other lipid structures which can form as a result of hydrophobic forces which drive apolar residues (i.e., long hydrocarbon chains) away from water and drive polar head groups toward water, when a water immiscible oily phase is mixed with an aqueous phase. These other lipid structures include, but are not limited to, unilamellar, paucilamellar, and multilamellar lipid vesicles, micelles, and lamellar phases. The present invention contemplates that one skilled in the art will appreciate this distinction when necessary for understanding the specific embodiments herein disclosed.

The terms “pharmaceutically acceptable” or “pharmacologically acceptable,” as used herein, refer to compositions that do not substantially produce adverse allergic or adverse immunological reactions when administered to a host (e.g., an animal or a human). Such formulations include any pharmaceutically acceptable dosage form. Examples of such pharmaceutically acceptable dosage forms include, but are not limited to, dips, sprays, seed dressings, stem injections, lyophilized dosage forms, sprays, and mists. As used herein, “pharmaceutically acceptable carrier” includes any and all solvents, dispersion media, coatings, wetting agents (e.g., sodium lauryl sulfate), isotonic and absorption delaying agents, disintegrants (e.g., potato starch or sodium starch glycolate), and the like.

As used herein, the phrase “therapeutically effective amount” refers to an amount of the agent that is sufficient to effectuate a desired therapeutic effect on a given condition or disease, e.g., an amount effective to reduce RSV viral load or to lessen, ameliorate, or terminate at least one sign or symptom of RSV infection. Such amount can vary depending on the particular agent, the effect to be achieved, the mode of administration, etc.

As used herein, “viral particles” refers to mature virions, partial virions, empty capsids, defective interfering particles, and viral envelopes.

As used herein, “antibody” includes reference to an immunoglobulin molecule immunologically reactive with a particular antigen, and includes both polyclonal and monoclonal antibodies.

The phrase “specifically binds” when referring to an antibody refers to an antibody that binds RSV with higher affinity than other potential antigens. For example, under designated immunoassay conditions, a specified antibody may selectively bind to RSV with at least two times greater affinity than to background and more typically more than 10 to 100 times background. Specific binding to an antibody under such conditions requires an antibody that is selected for its specificity for a particular protein or antigen that is part of RSV. For example, polyclonal antibodies can be selected to obtain only those polyclonal antibodies that are specifically immunoreactive with RSV and not with other proteins. This selection may be achieved by subtracting out antibodies that cross-react with other molecules. A variety of immunoassay formats are known in the art that may be used to select antibodies specifically immunoreactive for RSV.

As used herein the phrase “intravenous immunoglobulin” or “IVIG” refers to gamma globulin preparations suitable for intravenous use. Typically an IVIG product is prepared from crude plasma or from a crude plasma protein fraction obtained from human normal immunoglobulin (HNI), as described in U.S. Pat. No. 7,138,120.

In the present specification, the term “immunoglobulin-containing plasma fraction” is to encompass all possible starting materials for the present process, e.g. cryoprecipitate-free plasma or cryoprecipitate-free plasma from which various plasma proteins, such as Factor IX and Antithrombin, have been removed, different Cohn fractions, and fractions obtained through precipitation procedures by PEG (Poison et al., (1964), Biochem Biophys Acta, 82, 463-475; Polson and Ruiz-Bravo, (1972) Vox Sang, 23, 107-118) or by ammonium sulphate. In a preferred embodiment, the plasma protein fraction is Cohn fractions II and III, but Cohn fraction II, or Cohn fractions I, II and III can be used as well. The different Cohn fractions are preferably prepared from plasma by a standard Cohn-fractionation method essentially as modified by Kistler-Nitschmann. In addition to immunoglobulins, the Cohn fractions contain e.g. fibrinogen, α-globulins and β-globulins, including various lipoproteins, which should preferably be removed during the subsequent purification process.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein or nucleic acid that is the predominant species present in a preparation is substantially purified. The term “purified” in some embodiments denotes that an antibody or mixture of antibodies is substantially free of other biological material. For example, a purified antibody composition may comprise 85, 85, 87, 88, 89, 90, 91, 92, 93, 94, 95, 96, 97, 98, 99, or 100% antibody, in relation to other biological components in the composition. “Purify” or “purification” in other embodiments means removing at least one contaminant from the composition to be purified. In this sense, purification does not require that the purified compound be homogenous, e.g., 100% pure.

The invention is further described by reference to the following examples, which are provided for illustration only. The invention is not limited to the examples, but rather includes all variations that are evident from the teachings provided herein. All publicly available documents referenced herein, including but not limited to U.S. patents, are specifically incorporated by reference.

### EXAMPLES

### Example 1

### Comparison of RSV Protein Expression

The purpose of this example was to compare expression of F and G proteins from different strains of RSV.

Materials and Methods: All samples were prepared by infecting HEP-2 cells with the same amount of pfu from either A2 or L19 viruses. Twenty four hours post infection, the infected cells were treated with either one of the following:

(1) Cell lysate to check for the cell associated proteins; after discarding the supernatant media, the cells were treated with SDS. This cell lysate was assayed for quantity of F protein associated with the cells.

(2) Total cell and supernatant proteins; the cells and supernatant were frozen and thawed 3 times to lyse the cells and all the cell lysate was used to assay the F protein in the cells and the media.

RSV L19 and RSV A2 virus was extracted and purified from HEP-2 infected cells 4 days following infection. Purified virus was compared for protein contents.

Results: Normalized samples were assayed in Western blots using polyclonal anti RSV antibodies. F and G protein contents were compared between RSV L19 and RSV A2 strains. The density of the bands was compared using image capturing and a Kodak software. A mock non-infected cell culture was prepared as a control.

The results data are detailed in Tables 3-5. Table 3 shows comparable RSV F and G protein from RSV L19 and RSV A2 levels from SDS-PAGE. Table 4 shows comparable RSV L19 and RSV A2 F and G protein from infected cells (Lysate, Supernatant). Finally, Table 5 shows comparable RSV L19 and RSV A2 F and G protein from SDS PAGE.

TABLE 3
Comparable RSV F and G Protein
from RSV L19 and RSV A2 Levels from SDS-PAGE

| | | Infected | Infected | Band Density |
|---|---|---|---|---|
| | Mock | with A2 | with L19 | Ratio (L19/A2) |
| G (90 kDa) | No Band | 78241.1 | 356946.3 | 4.56 |
| F2 (44-45 kDa) | No Band | 38612 | 121328 | 3.14 |

TABLE 4
Comparable RSV L19 and RSV A2 F and G
Protein from Infected Cells (Lysate, Supernatant)

| | Lysate | | | Supernatant | | |
|---|---|---|---|---|---|---|
| | | Infected | Infected | | Infected | Infected |
| | Mock | with A2 | with L19 | Mock | with A2 | with L19 |
| G (90 | No | 27831 | 166308 | 0 | 4686 | 54142 |
| kDa) | Band | | | | | |
| Ratio of | | | 6 | | | 11.6 |
| L19:A2 | | | | | | |
| F2 (44-45 | No | 10645 | 43570 | No | 1860 | 18499 |
| kDa) | Band | | | Band | | |
| Ratio of | | | 4.1 | | | 9.9 |
| L19:A2 | | | | | | |

TABLE 5
Comparable RSV L19 and RSV A2 F
and G Protein From SDS PAGE

| | A2 | L19 | |
|---|---|---|---|
| | (2.3 × 10⁶ pfu) | (2 × 10⁶ pfu) | Ratio (L19/A2) |
| G | 5,039.1 | 11,401.1 | 2.26 |
| F0 + F2 | 4,481.81 | 9,700.39 | 2.16 |

Summary: RSV L19 virus-infected cells produce between 3-11 fold higher quantities of RSV viral proteins as compared to RSV A2 infected cells.

### Example 2

### RSV Inactivation

The purpose of this example was to compare a nanoemulsion adjuvant-inactivated RSV vaccine with a β-ropiolactone (β-PL) inactivated RSV vaccine.

Methods: W₈₀5EC, an oil-in-water nanoemulsion with both antiviral and adjuvant activity, was compared with β-propiolactone (β-PL) inactivated virus (strain L19 @2×105 pfu /dose). The components of the W₈₀5EC nanoemulsion are described in Table 6. The two vaccines were administered intranasally (IN) to BALB/C mice at weeks 0 and 4. Mice were bled prior to dosing and at 3 weeks post-boost and then tested for specific antibodies against F-protein. Inactivation of the RSV (at 2×10⁵ pfu/dose) by W₈₀5EC was achived by incubation of the virus with 20% nanoemulsion (final concentration) for 4 hours at room temperature. β-PL inactivation was achieved by a incubation of the virus with 0.025% β-PL (final concentration) for 16 hours at 4° C.

Animals were challenged nasally with 1×10⁵ pfu RSV L19 at week 8 and checked for airway hyper-reactivity (AHR), lung cytokines, and viral protein mRNA clearance using PCR.

Results: Both W₈₀5EC and β-PL completely inactivated RSV and induced an immune response. β-PL vaccine induced a higher antibody response as compared to nanoemulsion—inactivated RSV vaccine (p=0.006). Animals vaccinated with nanoemulsion—inactivated RSV vaccine, however, had higher clearance of the RSV following the challenge, evidenced by lower proteins F and G mRNA in the lungs (p=0.06 and 0.0004, respectively). Moreover, animals receiving nanoemulsion—inactivated vaccine demonstrated a significant lower AHR (p=0.02). Both RSV vaccines induced significant levels of lung IL-17 as compared to nonvaccinated control (<0.01), however, significantly higher levels were induced by nanoemulsion-inactivated vaccine (p=0.009).

Conclusions: β-PL inactivated RSV virus vaccine is associated with AHR following viral challenge in a mouse model of RSV infection. In contrast, nanoemulsion viral inactivation produced no AHR and induced a significantly increased IL-17 production and improved viral clearance.

### Example 3

### Exemplary Nanoemulsion Vaccines

A total of 10 nanoemulsion formulations were evaluated in mice and cotton rats. W₈₀5EC alone, six W₈₀5EC+Poloxamer 407 and Poloxamer 188 (P407 and P188) formulations as well as two W₈₀5EC+Chitosan and one W₈₀5EC+Glucan formulation have been produced and assessed for stability over 2 weeks under accelerated conditions at 40° C. (Table 6). All 10 nanoemulsions were stable for at least 2 weeks at 40° C.

TABLE 6
W₈₀5EC Formulations

| | | Method of | Particle | Zeta | |
|---|---|---|---|---|---|
| Nanoemulsion | Ratios: | Addition of | Size | Potential | |
| (lot) | CPC:Tween:Poloxamer | Poloxamer | (nm) | (mV) | pH |
| W₈₀5EC | 1:6 | — | 450 | 60 | 4.9 |
| W₈₀5EC + 3% P407 | 1:6 | External | 500 | 56 | 5.9 |
| (AX1e-221-21-03) | | | | | |
| W₈₀5EC/P407 | 1:5:1 | Internal | 391 | 46 | 5.5 |
| (AC1e-221-02) | | | | | |
| W₈₀5EC/P407 | 1:1:5 | Internal | 253 | 36 | 5.2 |
| (AC1e-221-01) | | | | | |
| W₈₀5EC/P188 | 1:5:1 | Internal | 526 | 54 | 5.1 |
| (AO1e-221-04) | | | | | |
| W₈₀5EC/P188 | 1:3:3 | Internal | 416 | 54 | 5.7 |
| (AO1e-221-34) | | | | | |
| W₈₀5EC/P188 | 1:1:5 | Internal | 370 | 47 | 5.2 |
| (AO1e-221-03) | | | | | |
| W₈₀5EC + 0.3% Chitosan | 1:6 | External | 505 | 60 | 5.7 |
| LMW | | | | | |
| (AX1e-221-23-3) | | | | | |
| W₈₀5EC + 0.3% Chitosan | 1:6 | External | 523 | 60 | 5.4 |
| MMW | | | | | |
| (AX1e-221-3-04) | | | | | |
| W₈₀5EC + 0.03% β(1,3) Glucan | 1:6 | External | 491 | 41 | 6.3 |
| (AX1e-221-23-02) | | | | | |

The following formulations were specifically tested in cotton rat IN studies: (1) Formulation 1, W₈₀5EC (NE80), comprising: (a) CPC/Tween 80 (ratio of 1:6), and (b) Particle size ˜500 nm (Table 7); and Formulation 2, W₈₀P₁₈₈5EC (NE188), comprising: (a) CPC/Tween 80/P188 (ratio of 1:1:5), (b) Particle size ˜300 nm, (c) enhanced mucoadhesiveness (IN), and (d) enhanced residence time (IM) (Table 8).

TABLE 7
Formulation 1
Composition of 60% W₈₀5EC adjuvant

| Ingredient | w/w % |
|---|---|
| Distilled water | 54.1 |
| CPC | 0.64 |
| Tween 80 | 3.55 |
| Ethanol | 4.04 |
| Soybean oil | 37.7 |

TABLE 8
Formulation 2
Composition of 60% W₈₀P₁₈₈5EC adjuvant

| Ingredient | w/w % |
|---|---|
| Distilled water | 54.1 |
| CPC | 0.64 |
| Tween 80 | 0.6 |
| Poloxamer 188 | 3 |
| Ethanol | 4.03 |
| Soybean oil | 37.7 |

### Example 4

### RSV Strains for Use in Vaccines

RSV L19 strain was obtained to test as an antigen in the nanoemulsion inactivated/nanoemulsion adjuvanted RSV vaccine. This strain was found to cause infection and enhanced respiratory disease (ERD) in mice. Moreover, published data showed that it conferred protection without induction of ERD in mice when formulated with nanoemulsion. This RSV L19 strain was compared to an RSV wildtype A2 strain obtained from the American Type Culture Collection (ATCC), deposit number PTA-12106.

RSV L19 viral strain is unique in that it produces significantly higher yields of F protein (approximately 10-30 fold more per PFU) than other RSV strains. F protein content may be a key factor in immunogenicity and the RSV L19 strain currently elicits the most robust immune response. The RSV L19 strain has a shorter propagation time and therefore will be more efficient from a manufacturing perspective. RSV L19 strain virus was used for a vaccine in a qualified Vero cell line following single plaque isolation of the L19 strain and purification of the virus to establish a Master Viral Seed Bank and Working Viral Seed Bank. The results comparing the three viral strains are provided in Table 9.

TABLE 9
Comparison of RSV Strains

| | Days of | RSV F | RSV G | | |
|---|---|---|---|---|---|
| | Propaga- | protein | protein | G/F | Viral Titer |
| RSV Strain | tion | (μg/mL) | (μg/mL) | Ratio | (PFU/mL) |
| L 19 | 4-5 | 110 | 603 | 5.5 | 0.5 × 10⁷ |
| A2 Wild Type¹ | 4-5 | 44 | 108 | 2.5 | 1.9 × 10⁷ |
| rA2cp248/404² | 8-9 | 38 | 284 | 7.5 | 0.5 × 10⁸ |

¹ATCC (strain number VR-1540). Virus was isolated from an RSV infected infant with respiratory illness in Melbourne, Australia in 1961 and has been propagated in HEp-2 cell culture at least 27 times (Lewis et al., Med. J. Aust., 2 (1961), pp. 932-933). This virus has been treated to remove adenovirus from the original deposit and has been utilized as a challenge strain in human clinical trials (Lee et al., Antiviral Res. 2004 September; 63(3): 191-6.).
²Recombinant temperature-sensitive A2 mutant virus obtained from the NIH (Whitehead et al., J Virol. 1998; 72: 4467-4471).

### Example 5

### Testing Subunit RSV Vaccines for Immune Response in Mice

The purpose of this example was to evaluate the immunogenic potential, e.g., protective immunity to RSV, of a nanoemulsion-based recombinant F-protein vaccine, comprising W₈₀5EC (adjuvant) and recombinant F protein, in BALB/c mice. The rationale for the example was that using recombinant protein as opposed to killed viral preparations potentially offers numerous advantages in regards to consistency, safety, and manufacturing.

Animals were divided randomly into three groups. Groups were immunized on day 0 and boosted on day 28 intranasally (into nares, half volume per nare). Animals were bled prior to prime immunization and then every 2 weeks throughout the duration of the study. To examine whether vaccination with NE-F protein would affect viral clearance and immunopathology, mice were then challenged with live, infectious RSV intranasally (10⁵ PFU) 2 weeks following the boost immunization.

Test materials: (1) 60% W₈₀5EC, diluted to a final concentration of 20%. The components of W₈₀5EC are shown in Table 10 below.

TABLE 10
W₈₀5EC Formulation

| | W₈₀5EC-Adjuvant; Mean Droplet |
|---|---|
| Function | Size ≈400 nm |
| Aqueous Diluent | Purified Water, USP |
| Hydrophobic Oil (Core) | Soybean Oil, USP (super refined) |
| Organic Solvent | Dehydrated Alcohol, USP (anhydrous |
| | ethanol) |
| Surfactant | Polysorbate 80, NF |
| Emulsifying Agent | Cetylpyridinium Chloride, USP |
| Preservative | |

(2) Recombinant F-protein: (baculovirus host—Sino Biological Inc. Cat 11049-V08B); (3) Phosphate Buffered Saline (sterile) 1X: Supplied by CellGro; (4) Test animal: BALB/c mice 8-10 weeks old, females (The Jackson Laboratory).

Review of study design: Three groups of BALB/c mice were immunized against F-protein as follows: (1) Prime immunization: Group I—4.45 μg F-protein+20% W₈₀5EC at the total volume 15 μl (n=8); Group II—4.45 μg F-protein at the total volume 15 μl (n=5); and Group III—PBS at the total volume 15 μl (n=10); and (2) Boost immunization: Group I—10 μg F-protein+20% W₈₀5EC at the total volume 15 μl (n=8); Group II—10 μg F-protein at the total volume 15 μl (n=5); and Group III—PBS at the total volume 15 μl (n=10).

Animals were divided randomly into three groups. Groups were immunized on day 0 intranasally (into nares, half volume per nare). Animals were bled every 2 weeks for the duration of the experiment. The mice were intranasally inoculated with 10⁵ PFU L19 RSV 14 days following the final boost.

Methods: Test formulation: The vaccine mixture was formulated as follows. First immunization: (1) 90 μl of recombinant F protein (conc. 0.445 mg/ml) was mixed with 45 μl of 60% W₈₀5EC. Final concentrations: F protein—0.3mg/ml; NE—20%. Volume dose—15 μl/animal. (2) 50 ul of recombinant F protein (conc. 0.445 mg/ml) was mixed with 25 μl of PBS 1X. Final concentrations: F protein—0.3mg/ml; NE—0%. Volume dose—15 μl/animal. For the immunization boost: (1) 90 μl of recombinant F protein (conc. 1 mg/ml) was mixed with 45 μl of 60% W₈₀5EC. Final concentrations: F protein—0.67 mg/ml; NE—15%. Volume dose—15 μl/animal; and (2) 50 μl of recombinant F protein (conc. 1 mg/ml) was mixed with 25 μl of PBS 1X. Final concentrations: F protein—0.67mg/ml; NE—0%. Volume dose—15 μl/animal.

Test methods. Vaccination procedure: Mice were anesthetized with isoflurane and positioned with their heads reclined about 45° then 7.5 μl vaccine was administered into the left nare. The animals were re-anesthetized and restrained as above. The remaining 7.5 μl of the vaccine was administered into the right nare. Physical examination: Body posture, activity, and pilorection were monitored on weekly basis for each individual animal in the study. Bleeding: Two, 4 and 6 weeks after the first immunization mice were bled by saphenous phlebotomy.

Serum ELISA: Antigen-specific IgG, IgG1, IgG2a, IgG2b, and IgE responses were measured by ELISA with 5 μg/ml of F-protein for plate coating. Anti-mouse IgG -alkaline phosphatase conjugated antibodies were from Jackson ImmunoResearch Laboratories Inc. (West Grove, Pa.). Alkaline phosphatase (AP) conjugated rabbit anti-mouse IgG (H&L), IgG1, IgG2a, IgG2b, IgG2c and IgE were purchased from Rockland Immunochemical s, Inc. (Gilbertsville, Pa.).

Intranasal challenge with live L19 RSV: Mice were challenged with live, infectious RSV intranasally (10⁵ PFU) 2 weeks post boost immunization.

Airway hyperreactivity (AHR): AHR was measured using a Buxco mouse plethysmograph which is specifically designed for the low tidal volumes (Buxco). The mouse to be tested was anesthetized with sodium pentobarbital and intubated via cannulation of the trachea with an 18-gauge metal tube. The mouse was subsequently ventilated with a Harvard pump ventilator (tidal volume=0.4 ml, frequency=120 breaths/min, positive end-expiratory pressure 2.5-3.0 cm H2O). The plethysmograph was sealed and readings monitored by computer. As the box is a closed system, a change in lung volume will be represented by a change in box pressure (Pbox) that was measured by a differential transducer. Once baseline levels had stabilized and initial readings were taken, a methacholine challenge was given via tail vein injection. After determining a dose-response curve (0.01-0.5 mg), an optimal dose was chosen, 0.250 mg of methacholine. This dose was used throughout the rest of the experiments in this study. After the methacholine challenge, the response was monitored and the peak airway resistance was recorded as a measure of airway hyperreactivity.

Euthanasia and biological material harvest procedure: The mice were euthanized by isoflurane asphyxiation. Lung-associated lymph nodes were harvested for immune response evaluation. Intranasal inoculation of mice with Line 19 RSV, leads to an infection that is associated with a moderate form of disease phenotype, including mucus hypersecretion and inflammation. The severity of this phenotype in control and immunized animals was assessed using histologic analysis and QPCR for viral and cytokine gene expression as well as mucus-associated genes Muc5ac and Gob5.

Quantitative PCR: The smallest lung lobe was removed and homogenized in 1 ml of Trizol reagent (Invitrogen). RNA was isolated as per manufacturer's protocol, and 5 μg was reverse-transcribed to assess gene expression. Detection of cytokine mRNA in lung samples was determined using pre-developed primer/probe sets (Applied Biosystems) and analyzed using an ABI Prism 7500 Sequence Detection System (Applied Biosystems). Transcript levels of Muc5ac, Gob5 were determined using custom primers, as previously described. Gapdh was analyzed as an internal control and gene expression was normalized to Gapdh. Fold changes in gene expression levels were calculated by comparison to the gene expression in uninfected mice, which were assigned an arbitrary value of 1. RSV transcripts were amplified using SYBR green chemistry, by adapting previously published primer sets to match the sequence of Line 19.

Plaque assays: Lungs of mice were excised, weighed, and homogenized in 1×EMEM (Lonza). Homogenates were clarified by centrifugation (5000×g for 10 mins), serial dilutions were made of the supernatant and added to subconfluent Vero cells. After allowing the virus to adhere for one hour, the supernatant was removed, and replaced with 0.9% methylcellulose/EMEM. Plaques were visualized on day 5 of culture by immunohistochemical techniques using goat anti-RSV as the primary antibody (Millipore), HRP-rabbit anti-goat antibody as the secondary, and 4-chloronapthol (Pierce) as the substrate.

Lymph node restimulation: Lung associated lymph node (LALN) cell suspensions were plated in duplicate at 1×10⁶ cells per well followed by restimulation with either media or RSV (MOI˜0.5). Cells were incubated at 37° C. for 24 hours and supernatants collected for analysis on the BioRad Bioplex 200 system according to the manufacturer's protocol. Kits (BioRad) containing antibody beads to Th cytokines (IL-17,

IFNγ, IL-4, IL-5, IL-13) were used to assay for cytokine production in each of the samples.

Histology: Right lobes of the lungs were isolated and immediately fixed in 10% neutral buffered formalin. Lung samples were subsequently processed, embedded in paraffin, sectioned, and placed on L-lysine-coated slides, and stained using standard histological techniques using Hemotoxylin and Eosin (H&E) and Periodic-acid Schiff (PAS). PAS staining was done to identify mucus and mucus-producing cells.

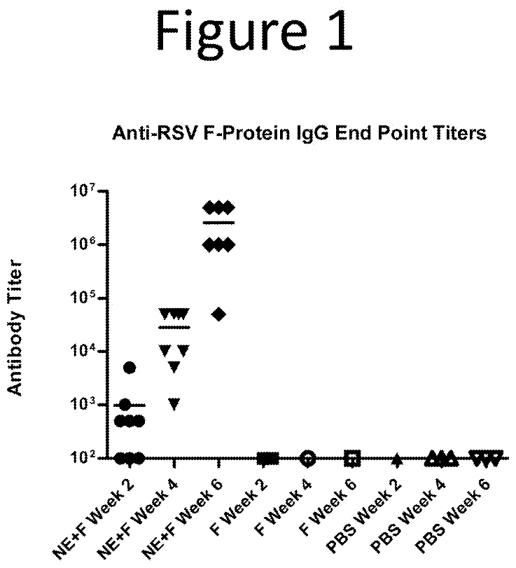

Results. Evaluation of humoral response. Evaluation of specific serum IgG. Sera obtained from mice 2, 4 and 6 weeks after the prime immunization were used to assess the endpoint titer of specific IgG using ELISA. Endpoint titer was defined as the highest sera dilution yielding absorbance three times above the background. Endpoint titer results are shown in FIG. 1. Only nanoemulsoin F-protein immunized mice responded vaccination by high titers of specific anti-F-protein IgG antibodies with group average titers approaching 5×10⁶ at week 6.

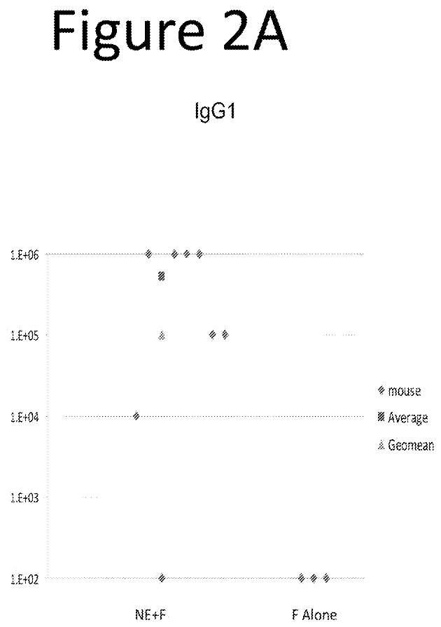

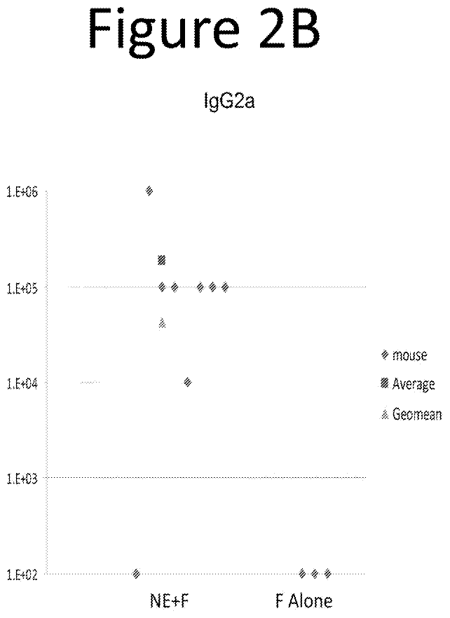

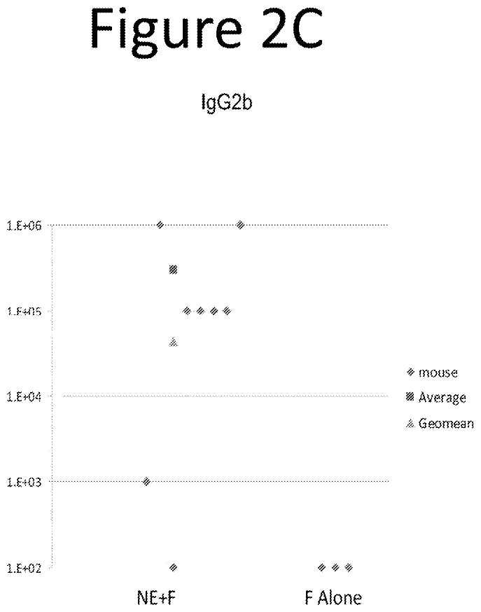

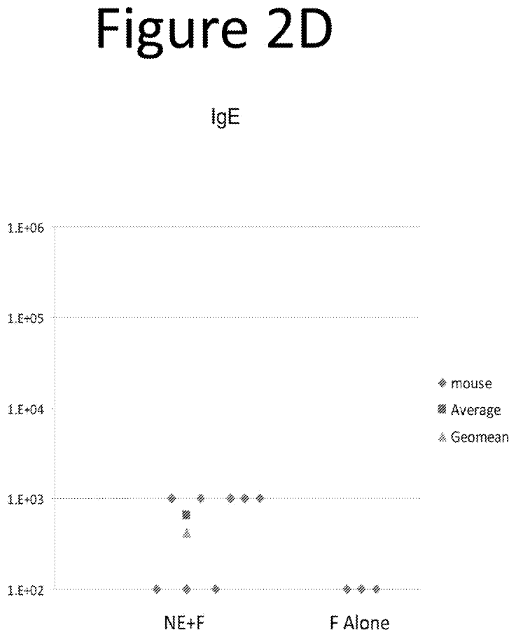

Evaluation of specific IgG1, IgG2a, IgG2b, and IgE humoral response in sera to immunization. Sera obtained from mice two weeks after the second immunization (week 6) were used to assess the endpoint titer of specific IgG1, IgG2a, IgG2b, and IgE using ELISA (FIG. 2). Endpoint titer was defined as the highest serum dilution yielding absorbance three times above the background. NE+F-protein immunized mice produced high levels of levels of specific IgG1, IgG2a, IgG2b antibodies (FIGS. 2A, 2B and 2C). Serum IgE titers were low but present and averaged around 663 for NE+F-protein immunized mice (FIG. 2D).

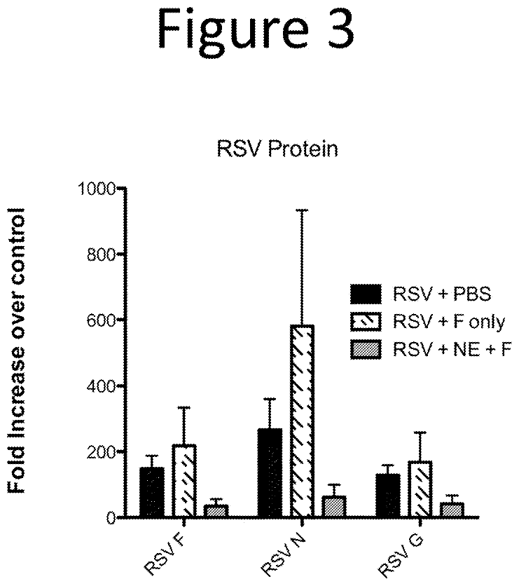

RSV Challenge: RSV Gene expression in lungs 8 days following challenge. A challenge study was conducted to determine whether vaccination with NE-F-protein would protect the mice from respiratory challenge with RSV. At 6 weeks following prime immunization, mice were challenged with live, infectious RSV intranasally (10⁵ PFU). On day 8 post-challenge, viral load was assessed in the lungs via QPCR and via plaque assay. As assessed via QPCR, a significant decrease in the transcript levels for RSV F and RSV N and RSV G were detected in the lungs of NE-F-protein vaccinated mice in comparison to non-immunized and F-protein only immunized mice (FIG. 3). These data indicate that NE-F-protein vaccine dramatically improves viral clearance in the following lower respiratory challenge.

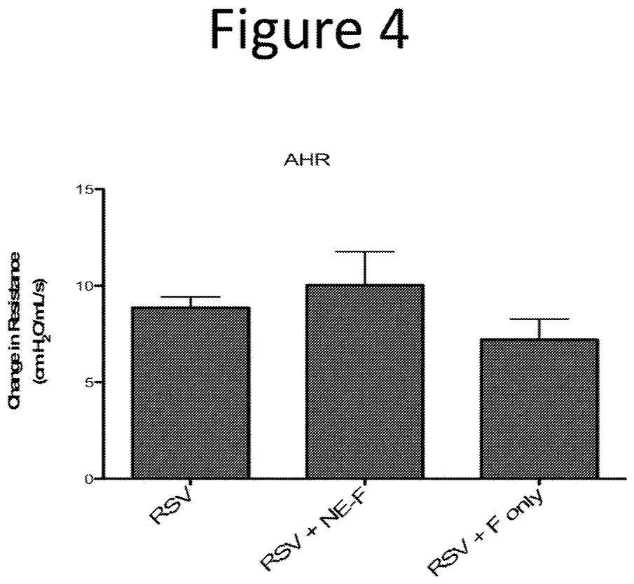

Nanoemulsion+ —RSV does not promote airway hyperreactivity. As previously reported, vaccination with formalin fixed RSV promotes the development of airway hyperreactivity (AHR) and eosinophilia upon live viral challenge. With this in mind, whether nanoemulsion+F-protein vaccination promotes airway hyper-reactivity, or other evidence of immunopotentiation, was evaluated. Compared to control RSV infected mice, nanoemulsion-RSV immunized mice exhibited only baseline increases in airway resistance following intravenous methacholine challenge (FIG. 4).

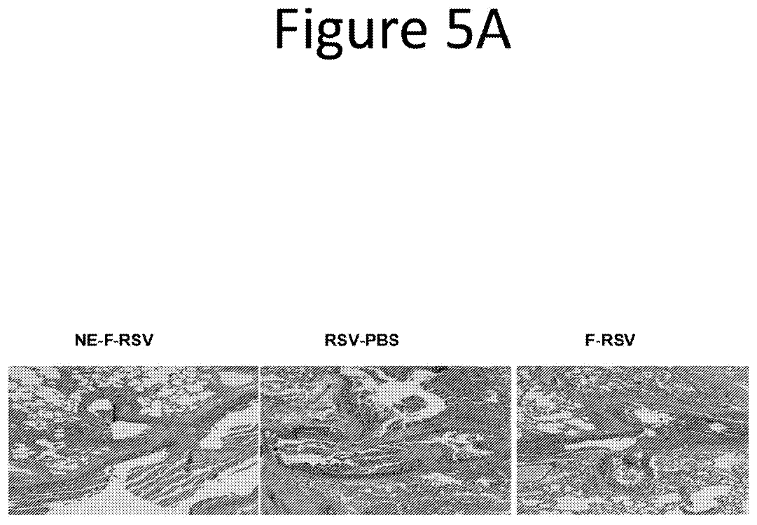

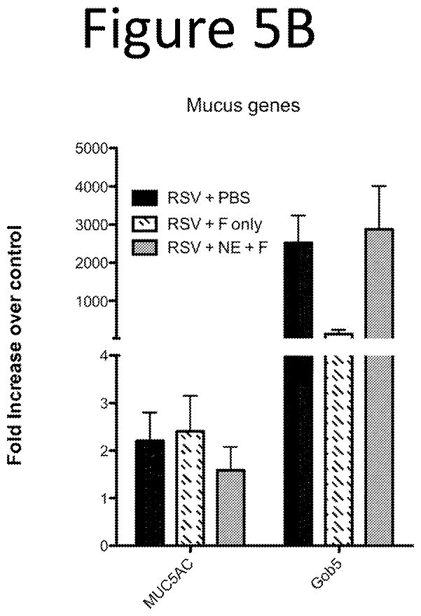

Nanoemulsion+ F-protein immunization is associated with mucus secretion following live challenge. Intranasal inoculation of mice with Line 19 RSV, leads to an infection that is associated with a moderate form of disease phenotype, including mucus hypersecretion and inflammation. The severity of this phenotype in control and immunized animals was assessed using histologic analysis and QPCR for viral and cytokine gene expression. At day 8, post-challenge, NE+F-protein vaccinated mice exhibited similar mucus hypersecretion compared to challenged non-immunized mice, as assessed via histologic analysis (FIG. 5A). Similar expression of the mucus-associated genes Muc5ac and Gob5 was measured in NE-F-protein immunized mice compared to non-immunized controls (FIG. 5B).

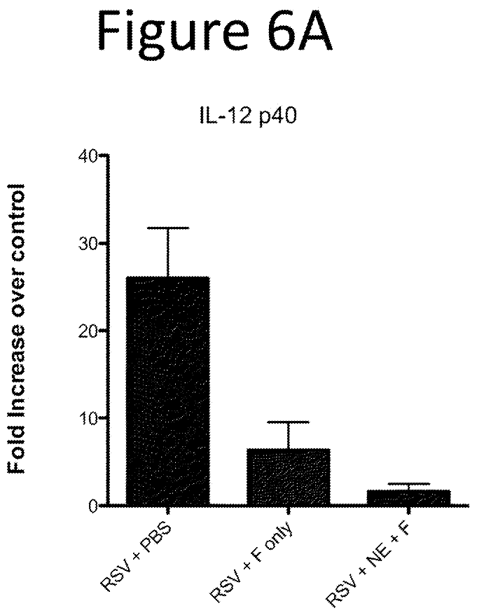

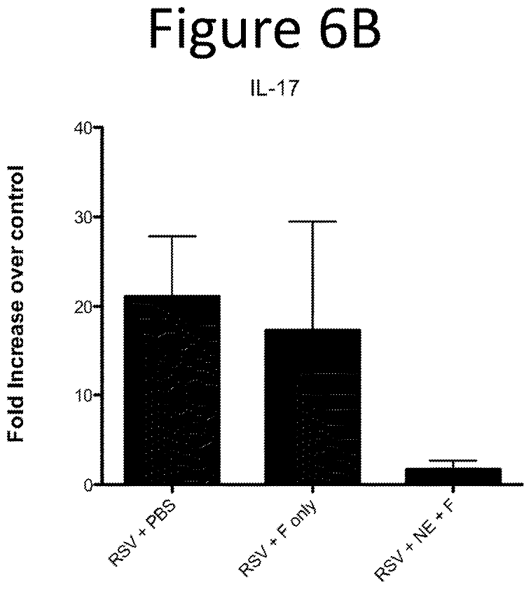

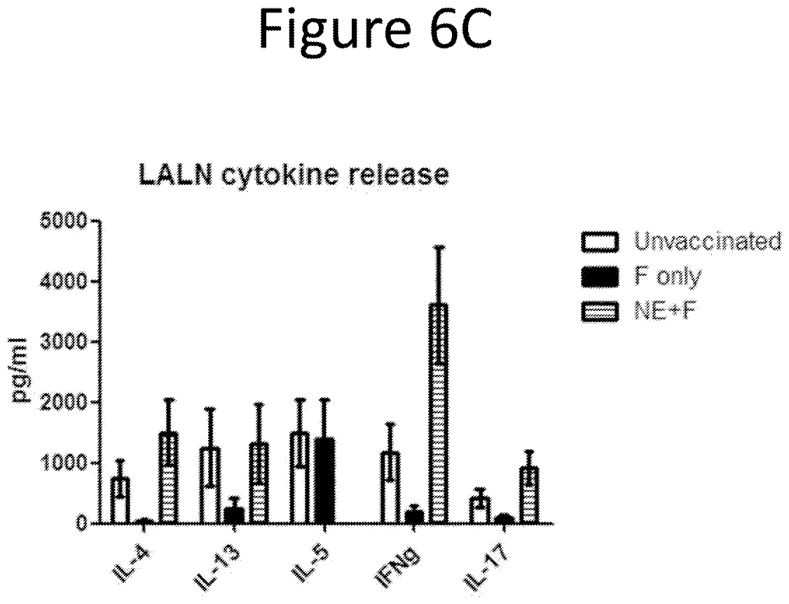

Nanoemulsion+ F-protein immunization promotes induction of mixed Thl and Th2 cytokines following challenge. The further characterize the immunization phenotype that promoted viral clearance in nanoemulsion+F-protein immunized; we used QPCR for cytokine gene expression. Compared to control mice, nanoemulsion+F-protein vaccinated mice did not exhibit IL-12 and IL-17, as assessed by the levels of RSV transcripts in the lungs at day 8 post challenge (FIGS. 6A and B respectively). As a means of validation, cytokine profiles were assessed in bronchoalveolar lavage and lung homogenates via multiplex antibody-based assay (Bioplex). NE-RSV vaccination showed an enhanced IFN-y response. IL-17 showed increase production compared to unvaccinated mice (FIG. 6C).

Conclusions: Only the group immunized with 20% Wz₈₀5EC mixed with F-protein responded to vaccination with high titers of specific anti-RSV IgG, IgG1, IgG2a and IgG2b antibodies. This was associated with minimal production of IgE. Nanoemulsion+F-protein vaccination was also associated with enhanced viral clearance and protection following live RSV challenge. Interestingly, the phenotype of the immune response was not associated with production of IL-12 or IL-17.

A mixed Th1, Th2 pattern of cytokine release was observed for NE+F-protein immunized mice both in lymph nodes after re-stimulation in vitro with RSV L19. However, this was not associated with immunopotentiation although significant mucus production was observed.

### Example 6

### Testing Whole Virus RSV Vaccines for Immune Response in Cotton Rats

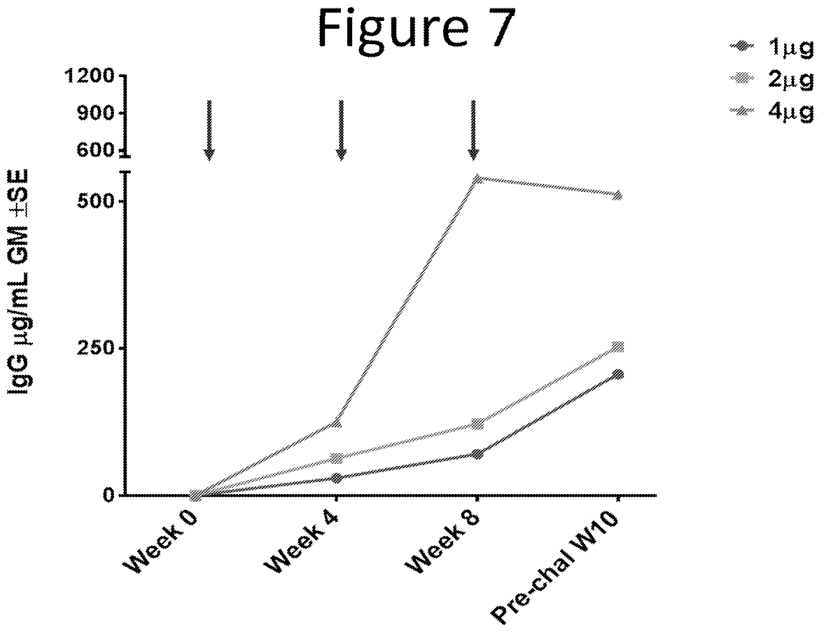

The purpose of this example was to determine the neutralizing activity of antibodies produced in cotton rats vaccinated with RSV vaccines. The data derived from these experiments is shown in FIGS. 7-12.

Briefly, cotton rats were anesthetized with isoflurane and immunized intramuscularly (IM, quadriceps) with 0.1 ml vaccine. Nanoemulsion 01 (NE01) (5%W₈₀5EC/RSV L19), or Nanoemulsion 01 (NE03) (20% DODAC/5%CPC/RSV L19) vaccines. Several controls were used for comparison: (i) RSV infection of naïve, mock-immunized animals (negative control for efficacy), (ii) infection and re-infection with RSV (positive control for efficacy), and (iii) RSV infection of FI-RSV-immunized animals (control for vaccine-enhanced disease). FI-RSV Lot#100 was produced in the mid-1960s by Pfizer Inc. (NIH contract PH43-63-582) and stored at 4° C., diluted 1:100 using PBS, and injected within an hour of preparation.

Emulsions

Emulsions were formed by emulsification of an oil, purified water, nonionic detergent, organic solvent and surfactant, such as a cationic surfactant and/or cationic lipid (see, e.g., Example 3). All components of emulsion compositions of the invention were included on the FDA list of approved inactive ingredients for Approved Drug Products. An exemplary specific nanoemulsion adjuvant was designated as “60%W₈₀5EC” or 60% DODAC/CPC NE″. The 60%W₈₀5EC-vaccine adjuvant or 60% DODAC/CPC-vaccine adjuvant was composed of the ingredients shown in Table 11 below: purified water (USP), soybean oil (USP), dehydrated alcohol (anhydrous ethanol) (USP), polysorbate 80 (NF), cetylpyridinium chloride (CPC) (USP), and/or dioctadecyldimethylammonium chloride (DODAC). All components of this exemplary nanoemulsion adjuvant were included on the FDA list of approved inactive ingredients for Approved Drug Products.

TABLE 11
Composition of Nanoemulsion (NE) Adjuvants (w/w %)

| | 60% | 60% |
|---|---|---|
| Ingredients | W₈₀5EC^{a} | DODAC/CPC^{b} |
| Purified Water, USP | 54.10% | 54.09% |
| Soybean Oil, USP | 37.67% | 37.67% |
| Dehydrated Alcohol, USP | 4.04% | 4.04% |
| (anhydrous ethanol) | | |
| Polysorbate 80, NF | 3.55% | 3.40% |
| Cetylpyridinium Chloride (CPC), | 0.64% | 0.16% |
| USP | | |
| Dioctadecyldimethylammonium | — | 0.64% |
| chloride (DODAC) | | |

^{a}Used to prepare NE01;
^{b}Used to prepare NE03

Vaccine preparation of NE01 (5%W₈₀5EC/RSV L19) and NE03 (20% DODAC/5%9CPC/RSV L19) vaccines compositions: Vaccines were prepared by mixing recombinant respiratory syncytial virus (RSV) F protein with specific 60% nanoemulsion to the final desired protein concentration of (e.g. X μg/dose) at the 5% or 20% final nanoemulsion concentration (e.g. NE01: 5% W₈₀5W₈₀5EC/RSV L19 vaccine or NE03: 20% DODAC/5%CPC/RSV L19 vaccine).

Dual chain lipids/surfactants include: The type of cationic lipid utilized in an emulsion and/or composition (e.g., immunogenic composition (e.g. vaccine)) formulated for nasal and/or injectable administration is not particularly limited. Multiple lipophilic side chain amphiphilic substances that have two or more lipophilic side chains (e.g., attached to a polar head group) may be used. Such lipids may be nonionic, cationic, anionic, or zwitterionic in nature.

Several cationic dual chain lipids, such as TAP and DAP, may possess a variety of types of chain groups having carbon atom to number of saturated bonds ratios of, for example, 14:0, 16:0, 18:0, and 18:1, as well as a variety of types of acyl groups having from about 10 carbon atoms to about 18 carbon atoms such as dimyristoyl, dipalmitoyl, distearoyl, and dioleoyl. The invention is not limited by the amount of dual chain lipids/surfactant in an emulsion and may range, based upon the total weight of the composition, from about 0.1% to about 95% (such as from about 10% to about 65%).

Optimized Dosing

First, the optimized dosing schedule was determined. Cotton rats were given intramuscular (IM) injections of nanoemulsion (NE) RSV vaccine comprising whole virus RSV-L19 at doses corresponding to 1, 2, and 4 μg of F protein. Three injections were given, each 4 weeks apart, as shown in FIG. 7. IgG response was optimized following two vaccinations at the 4 μg dose.

Challenge Study

Next, a challenge study was performed in cotton rats. Rats were subdivided into five treatment groups, each with N=5. The treatment groups included: (1) IM NE01-RSV L19 (5%W₈₀5EC/RSV L19) vaccine (6 μg F protein), (2) IM NE03-RSV L19 (20% DODAC/5%CPC/RSV L19) vaccine (6 μg F protein), (3) IM Alum Fl-RSV vaccine-control for vaccine-enhanced disease (FI-RSV Lot#100 was produced in the mid-1960s by Pfizer Inc. (NIH contract PH43-63-582) and stored at 4° C., diluted 1:100 using PBS, and 0.1 ml intramuscularly injected within an hour of preparation), (4) RSV A2 infection (10⁵ pfu/dose) or (5) PBS control. Cotton rats were given an initial vaccination/injection on day 0 and a second vaccination/injection on day 28, RSV A2 infection (group 4) was given on day 0

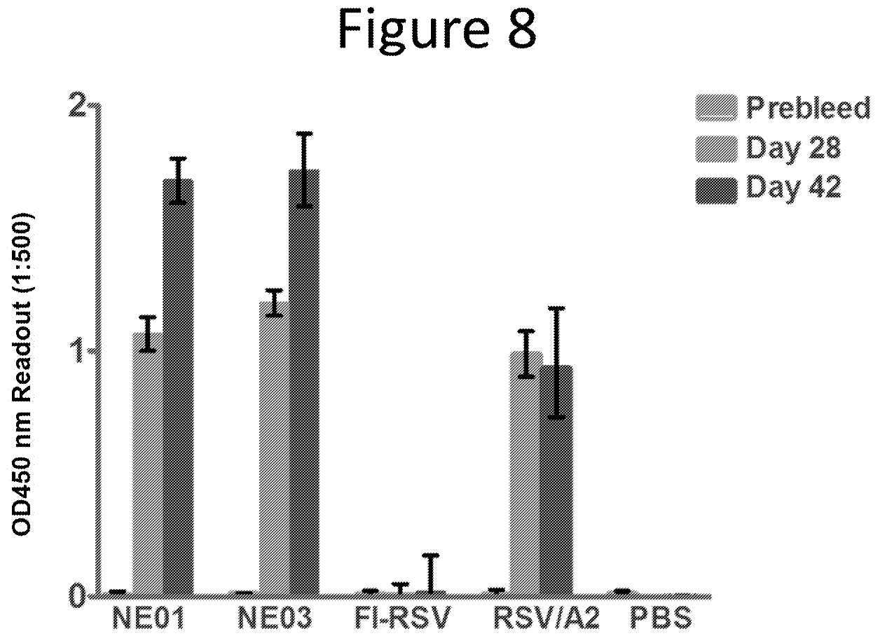

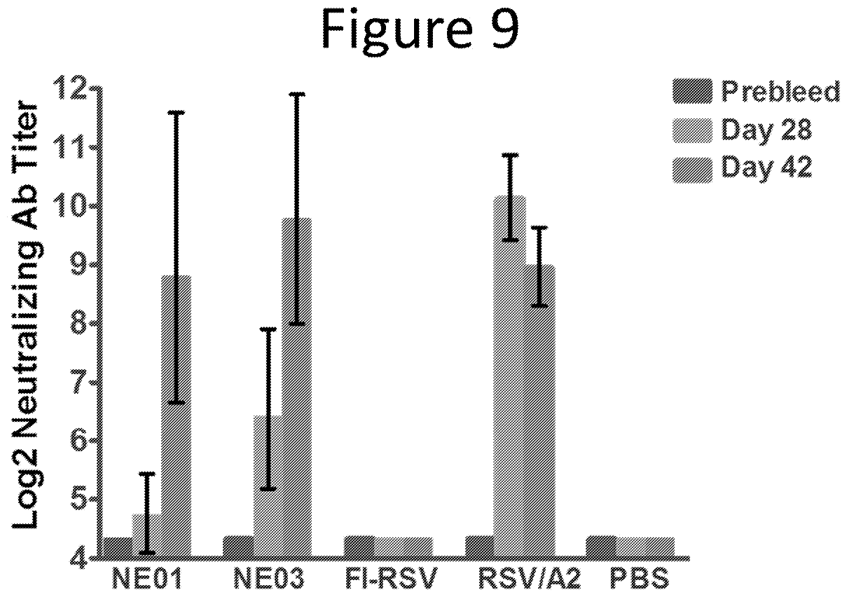

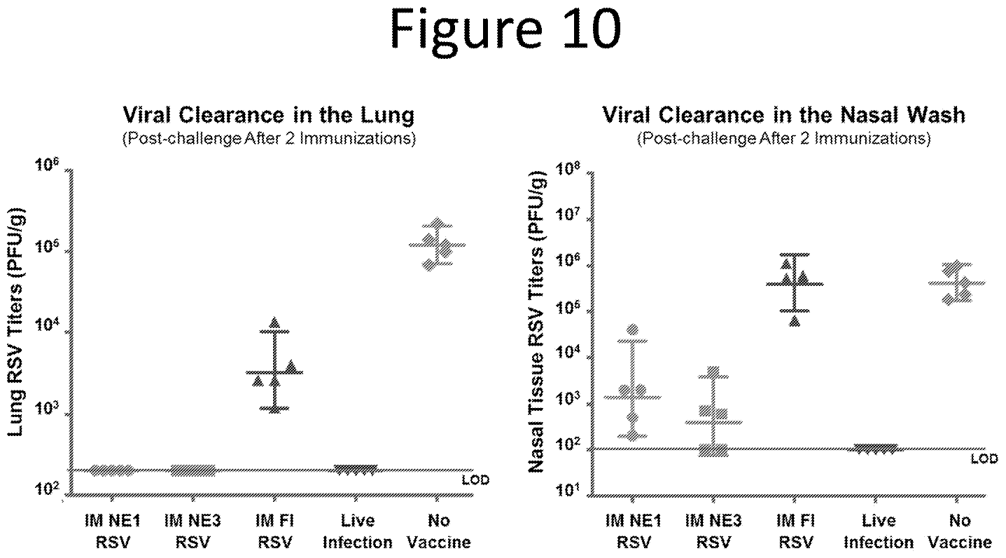

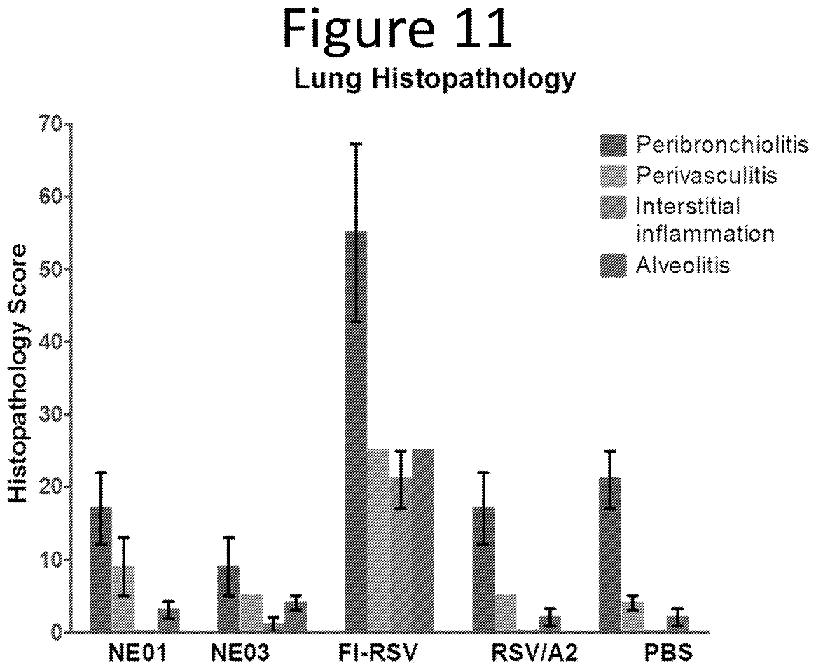

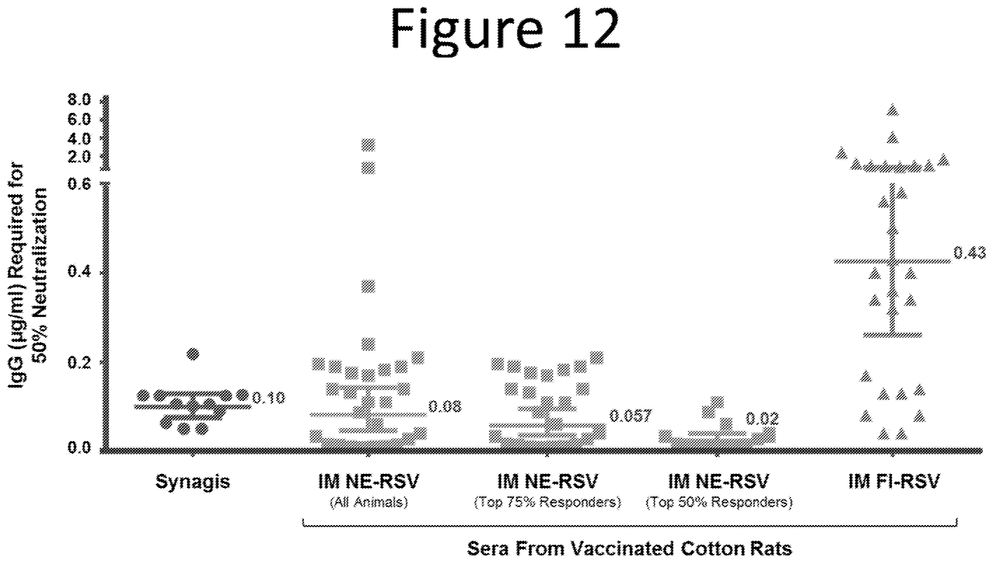

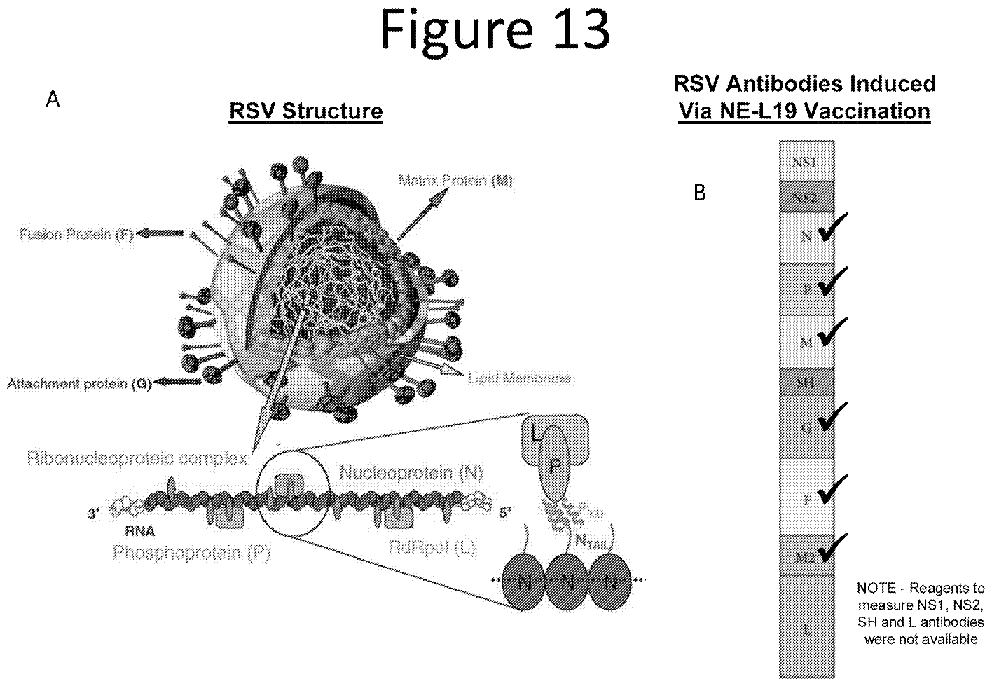

42 days after the initial vaccination injection, rats were challenged with 10⁵ PFU of RSV A2. Following the RSV challenge, rats were analyzed to determine serum IgG (FIG. 8), neutralizing antibody titers (FIG. 9), viral clearance in the lung and nasal cavity (FIG. 10), lung histopathology (FIG. 11), and neutralizing activity of serum compared to Synagis®, the only currently clinically approved RSV treatment (FIG. 12).

Physical examination: Body posture, activity, and pilorection were monitored on weekly basis for each individual animal in the study.

Bleeding: Before, 4 and 8 weeks after the first immunization cotton rats were eyebled.

Lung and Nose Viral Titration

Lung and nose homogenates were clarified by centrifugation and diluted in EMEM. Confluent HEp-2 monolayers were infected in duplicates with diluted homogenates in 24 well plates. After one hour incubation at 37° C. in a 5% CO2 incubator, the wells were overlayed with 0.75% methylcellulose medium. After 4 days of incubation, the overlay was removed and the cells were fixed with 0.1% crystal violet stainfor one hour and then rinsed and air dried. Plaques are counted and virus titer was expressed as plaque forming units per gram of tissue. Viral titers were calculated as geometric mean ± standard error for all animals in a group at a given time.

Pulmonary histopathology

Lungs were dissected and inflated with 10% neutral buffered formalin to their normal volume, and then immersed in the same fixative solution. Following fixation, the lungs were embedded in paraffin, sectioned and stained with hematoxylin and eosin (H&E). Four parameters of pulmonary inflammation were evaluated: peribronchiolitis (inflammatory cell infiltration around the bronchioles), perivasculitis (inflammatory cell infiltration around the small blood vessels), interstitial pneumonia (inflammatory cell infiltration and thickening of alveolar walls), and alveolitis (cells within the alveolar spaces). Slides were scored blind on a 0-4 severity scale. The scores were subsequently converted to a 0 -100% histopathology scale.

Real-time PCR

Total RNA was extracted from homogenized tissue using the RNeasy purification kit (QIAGEN). One μg of total RNA was used to prepare cDNA using Super Script II RT (Invitrogen) and oligo dT primer (1 μl , Invitrogen). For the real-time PCR reactions the Bio-Rad iQTM SYBR Green Supermix was used in a final volume of 25 μl, with final primer concentrations of 0.5 μM. Reactions were set up in duplicates in 96-well trays. Amplifications were performed on a Bio-Rad iCycler for 1 cycle of 95° C. for 3 min, followed by 40 cycles of 95° C. for 10 seconds (s), 60° C. for 10 s, and 72° C. for 15 s. The baseline cycles and cycle threshold (Ct) were calculated by the iQ5 software in the PCR Base Line Subtracted Curve Fit mode. Relative quantitation of DNA was applied to all samples. The standard curves were developed using serially diluted cDNA sample most enriched in the transcript of interest (e.g., lungs from 6 hours post RSV infection of FI-RSV-immunized animals). The Ct values were plotted against log10 cDNA dilution factor.

These curves were used to convert the Ct values obtained for different samples to relative expression units.These relative expression units were then normalized to the level of (3-actin mRNA (“housekeeping gene”) expressed in the corresponding sample. For animal studies, mRNA levels were expressed as the geometric mean ± SEM for all animals in a group at a given time.

RSV Neutralizing Antibody Assay (60% PRNT)

Heat inactivated sera samples were diluted 1:10 with EMEM and serially diluted further 1:4. Diluted serum samples were incubated with RSV/A2 (25-50 PFU) for 1 hour at room temperature and inoculated in duplicates onto confluent HEp-2 monolayers in 24 well plates. After one hour incubation at 37° C. in a 5% CO2 incubator, the wells were overlayed with 0.75% methylcellulose medium. After 4 days of incubation, the overlays were removed and the cells were fixed and stained with 0.1% crystal violet for one hour and then rinsed and air dried. The corresponding reciprocal neutralizing antibody titers were determined at the 60% reduction end-point of the virus control using the statistics program “plqrd.manual.entry”. The geometric means ±standard error for all animals in a group at a given time were calculated.

RSV Binding IgG Antibodies (ELISA)

Purified F protein extracted from RSV/A2-infected HEp-2 cells was used as a coating antigen in a 96 well ELISA plate format. The plates were incubated in blocking solution for one hour at room temperature and subsequently washed. Serially diluted sera (1:500, 1:2,000, 1:8,000, and 1:32,000 in duplicates) along with the positive and negative controls were added to the wells and incubated at room temperature for one hour. After washing the plates, Rabbit anti Cotton Rat IgG (1:4,000) was added to all the wells and incubated for one hour at room temperature. After additional washes, the plates were incubated with Goat anti Rabbit IgG-HRP (1:4,000) for one hour at room temperature. For development of the assay, TMB substrate was added to all the wells and incubated at room temperature for 15 minutes. TMB-Stop solution was added to all the wells and optical density at 450 nm (0D450) is recorded. Geometric mean of the optical density was measured for all duplicate sera samples.

### Example 7

### Specificity of RSV L19 Nanoemulsion Vaccine-Induced Rresponse

The purpose of this experiment was to determine if animals immunized with nanoemulsion (NE)-adjuvanted RSV L19 vaccine produce antibodies that can recognize multiple antigens of both L19 and A2 strains of RSV, as well as recombinant F and G proteins, two major protective antigens of the virus.

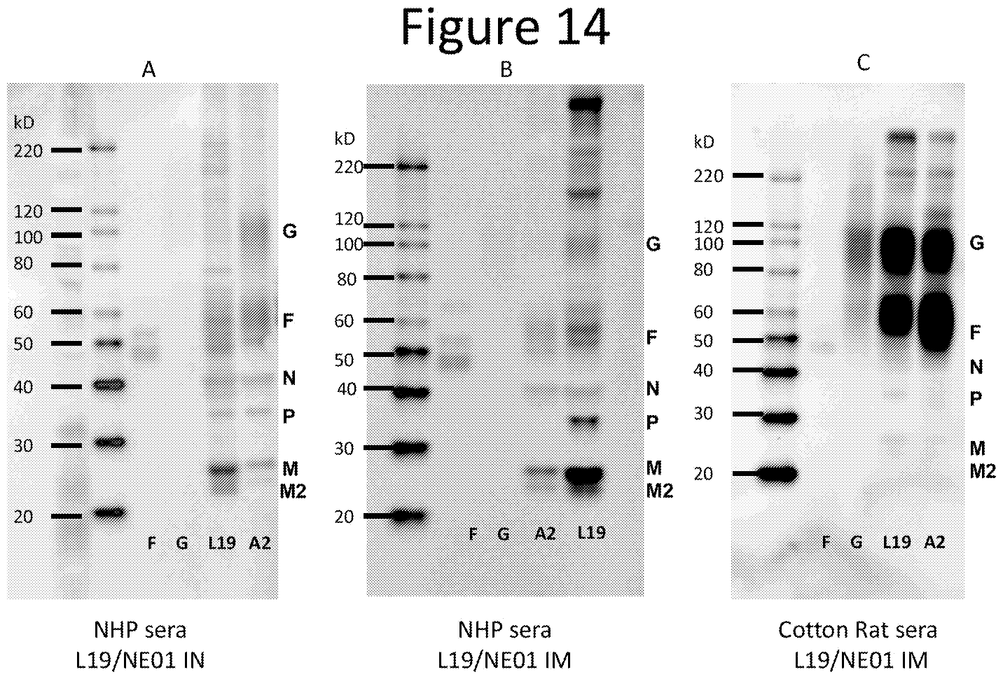

In this experiment, the reactivity of sera obtained from animals immunized with NE adjuvanted whole RSV L19 vaccine was assessed using Western Blot assays. Animals were immunized via intranasal (IN) (FIG. 14A) and intramuscular (IM) (FIGS. 14B and C) routes. The reactivity of non-human primate (NHP) and cotton rat (CR) sera was determined against RSV L19 and A2 strains, and recombinant F (rF) and G (rG) proteins (FIGS. 14A, B, and C).

The results indicate that both IN and IM immunizations elicit antibodies with a different pattern of recognition of the recombinant and natural viral proteins. However, vaccination with NE01/RSV L19 vaccine either IN or IM, both non-human primates (NHP) and cotton rats (CR), produced antibodies that showed reactivity against L19 and cross reactivity against A2 virus.

Procedures, Materials and Reagents

Three studies were conducted: (1) five (5) non-human primates immunized intranasally with 20% W₈₀5EC combined with 50 μg of F protein (L19 RSV virus based on F protein content) (FIG. 14A); (2) five (5) non-human primates immunized intramuscularly with 5% W₈₀5EC combined with 20 μg of F protein (L19 RSV virus based on F protein content) (FIG. 14B); and (3) four (4) different cotton rats immunized IM with 5% W805EC combined with 4, 8, or 15 μg of F protein (L19 RSV virus based on F protein content) (FIG. 14C). In both the NHP and CR studies, the animals were vaccinated three times at days 0, 28, and 56. Sera from each of the studies was pooled and evaluated for immunogenicity against various RSV proteins (FIGS. 14A, B, and C).

Reagents are shown in Table 12.

TABLE 12

| Item | Company |
|---|---|
| Criterion XT Cell | Biorad |
| Model 1000/500 Power Supply | Biorad |
| 4-12% Criterion XT Precast Gel (26 well, 15 ul) | Biorad |
| XT MES Running Buffer (20x) | Biorad |
| Precision Plus Prestained Ladder | Biorad |
| MagicMark ™ XP Western Protein Standard | Invitrogen |
| 10x Tris/Glycine Transfer Buffer | Biorad |
| 4x Laemmli Buffer | Biorad |
| Supersignal West Pico Plus | ThermoFisher |
| Dithiothreitol | Biorad |
| Criterion Blotter Filter Paper | Biorad |
| Methanol | J. T. Baker |
| Nonfat Dry Milk Powder | Carnation |
| Immobilon-P Transfer Membrane (PVDF), 0.45 μm | Millipore |
| Purified (Reverse osmosis) or distilled water | NanoBio |
| Non-Human Primate IN anti-L19 polyclonal antibody | NanoBio |
| Non-Human Primate IM anti-L19 polyclonal antibody | NanoBio |
| Cotton Rat IM anti-L19 polyclonal antibody sera | NanoBio |
| Goat Anti-Monkey IgG | Rockland |
| Chicken anti-Cotton Rat IgG (H + L) | Immunology |
| | Consultants |
| Recombinant Human RSV Fusion glycoprotein F0 | Sino Biological |
| | Inc |
| Recombinant Human RSV Fusion glycoprotein G | Sino Biological |
| | Inc |
| RSV A2 | NanoBio |
| RSV L19 | NanoBio |
| 10x TBS, pH 7.5 | Corning |

The goal of the Western blot analysis was to show the variety of antibodies generated following vaccination with NE RSV L19. To summarize, recombinant F and G proteins were loaded on the gel at 200 ng/well. RSV A2 and RSV L19 virus was prepared and then loaded on the gel at 200 ng/well based on F protein content. The gels were blotted onto PVDF membranes and blocked with 5% non-fat dehydrated milk in TTBS. The blots were probed with pooled serum from either non-human primates (NHP) or cotton rats (CR) immunized with NE L19 by IN or IM immunization.

The results showed IN (FIG. 14A) or IM (FIG. 14B) immunization with NE L19 in NHP's induced antibodies against several RSV viral proteins: F, G, P, N, M and M2 of homologous L19 and heterologous A2 strains of RSV along with recognizing the recombinant F protein. CR sera (FIG. 14C) showed stronger reactivity against natural F and G and recombinant G as compared with NHP. While the pattern of recognition was different for CR and NHP, the ability of the sera to recognize several viral antigens, as well as two major protective viral antigens indicates that the vaccine will offer protection against both viral strains.

Preparation of Reagents: (1) Sample preparation: samples were prepared by first adding reducing sample buffer to samples to obtain a final concentration of 1×. Samples were then heated at 100° C. for 5minutes and spun down at 1,000 g for 10 seconds prior to sample loading; (2) 1× Running Buffer: 20× XT MES SDS Running Buffer in water; (3) 1× TBST: 1× TBS buffer with 0.1% Tween-20; (4) 1× Transfer Buffer: 10× Tris/Glycine transfer buffer, 10% methanol in water; (5) Primary antibody: 1:750 dilution of NHP anti-L19 IN and IM, 1: 1,000 dilutions of CR Anti-L19 in 5% NFDM, 1× TBST; (6) secondary antibody: (a) 1:10,000 anti-monkey antibody in 5% NFDM-1× TTBS; and (b) 1:10,000 anti-cotton rat antibody in 5% NFDM-1× TTBS; and (7) 7. 5% NFDM in 1× TTBS (1× Blocking buffer): 5 gram/100 mL non-fat dry milk in 1 × TBS-T.

Procedures: Samples were prepared as described above and SDS PAGE was conducted as per conditions shown in Table 13.

TABLE 13
SDS-PAGE

| | | Incubation Time and |
|---|---|---|
| | Reagent | Conditions |
| Sample | Mix sample in 1x reducing | 5 minutes at 100° C. |
| Preparation | sample buffer | |
| Sample | Equally load samples based | NA |
| Loading | on protein concentrations | |
| Gel Running | 100 volts for 2 hours | 2 hour at room |
| | | temperature |

Criterion XT gels were inserted into running tank and 1x running buffer was added. Samples were loaded based on equal amount of protein in all lanes. Recombinant F and G proteins, along with RSV Virus A2 and L19 were loaded in equal amounts based on their F protein concentrations.

SDS-PAGE Gel Electrophoresis was conducted as following conditions: (a) 100V for 120 minutes or until the dye front is at the bottom of the gel; (b) after the completion of run, gel cassettes were removed from the tank and gels were removed by opening the cassettes; (c) Western Blot transfer was performed immediately.

Western Blot analysis was conducted following conditions prescribed in Table 14: (a) proteins were transferred onto PVDF membrane in ice cold lx transfer buffer at 100 volts for 1 hour; (b) membrane was blocked with 5% Non-Fat Dry Milk in 1xTBST at RT for at least 1 hour with rocking; (c) primary antibody incubation (i) appropriate antibody diluted in 5% milk in TBST was added and incubated at RT for at least 2 hours, or at 4° C. overnight with rocking; (d) blot was washed 3×5 minutes each in TBST; (e) secondary antibody incubation (i) appropriate secondary antibody diluted in 5% milk with TBST was added and incubated at RT for 1-2 hours with rocking; (f) blot was washed 3 x 5 minutes each in TBST; (g) signal detection (i) SuperSignal West Pico substrate was applied to the blot and incubated at RT for 5 minutes or until desired color was developed; and (h) Scan the membrane (i) blot was scanned immediately after substrate incubation.

TABLE 14
Western Blot analysis

| | | Incubation Time and |
|---|---|---|
| | Reagent | Conditions |
| Blot | 100 volts for 1 hour | 1 hour at room |
| Transfer | | temperature |
| Blocking | 5% NFDM in 1x TTBS, rocking | 1 hour at room |
| | | temperature |
| Primary | Pooled serum from NHP (both IN | At least 2 hours at |
| antibody | and IM routes of Immunization) | room temperature, or |
| | diluted at 1:750 and 1:1,000 | overnight at 4° C. |
| | dilution for pooled serum from | |
| | CR in 5% NFDM - 1x TTBS, | |
| | rocking | |
| Wash | Wash 3x 5 min each in TBST | 15 min RT |
| Secondary | Goat anti-monkey IgG or Chicken | At least 1 hour at |
| antibody | Anti-Cotton rat IgG at 1:10,000 | room temperature |
| | in 5% NFDM - 1x TTBS with | |
| | rocking | |
| Wash | Wash 3x 5 min each in TBST | 15 min RT |
| Substrate | SuperSignal West Pico substrate | 5 minutes at |
| | | room temperature |

Results: Results of the Western blot analysis performed to test the ability of sera from animals immunized with RSV L19/NE01 to recognize either recombinant viral proteins, or viral antigens expressed by RSV L19 (vaccine strain), or A2 (heterologous strain) shown in FIG. 1. Recombinant F and G proteins were loaded on the gel at 200ng/well. RSV A2 and L19 were loaded on the gels based on the F protein content (200 ng/well). The blots were probed with sera from NHPs or CR as specified. Reactivity against A2-G was almost non-detectable in IM group of NHP as compared with IN group. Reactivity appears to be higher for L19-F and L19-G in IM NHP group as compared with the IN group. IM immunization with NE01/RSV L19 vaccine (either in CR, or NHP) elicited strong responses to the F and G proteins of L19 strain of the virus. The recognition of the viral proteins from A2 strain differs between sera from CR and NHP: sera from CR had a high level of antibodies against F and G with much lower levels of antibodies against other viral proteins, while sera of NHPs had a antibodies against F, N, P M, M2 and lower levels of antibodies against G.

The response to the recombinant proteins was different between two species after IM vaccination: sera from NHP had a better reaction with rF, while sera from CR had very high reactivity to rG.

Conclusion: IN immunization with NE-adjuvanted RSV L19 vaccine (NHP) induced antibodies against several viral proteins: F, G, P, N, M and M2 proteins of homologous L19 and heterologous A2 strains of RSV, indicating that the vaccine will be cross-protective against A2 if immunized via intranasal route.

IM immunization with NE01/RSV L19 vaccine (either in CR or NHP) elicited strong immune response to both strains of RSV. While the pattern of the recognition was different for CR and NHP, the ability of the sera to recognize several viral antigens, as well as two major protective viral antigens, indicate the that vaccine will offer protection against both viral strains.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

### REFERENCES

1. Hacking, D., Hull, J. 2002. Respiratory syncytial virus—virus biology and host response. J. Infection. 45: 18-24.
2. Graham, B. 2011. Biological challenges and technological opportunities for respiratory syncytial virus vaccine development. Immunological Reviews. 239: 149-166.
3. Kruijens D., Schijf, M. 2011. Local innate and adaptive immune responses regulate inflammatory cell influx into the lungs after vaccination with formalin inactivated RSV. Vaccine. 29: 2730-2741.
4. Swanson, K., Settembre, E. 2011. Structural basis for immunization with postfusion respiratory syncytial virus fusion F glycoprotein (RSV F) to elicit high neutralizing antibody titers. PNAS. 108: 9619-9624.
5. McLellan, J., Y Yang. et al., 2011. Structure of the Respiratory Syncytial Virus Fusion glycoprotein in the post-fusion conformation reveals preservation of neutralizing epitopes. J Virology. Epub. 1128/JVI00555-11.
6. Gomez, M., Mufson, M. et al. 2009. Phase-I study MEDI-534, of a live attenuated intranasal vaccine against respiratory syncytial virus and parainfluenza-3 virus in seropositive children. Ped Infect J. 28: 655-658.
7. Singh, S., Dennis, V. 2007. Immuogenicity and efficacy of recombinant RSV-F vaccine in a mouse model. Vaccine. 25: 6211-6223.
8. Kim, S., J Jang., et al., 2010. Single mucosal immunization of recombinant adenovirus-based vaccine expressing F1 protein fragment induces protective mucosal immunity against respiratory syncytial virus infection. Vaccine. 28: 3801-3808.
9. Nallet, S., Amacker, M. et al. 2009. Respiratory Syncytial Virus subunit vaccine based on a recombinant fusion protein expressed transiently in mammalian cells. 27: 6415-6419.
10. Huang K., Lawlor, H. 2010. Recombinant respiratory syncytial virus F protein expression is hindered by inefficient nuclear export and mRNA processing. Virus Genes. 40: 212-221.
11. Langley, J., Sales V., et al. 2009. A dose ranging study of a subunit Respiratory Syncytial Virus subtype A vaccine with and without aluminum phosphate adjuvantation in adults ≥65 years of age. Vaccine. 27: 5913-5919.
12. Herlocher et al. 1999 Immunological properties of plaque purified strains of live attenuated respiratory syncytial virus (RSV) for human vaccine. Vaccine, 17(2): 172-81.
13. Lukacs et al. 2006 Differential immune responses and pulmonary pathophysiology are induced by two different strains of respiratory syncytial virus. Am J Pathol 169(3): 977-86.

1. An anti- Respiratory Syncytial Virus (RSV) intravenous immunoglobulin (IVIG) composition comprising:
(a) a plurality of human antibodies comprising one or more human antibodies that bind to RSV or a fragment of RSV; and
(b) a pharmaceutically acceptable carrier;
wherein the one or more human antibodies were isolated from a donor that was immunized with a nanoemulsion RSV vaccine.

2. The IVIG composition of claim 1, wherein the plurality of human antibodies comprises at least about 90% IgG and not more than about 10% non-IgG-contaminating proteins.

3. The IVIG composition of claim 2, comprising:
(a) at least about 95% IgG, with not more than about 5% non-IgG-contaminating proteins; and/or
(b) at least about 96%, about 97%, about 98%, about 99%, or about 100% IgG.

4. (canceled)

5. The IVIG composition of claim 1, wherein:
(a) the plurality of antibodies is prepared from donor blood, serum, or plasma; and/or
(b) the plurality of antibodies comprises homologous immunoglobulins.

6. The IVIG composition of claim 1, comprising:
(a) monoclonal antibodies produced recombinantly based on the sequences of polyclonal antibodies isolated from blood, serum or plasma from a donor that was immunized with a nanoemulsion RSV vaccine; and/or
(b) polyclonal antibodies isolated from blood, serum or plasma from a donor that was immunized with a nanoemulsion RSV vaccine.

7.-8. (canceled)

9. The IVIG composition of claim 1, wherein:
(a) prior to isolation of the plurality of antibodies from blood, serum, or plasma of the donor, the donor receives more than one dose of the nanoemulsion RSV vaccine; and/or
(b) prior to isolation of the plurality of antibodies from blood, serum, or plasma of the donor, the donor receives one dose of the nanoemulsion RSV vaccine.

10. (canceled)

11. The IVIG composition of claim 1, wherein immunization of the donor with a nanoemulsion RSV vaccine results in the generation of neutralizing antibody titers, and optionally wherein the neutralizing antibody titers present in the donor range of at least 1×10² neutralization units(IU)/ml.

12. (canceled)

13. The IVIG composition of claim 1, wherein:
(a) the composition exhibits cross protection against an RSV strain not present in the nanoemulsion RSV vaccine; and/or
(b) the composition exhibits multi-RSV antigen specificity.

14. (canceled)

15. The IVIG composition of claim 1, wherein:
(a) the nanoemulsion RSV vaccine is administered via any pharmaceutically acceptable means; and/or
(b) the nanoemulsion RSV vaccine is administered to the donor parenterally or intranasally; and/or
(c) the nanoemulsion RSV vaccine is administered to the donor parenterally and the parenteral administration is by subcutaneous or intramuscular injection.

16.-17. (canceled)

18. The IVIG composition of claim 1, wherein the RSV nanoemulsion vaccine comprises:
(a) at least one RSV immunogen selected from the group consisting of whole RSV virus, an RSV antigen which is recombinant or isolated, and a combination thereof; and
(b) an immune-enhancing nanoemulsion or a dilution thereof, comprising droplets having an average diameter of 1000 nm or less, wherein the at least one RSV immunogen is comprised within the nanoemulsion, wherein the nanoemulsion comprises:
(i) an aqueous phase;
(ii) at least one oil;
(iii) at least one surfactant; and
(iv) at least one organic solvent.

19. The IVIG composition of claim 18, wherein:
(a) the nanoemulsion inactivates the RSV immunogen; and/or
(b) the RSV immunogen is from an RSV strain which is resistant to one or more antiviral drugs; and/or
(c) the nanoemulsion RSV vaccine comprises an RSV immunogen from an RSV strain selected from the group consisting of (1) Human RSV strain A2, deposited under ATCC No. VR-1540; (2) Human RSV strain Long, deposited under ATCC No. VR-26; (3) Bovine RSV strain A 51908, deposited under ATCC No. VR-794; (4) Human RSV strain 9320, deposited under ATCC No. VR-955; (5) Bovine RSV strain 375, deposited under ATCC No. VR-1339; (6) Human RSV strain B WV/14617/85, deposited under ATCC No. VR-1400; (7) Bovine RSV strain Iowa (FS1-1), deposited under ATCC No. VR-1485; (8) Caprine RSV strain GRSV, deposited under ATCC No. VR-1486; (9) Human RSV strain 18537, deposited under ATCC No. VR-1580; (10) Human RSV strain A2, deposited under ATCC No. VR-1540P; (11) Human RSV mutant strain A2 cpts-248, deposited under ATCC No. VR-2450; (12) Human RSV mutant strain A2 cpts-530/1009, deposited under ATCC No. VR-2451; (13) Human RSV mutant strain A2 cpts-530, deposited under ATCC No. VR-2452; (14) Human RSV mutant strain A2 cpts-248/955, deposited under ATCC No. VR-2453; (15) Human RSV mutant strain A2 cpts-248/404, deposited under ATCC No. VR-2454; (16) Human RSV mutant strain A2 cpts-530/1030, deposited under ATCC No. VR-2455; (17) RSV mutant strain subgroup B cp23 Clone 1A2, deposited under ATCC No. VR-2579; and (18) Human RSV mutant strain Subgroup B, Strain B1, cp52 Clone 2B5, deposited under ATCC No. VR-2542; and/or
(d) the RSV immunogen is an RSV subunit selected from the group consisting of F protein, G protein, SH protein, nucleoprotein, phosphoprotein, matrix protein, large protein, and an immunogenic fragments of any of these proteins.

20.-22. (canceled)

23. The IVIG composition of claim 18, wherein:
(a) the RSV immunogen comprises F protein, G protein, or an immunogenic fragment of F or G protein; and/or
(b) the at least one RSV immunogen is derived from RSV strain L19 (RSV-L19); and/or
(c) the RSV vaccine comprises whole RSV virus, which optionally is RSV-L19.

24.-26. (canceled)

27. The IVIG composition of claim 23, wherein the RSV-L19 virus:
(a) is a hyperproducer of Fusion (F) and Glycoprotein (G) structural proteins associated with viral particles; and/or
(b) is attenuated human respiratory syncytial virus (RSV) strain L19; and/or
(c) is deposited with the American Type Culture Collection (ATCC) as RSV-L19.

28.-29. (canceled)

30. The IVIG composition of claim 18, wherein:
(a) the nanoemulsion RSV vaccine comprises an RSV immunogen from more than one strain of RSV; and/or
(b) the nanoemulsion RSV vaccine comprises as an RSV immunogen RSV whole virus and at least one RSV viral subunit; and/or
(c) the nanoemulsion RSV vaccine comprises a RSV immunogen from an RSV virus comprising at least one attenuating mutation.

31.-32. (canceled)

33. The IVIG composition of claim 18, wherein the nanoemulsion:
(a) is not systemically toxic to the subject;
(b) produces minimal or no inflammation upon administration; or
(c) a combination thereof.

34. The IVIG composition of claim 18, wherein the nanoemulsion or a dilution thereof comprises:
(a) an aqueous phase;
(b) about 1% to about 80% oil;
(c) about 0.1% organic solvent to about 50% organic solvent; and
(d) about 0.001% surfactant to about 10% surfactant.

35. The IVIG composition of claim 18, wherein: the nanoemulsion further comprises a cationic surfactant.

36. The IVIG composition of claim 18, wherein the organic solvent:
(a) is selected from the group consisting of a C₁-C₁₂ alcohol, diol, triol, dialkyl phosphate, tri-alkyl phosphate, and combinations thereof;
(b) is an alcohol selected from the group consisting of a nonpolar solvent, a polar solvent, a protic solvent, an aprotic solvent, semi-synthetic derivatives thereof, and combinations thereof;
(c) is selected from the group consisting of tri-n-butyl phosphate, ethanol, methanol, isopropyl alcohol, glycerol, medium chain triglycerides, diethyl ether, ethyl acetate, acetone, dimethyl sulfoxide (DMSO), acetic acid, n-butanol, butylene glycol, perfumers alcohols, isopropanol, n-propanol, formic acid, propylene glycols, glycerol, sorbitol, industrial methylated spirit, triacetin, hexane, benzene, toluene, diethyl ether, chloroform, 1,4-dixoane, tetrahydrofuran, dichloromethane, acetone, acetonitrile, dimethylformamide, dimethyl sulfoxide, formic acid, semi-synthetic derivatives thereof, and any combination thereof; or
(d) any combination thereof.

37. The IVIG composition of claim 18, wherein the oil is:
(a) any cosmetically or pharmaceutically acceptable oil;
(b) non-volatile;
(c) selected from the group consisting of animal oil, vegetable oil, natural oil, synthetic oil, hydrocarbon oils, silicone oils, and semi-synthetic derivatives thereof;
(d) selected from the group consisting of mineral oil, squalene oil, flavor oils, silicon oil, essential oils, water insoluble vitamins, Isopropyl stearate, Butyl stearate, Octyl palmitate, Cetyl palmitate, Tridecyl behenate, Diisopropyl adipate, Dioctyl sebacate, Menthyl anthranhilate, Cetyl octanoate, Octyl salicylate, Isopropyl myristate, neopentyl glycol dicarpate cetols, Ceraphyls®, Decyl oleate, diisopropyl adipate, C₁₂₋₁₅ alkyl lactates, Cetyl lactate, Lauryl lactate, Isostearyl neopentanoate, Myristyl lactate, Isocetyl stearoyl stearate, Octyldodecyl stearoyl stearate, Hydrocarbon oils, Isoparaffin, Fluid paraffins, Isododecane, Petrolatum, Argan oil, Canola oil, Chile oil, Coconut oil, corn oil, Cottonseed oil, Flaxseed oil, Grape seed oil, Mustard oil, Olive oil, Palm oil, Palm kernel oil, Peanut oil, Pine seed oil, Poppy seed oil, Pumpkin seed oil, Rice bran oil, Safflower oil, Tea oil, Truffle oil, Vegetable oil, Apricot (kernel) oil, Jojoba oil (simmondsia chinensis seed oil), Grapeseed oil, Macadamia oil, Wheat germ oil, Almond oil, Rapeseed oil, Gourd oil, Soybean oil, Sesame oil, Hazelnut oil, Maize oil, Sunflower oil, Hemp oil, Bois oil, Kuki nut oil, Avocado oil, Walnut oil, Fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, wintergreen leaf oil, flower oil, chamomile oil, clary sage oil, clove oil, geranium flower oil, hyssop flower oil, jasmine flower oil, lavender flower oil, manuka flower oil, Marhoram flower oil, orange flower oil, rose flower oil, ylang-ylang flower oil, Bark oil, cassia Bark oil, cinnamon bark oil, sassafras Bark oil, Wood oil, camphor wood oil, cedar wood oil, rosewood oil, sandalwood oil), rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, Oleic acid, Linoleic acid, Oleyl alcohol, Isostearyl alcohol, semi-synthetic derivatives thereof, and combinations thereof; or
(e) any combination thereof.

38. The IVIG composition of claim 18, wherein:
(a) the surfactant is selected from the group consisting of ethoxylated nonylphenol comprising 9 to 10 units of ethyleneglycol, ethoxylated undecanol comprising 8 units of ethyleneglycol, polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan monolaurate, sorbitan monopalmitate, sorbitan monostearate, sorbitan monooleate, ethoxylated hydrogenated ricin oils, sodium laurylsulfate, a diblock copolymer of ethyleneoxyde and propyleneoxyde, Ethylene Oxide-Propylene Oxide Block Copolymers, and tetra-functional block copolymers based on ethylene oxide and propylene oxide, Glyceryl monoesters, Glyceryl caprate, Glyceryl caprylate, Glyceryl cocate, Glyceryl erucate, Glyceryl hydroxysterate, Glyceryl isostearate, Glyceryl lanolate, Glyceryl laurate, Glyceryl linolate, Glyceryl myristate, Glyceryl oleate, Glyceryl PABA, Glyceryl palmitate, Glyceryl ricinoleate, Glyceryl stearate, Glyceryl thiglycolate, Glyceryl dilaurate, Glyceryl dioleate, Glyceryl dimyristate, Glyceryl disterate, Glyceryl sesuioleate, Glyceryl stearate lactate, Polyoxyethylene cetyl/stearyl ether, Polyoxyethylene cholesterol ether, Polyoxyethylene laurate or dilaurate, Polyoxyethylene stearate or distearate, polyoxyethylene fatty ethers, Polyoxyethylene lauryl ether, Polyoxyethylene stearyl ether, polyoxyethylene myristyl ether, a steroid, Cholesterol, Betasitosterol, Bisabolol, fatty acid esters of alcohols, isopropyl myristate, Aliphati-isopropyl n-butyrate, Isopropyl n-hexanoate, Isopropyl n-decanoate, Isoproppyl palmitate, Octyldodecyl myristate, alkoxylated alcohols, alkoxylated acids, alkoxylated amides, alkoxylated sugar derivatives, alkoxylated derivatives of natural oils and waxes, polyoxyethylene polyoxypropylene block copolymers, nonoxynol-14, PEG-8 laurate, PEG-6 Cocoamide, PEG-20 methylglucose sesquistearate, PEG40 lanolin, PEG-40 castor oil, PEG-40 hydrogenated castor oil, polyoxyethylene fatty ethers, glyceryl diesters, polyoxyethylene stearyl ether, polyoxyethylene myristyl ether, and polyoxyethylene lauryl ether, glyceryl dilaurate, glyceryl dimystate, glyceryl distearate, semi-synthetic derivatives thereof, and mixtures thereof;
(b) the surfactant is a non-ionic lipid selected from the group consisting of glyceryl laurate, glyceryl myristate, glyceryl dilaurate, glyceryl dimyristate, semi-synthetic derivatives thereof, and mixtures thereof;
(c) the surfactant is a polyoxyethylene fatty ether having a polyoxyethylene head group ranging from about 2 to about 100 groups;
(d) the surfactant is an alkoxylated alcohol having the structure shown in formula I below:
R5—(OCH2 CH2)_{y}—OH [Formula I]
wherein R₅ is a branched or unbranched alkyl group having from about 6 to about 22 carbon atoms and y is between about 4 and about 100, and preferably, between about 10 and about 100;
(e) the surfactant is an alkoxylated alcohol which is an ethoxylated derivative of lanolin alcohol;
(f) the surfactant is nonionic and is selected from the group consisting of nonoxynol-9, an ethoxylated surfactant, an alcohol ethoxylated, an alkyl phenol ethoxylated, a fatty acid ethoxylated, a monoalkaolamide ethoxylated, a sorbitan ester ethoxylated, a fatty amino ethoxylated, an ethylene oxide-propylene oxide copolymer, Bis(polyethylene glycol bis[imidazoyl carbonyl]), Brij® 35, Brij® 56, Brij® 72, Brij® 76, Brij® 92V, Brij® 97, Brij® 58P, Cremophor® EL, Decaethylene glycol monododecyl ether, N-Decanoyl-N-methylglucamine, n-Decyl alpha-D-glucopyranoside, Decyl beta-D-maltopyranoside, n-Dodecanoyl-N-methylglucamide, n-Dodecyl alpha-D-maltoside, n-Dodecyl beta-D-maltoside, Heptaethylene glycol monodecyl ether, Heptaethylene glycol monotetradecyl ether, Heptaethylene glycol monododecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Methyl-6-O-(N-heptylcarbamoyl)-alpha-D-glucopyranoside, Nonaethylene glycol monododecyl ether, N-Nonanoyl-N-methylglucamine, Octaethylene glycol monodecyl ether, Octaethylene glycol monododecyl ether, Octaethylene glycol monohexadecyl ether, Octaethylene glycol monooctadecyl ether, Octaethylene glycol monotetradecyl ether, Octyl-beta-D-glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin from Quillaja bark, Span® 20, Span® 40, Span® 60, Span® 65, Span® 80, Span® 85, Tergitol, Tergitol Type 15-S-12, Tergitol Type 15-S-30, Tergitol Type 15-S-5, Tergitol Type 15-S-7, Tergitol Type 15-S-9, Tergitol Type NP-10, Tergitol Type NP-4, Tergitol Type NP-40, Tergitol Type NP-7, Tergitol Type NP-9, Tergitol Type TMN-10, Tergitol Type TMN-6, Tetradecyl-beta-D-maltoside, Tetraethylene glycol monodecyl ether, Tetraethylene glycol monododecyl ether, Tetraethylene glycol monotetradecyl ether, Triethylene glycol monodecyl ether, Triethylene glycol monododecyl ether, Triethylene glycol monohexadecyl ether, Triethylene glycol monooctyl ether, Triethylene glycol monotetradecyl ether, Triton CF-21, Triton CF-32, Triton DF-12, Triton DF-16, Triton GR-5M, Triton QS-15, Triton QS-44, Triton X-100, Triton X-102, Triton X-15, Triton X-151, Triton X-200, Triton X-207, Triton X-114, Triton X-165, Triton X-305, Triton X-405, Triton X-45, Triton X-705-70, TWEEN® 20, TWEEN® 21, TWEEN® 40, TWEEN® 60, TWEEN® 61, TWEEN® 65, TWEEN® 80, TWEEN® 81, TWEEN® 85, Tyloxapol, n-Undecyl beta-D-glucopyranoside, Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, Poloxamer 407, Poloxamer 105 Benzoate, Poloxamer 182, Dibenzoate, semi-synthetic derivatives thereof, and combinations thereof;
(g) the surfactant is cationic and is selected from the group consisting of a quarternary ammonium compound, an alkyl trimethyl ammonium chloride compound, a dialkyl dimethyl ammonium chloride compound, Benzalkonium chloride, Benzyldimethylhexadecylammonium chloride, Benzyldimethyltetradecylammonium chloride, Benzyldodecyldimethylammonium bromide, Benzyltrimethylammonium tetrachloroiodate, Cetylpyridinium chloride, Dimethyldioctadecylammonium bromide, Dodecylethyldimethylammonium bromide, Dodecyltrimethylammonium bromide, Ethylhexadecyldimethylammonium bromide, Girard's reagent T, Hexadecyltrimethylammonium bromide, N,N′,N′-Polyoxyethylene(10)-N-tallow-1,3-diaminopropane, Thonzonium bromide, Trimethyl(tetradecyl)ammonium bromide, 1,3,5-Triazine-1,3,5(2H,4H,6H)-triethanol, 1-Decanaminium, N-decyl-N, N-dimethyl-, chloride, Didecyl dimethyl ammonium chloride, 2-(2-(p-(Diisobutyl)creso sxy)ethoxy)ethyl dimethyl benzyl ammonium chloride, 2-(2-(p-(Diisobutyl)phenoxy)ethoxy)ethyl dimethyl benzyl ammonium chloride, Alkyl 1 or 3 benzyl-1-(2-hydroxethyl)-2-imidazolinium chloride, Alkyl bis(2-hydroxyethyl) benzyl ammonium chloride, Alkyl demethyl benzyl ammonium chloride, Alkyl dimethyl 3,4-dichlorobenzyl ammonium chloride (100% Cu), Alkyl dimethyl 3,4-dichlorobenzyl ammonium chloride (50% C₁₄, 40% C₁₂, 10% C₁₆), Alkyl dimethyl 3,4-dichlorobenzyl ammonium chloride (55% C₁₄, 23% C₁₂, 20% C₁₆), Alkyl dimethyl benzyl ammonium chloride, Alkyl dimethyl benzyl ammonium chloride (100% C₁₄), Alkyl dimethyl benzyl ammonium chloride (100% C₁₆), Alkyl dimethyl benzyl ammonium chloride (41% C₁₄, 28% Cu), Alkyl dimethyl benzyl ammonium chloride (47% C₁₂, 18% C14), Alkyl dimethyl benzyl ammonium chloride (55% C₁₆, 20% C₁₄), Alkyl dimethyl benzyl ammonium chloride (58% C₁₄, 28% C₁6), Alkyl dimethyl benzyl ammonium chloride (60% C₁₄, 25% C12), Alkyl dimethyl benzyl ammonium chloride (61% C₁₁, 23% C₁₄), Alkyl dimethyl benzyl ammonium chloride (61% Cu, 23% C₁₄), Alkyl dimethyl benzyl ammonium chloride (65% C₁₂, 25% C₁₄), Alkyl dimethyl benzyl ammonium chloride (67% C₁₂, 24% C₁₄), Alkyl dimethyl benzyl ammonium chloride (67% C₁₂, 25% C₁₄), Alkyl dimethyl benzyl ammonium chloride (90% C14, 5% Cu), Alkyl dimethyl benzyl ammonium chloride (93% C₁₄, 4% C₁₂), Alkyl dimethyl benzyl ammonium chloride (95% C₁₆, 5% C₁₈), Alkyl didecyl dimethyl ammonium chloride, Alkyl dimethyl benzyl ammonium chloride (C₁₂₋₁₆), Alkyl dimethyl benzyl ammonium chloride (C₁₂₋₁₈), dialkyl dimethyl benzyl ammonium chloride, Alkyl dimethyl dimethybenzyl ammonium chloride, Alkyl dimethyl ethyl ammonium bromide (90% C₁₄, 5% C₁₆, 5% C₁₂), Alkyl dimethyl ethyl ammonium bromide (mixed alkyl and alkenyl groups as in the fatty acids of soybean oil), Alkyl dimethyl ethylbenzyl ammonium chloride, Alkyl dimethyl ethylbenzyl ammonium chloride (60% C₁₄), Alkyl dimethyl isopropylbenzyl ammonium chloride (50% C₁₂, 30% C₁₄, 17% C₁₆, 3% C18), Alkyl trimethyl ammonium chloride (58% C₁₈, 40% C₁₆, 1% C₁₄, 1% Cu), Alkyl trimethyl ammonium chloride (90% C₁₈, 10% C₁₆), Alkyldimethyl(ethylbenzyl) ammonium chloride (C₁₂₋₁₈), Di-(C₈₋₁₀)-alkyl dimethyl ammonium chlorides, Dialkyl dimethyl ammonium chloride, Dialkyl methyl benzyl ammonium chloride, Didecyl dimethyl ammonium chloride, Diisodecyl dimethyl ammonium chloride, Dioctyl dimethyl ammonium chloride, Dodecyl bis (2-hydroxyethyl) octyl hydrogen ammonium chloride, Dodecyl dimethyl benzyl ammonium chloride, Dodecylcarbamoyl methyl dinethyl benzyl ammonium chloride, Heptadecyl hydroxyethylimidazolinium chloride, Hexahydro-1,3,5-tris(2-hydroxyethyl)-s-triazine, Myristalkonium chloride (and) Quat RNIUM 14, N,N-Dimethyl-2-hydroxypropylammonium chloride polymer, n-Tetradecyl dimethyl benzyl ammonium chloride monohydrate, Octyl decyl dimethyl ammonium chloride, Octyl dodecyl dimethyl ammonium chloride, Octyphenoxyethoxyethyl dimethyl benzyl ammonium chloride, Oxydiethylenebis(alkyl dimethyl ammonium chloride), Trimethoxysily propyl dimethyl octadecyl ammonium chloride, Trimethoxysilyl quats, Trimethyl dodecylbenzyl ammonium chloride, semi-synthetic derivatives thereof, and combinations thereof;
(h) the surfactant is anionic and is selected from the group consisting of a carboxylate, a sulphate, a sulphonate, a phosphate, Chenodeoxycholic acid, Chenodeoxycholic acid sodium salt, Cholic acid, ox or sheep bile, Dehydrocholic acid, Deoxycholic acid, Deoxycholic acid methyl ester, Digitonin, Digitoxigenin, N,N-Dimethyldodecylamine N-oxide, Docusate sodium salt, Glycochenodeoxycholic acid sodium salt, Glycocholic acid hydrate, synthetic, Glycocholic acid sodium salt hydrate, synthetic, Glycodeoxycholic acid monohydrate, Glycodeoxycholic acid sodium salt, Glycolithocholic acid 3-sulfate disodium salt, Glycolithocholic acid ethyl ester, N-Lauroylsarcosine sodium salt, N-Lauroylsarcosine solution, Lithium dodecyl sulfate, Lugol solution, Niaproof 4, Type 4, 1-Octanesulfonic acid sodium salt, Sodium 1-butanesulfonate, Sodium 1-decanesulfonate, Sodium 1-dodecanesulfonate, Sodium 1-heptanesulfonate anhydrous, Sodium 1-nonanesulfonate, Sodium 1-propanesulfonate monohydrate, Sodium 2-bromoethanesulfonate, Sodium cholate hydrate, Sodium choleate, Sodium deoxycholate, Sodium deoxycholate monohydrate, Sodium dodecyl sulfate, Sodium hexanesulfonate anhydrous, Sodium octyl sulfate, Sodium pentanesulfonate anhydrous, Sodium taurocholate, Taurochenodeoxycholic acid sodium salt, Taurodeoxycholic acid sodium salt monohydrate, Taurohyodeoxycholic acid sodium salt hydrate, Taurolithocholic acid 3-sulfate disodium salt, Tauroursodeoxycholic acid sodium salt, Trizma® dodecyl sulfate, Ursodeoxycholic acid, semi-synthetic derivatives thereof, and combinations thereof;
(i) the surfactant is zwitterionic and is selected from the group consisting of an N-alkyl betaine, lauryl amindo propyl dimethyl betaine, an alkyl dimethyl glycinate, an N-alkyl amino propionate, CHAPS (minimum 98%), CHAPSO (minimum 98%), 3-(Decyldimethylammonio)propanesulfonate inner salt, 3-(Dodecyldimethylammonio)propanesulfonate inner salt, 3-(N,N-Dimethylmyristylammonio)propanesulfonate, 3-(N,N-Dimethyloctadecylammonio)propanesulfonate, 3-(N,N-Dimethyloctylammonio)propanesulfonate inner salt, 3-(N,N-Dimethylpalmityammonio)propanesulfonate, semi-synthetic derivatives thereof, and combinations thereof;
(j) the surfactant is polymeric and the polymeric surfactant is selected from the group consisting of a graft copolymer of a poly(methyl methacrylate) backbone with at least one polyethylene oxide (PEO) side chain, polyhydroxystearic acid, an alkoxylated alkyl phenol formaldehyde condensate, a polyalkylene glycol modified polyester with fatty acid hydrophobes, a polyester, semi-synthetic derivatives thereof, and combinations thereof; or
(k) any combination thereof.

39. The IVIG composition of claim 18, wherein the nanoemulsion:
(a) comprises at least one cationic surfactant;
(b) comprises a cationic surfactant which is cetylpyridinium chloride;
(c) comprises a cationic surfactant, and wherein the concentration of the cationic surfactant is less than about 5.0% and greater than about 0.001%;
(d) comprises a cationic surfactant, and wherein the concentration of the cationic surfactant is selected from the group consisting of less than about 5%, less than about 4.5%, less than about 4.0%, less than about 3.5%, less than about 3.0%, less than about 2.5%, less than about 2.0%, less than about 1.5%, less than about 1.0%, less than about 0.90%, less than about 0.80%, less than about 0.70%, less than about 0.60%, less than about 0.50%, less than about 0.40%, less than about 0.30%, less than about 0.20%, less than about 0.10%, greater than about 0.001%.,greater than about 0.002%, greater than about 0.003%, greater than about 0.004%, greater than about 0.005%, greater than about 0.006%, greater than about 0.007%, greater than about 0.008%, greater than about 0.009%, and greater than about 0.010%; or
(e) any combination thereof.

40. The IVIG composition of claim 18, wherein:
(a) the nanoemulsion comprises at least one cationic surfactant and at least one non-cationic surfactant;
(b) the nanoemulsion comprises at least one cationic surfactant and at least one non-cationic surfactant, wherein the non-cationic surfactant is a nonionic surfactant;
(c) the nanoemulsion comprises at least one cationic surfactant and at least one non-cationic surfactant, wherein the non-cationic surfactant is a polysorbate nonionic surfactant, a poloxamer nonionic surfactant, or a combination thereof;
(d) the nanoemulsion comprises at least one cationic surfactant and at least one nonionic surfactant which is polysorbate 20, polysorbate 80, poloxamer 188, poloxamer 407, or a combination thereof;
(e) the nanoemulsion comprises at least one cationic surfactant and at least one nonionic surfactant which is polysorbate 20, polysorbate 80, poloxamer 188, poloxamer 407, or a combination thereof, and wherein the nonionic surfactant is present at about 0.01% to about 5.0% , or at about 0.1% to about 3%;
(f) the nanoemulsion comprises at least one cationic surfactant and at least one non-cationic surfactant, wherein the non-cationic surfactant is a nonionic surfactant, and the non-ionic surfactant is present in a concentration of about 0.05% to about 10%, about 0.05% to about 7.0%, about 0.1% to about 7%, or about 0.5% to about 4%;
(g) the nanoemulsion comprises at least one cationic surfactant and at least one a nonionic surfactant, wherein the cationic surfactant is present in a concentration of about 0.05% to about 2% or about 0.01% to about 2%; or
(h) any combination thereof.

41. A method of treating or preventing respiratory syncytial virus (RSV) in a subject comprising administering to a subject an anti-RSV IVIG composition according to claim 1.

42. The method of claim 41, wherein:
(a) the anti-RSV IVIG is administered intravenously; and/or
(b) the subject is an infant, child, elderly, or immunocompromised; and/or
(c) the subject is already infected with RSV; and/or
(d) the subject is already infected with RSV; and/or
(e) the subject is at risk of being infected with RSV

43.-45. (canceled)

46. A method of preparing a pharmaceutical composition comprising intravenous immunoglobulin (IVIG) for treatment of respiratory syncytial virus (RSV), the method comprising:
(a) administering to a donor with a RSV vaccine;
(b) isolating from the donor a plurality of antibodies that bind to RSV or a fragment of RSV;
(c) formulating the plurality of antibodies into an IVIG composition.

47. The method of claim 46, wherein:
(a) the RSV vaccine is a nanoemulsion vaccine; and/or
(b) the RSV vaccine is administered to the donor in a dose corresponding to 1 μg or more of F protein; and/or
(c) the RSV vaccine is administered to the donor in a dose corresponding to 20 μg or more of F protein; and/or
(d) the RSV vaccine is administered to the donor in a dose corresponding to 50 μg or more of F protein; and/or
(e) the RSV vaccine is administered to the donor in a dose corresponding to 100 μg or more of F protein; and/or
(f) the RSV vaccine is administered to the donor subcutaneously, intramuscularly, or nasally.

48.-54. (canceled)

55. The method of claim 46, wherein the nanoemulsion RSV vaccine comprises an RSV immunogen from an RSV strain selected from the group consisting of (1) Human RSV strain A2, deposited under ATCC No. VR-1540; (2) Human RSV strain Long, deposited under ATCC No. VR-26; (3) Bovine RSV strain A 51908, deposited under ATCC No. VR-794; (4) Human RSV strain 9320, deposited under ATCC No. VR-955; (5) Bovine RSV strain 375, deposited under ATCC No. VR-1339; (6) Human RSV strain B WV/14617/85, deposited under ATCC No. VR-1400; (7) Bovine RSV strain Iowa (FS1-1), deposited under ATCC No. VR-1485; (8) Caprine RSV strain GRSV, deposited under ATCC No. VR-1486; (9) Human RSV strain 18537, deposited under ATCC No. VR-1580; (10) Human RSV strain A2, deposited under ATCC No. VR-1540P; (11) Human RSV mutant strain A2 cpts-248, deposited under ATCC No. VR-2450; (12) Human RSV mutant strain A2 cpts-530/1009, deposited under ATCC No. VR-2451; (13) Human RSV mutant strain A2 cpts-530, deposited under ATCC No. VR-2452; (14) Human RSV mutant strain A2 cpts-248/955, deposited under ATCC No. VR-2453; (15) Human RSV mutant strain A2 cpts-248/404, deposited under ATCC No. VR-2454; (16) Human RSV mutant strain A2 cpts-530/1030, deposited under ATCC No. VR-2455; (17) RSV mutant strain subgroup B cp23 Clone 1A2, deposited under ATCC No. VR-2579; and (18) Human RSV mutant strain Subgroup B, Strain B1,cp52 Clone 2B5, deposited under ATCC No. VR-2542.

56. The method of claim 46, wherein:
(a) the RSV vaccine comprises an RSV subunit selected from the group consisting of F protein, G protein, SH protein, nucleoprotein, phosphoprotein, matrix protein, large protein, and an immunogenic fragments of any of these proteins; and/or
(b) the RSV vaccine comprises F protein, G protein, or an immunogenic fragment of F or G protein; and/or
(c) the RSV vaccine comprises at least one RSV immunogen derived from RSV-L19; and/or
(d) the RSV vaccine comprises whole RSV virus, which optionally is RSV-L19.

57.-60. (canceled)
